(19) United States
(12) Patent Application Publication
(10) Pub. No.: US 2023/0302058 A1
(43) Pub. Date: Sep. 28, 2023

(54) TREATMENT OF LYMPHEDEMA AND RELATED CONDITIONS USING PLACENTAL ADHERENT CELLS

(71) Applicant: Celularity Inc., Florham Park, NJ (US)

(72) Inventors: Robert J. HARIRI, Bernardsville, NJ (US); Shuyang HE, Martinsville, NJ (US)

(73) Assignee: Celularity Inc., Florham Park, NJ (US)

(21) Appl. No.: 18/201,907

(22) Filed: May 25, 2023

Related U.S. Application Data
(63) Continuation of application No. 16/467,026, filed on Jun. 5, 2019, now abandoned.
(60) Provisional application No. 62/430,123, filed on Dec. 5, 2016.

(51) Int. Cl. A61K 35/50; A61P 7/10; C12N 5/073

(52) U.S. Cl. CPC A61K 35/50; A61P 7/10; C12N 5/0605; C12N 2500/02; C12N 2502/025; C12N 2502/28

(57) ABSTRACT

Provided herein are methods of using tissue culture plastic-adherent placental adherent cells, e.g. placental stem cells, referred to herein as placental adherent cells, to treat lymphedema and lymphedema-related disorders.

### 1. FIELD

Provided herein are methods of using tissue culture plastic-adherent placental adherent cells, e.g. placental stem cells, referred to herein as placental adherent cells, to treat lymphedema and lymphedema-related disorders.

### 2. BACKGROUND

The placenta is a particularly attractive source of stem cells. Because mammalian placentas are plentiful and are normally discarded as medical waste, they represent a unique source of medically-useful stem cells. Provided herein are such isolated placental stem cells, populations of the placental stem cells, and methods of using the same to treat lymphedema and diseases or disorders treatable by treating lymphedema.

### 3. SUMMARY

Provided herein are methods inducing lymphangiogenesis in subjects, e.g., human subjects, comprising administration of tissue culture plastic-adherent placental adherent cells, e.g., placental adherent cells (the placental adherent cells described in Section 5.3, below).

In a specific embodiment, provided herein is a method of treating an individual having lymphedema, or a disease or disorder treatable by treating lymphedema, comprising administering to the individual a therapeutically effective amount of tissue culture plastic-adherent placental adherent cells, e.g., placental adherent cells (the placental adherent cells described in Section 5.3, below) in an amount and for a time sufficient for detectable improvement of the lymphedema, or one or more symptoms of said disease or disorder. In certain embodiments, the method comprises administering placental adherent cells to said individual in an amount and for a time sufficient for an improvement of one or more of lymphedema-related or lymphedema-caused symptoms or sequelae such as swelling of part or all of one or both legs, swelling of part or all of one or both arms, heaviness or tightness in an arm or leg, nontender pitted edema of a leg or arm, restricted motion or a sensation of restricted motion in an arm or leg, aching or discomfort in an arm or leg, infection in or below the skin, cellulitis, lymphangitis, lymphorrhea, impetigo, erythema of the skin of an affected arm or leg, a positive stemmer's sign, or hardening and/or thickening of the skin of an affected arm or leg, as compared to the individual prior to administration of said placental adherent cells. In certain embodiments, the lymphedema is Stage 0, Stage 1, Stage 2, or Stage 3 lymphedema. In certain other embodiments, the lymphedema is adjunct to lipedema.

In certain embodiments, the edema treated by administration of placental adherent cells is not edema caused by, or associated with, venous insufficiency, heart failure, sleep apnea, or kidney failure.

In certain embodiments, ability of placental adherent cells to induce lymphangiogenesis in a subject, and/or treat lymphedema, or a disease or disorder treatable by treating lymphedema, in a subject, is confirmed by measurement of LYVE-1 (lymphatic vessel endothelial hyaluronan receptor-1) expression levels in the subject.

In another specific embodiment of the methods of inducing lymphangiogenesis and methods of treatment described herein, placental adherent cells are administered to an individual by injection, e.g., injection into a limb affected by lymphedema. In another specific embodiment, placental adherent cells are administered intramuscularly.

In another specific embodiment of the methods of inducing lymphangiogenesis and methods of treatment described herein, placental adherent cells are administered to an individual by intravenous infusion. In specific embodiment, said intravenous infusion is intravenous infusion over about 1 to about 8 hours.

In other specific embodiments of the methods of inducing lymphangiogenesis and methods of treatment described herein, placental adherent cells are administered subcutaneously, intradermally, intra-arterially, intrathecally, or intraperitoneally; or by implantation in said individual of a matrix or scaffold comprising placental adherent cells.

In certain embodiments of the methods of inducing lymphangiogenesis and methods of treatment described herein, about 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, or 1×10¹⁰ placental adherent cells are administered to an individual. In certain embodiments, 1×10⁴ to 1×10⁵, 1×10⁵ to 1×10⁶, 1×10⁶ to 1×10⁷, 1×10⁷ to 1×10⁸, 1×10⁸ to 1×10⁹, or 1×10⁹ to 1×10¹⁰ placental adherent cells are administered to an individual.

In another specific embodiment of the methods of inducing lymphangiogenesis and methods of treatment described herein, said placental adherent cells have been proliferated in vitro for no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 population doublings. In another specific embodiment, said placental adherent cells are from a culture of said cells that has been passaged at least or about 1, 2, 3, 4, 5, 6, 7, 8, 9, 10, 11, 12, 13, 14, 15, 16, 17, 18, 19, or 20 times, or more.

In another embodiment of the methods of inducing lymphangiogenesis and methods of treatment described herein, said placental adherent cells have been cryopreserved and thawed prior to being administered to the subject.

In a specific embodiment, the placental adherent cells used in accordance with the methods of inducing lymphangiogenesis and methods of treatment described herein adhere to tissue culture plastic and are CD34⁻, CD10⁺, CD105⁺ and CD200⁺, as detectable by flow cytometry. In certain embodiments, the placental adherent cells have the capacity to differentiate into osteogenic or chondrogenic cells. In another embodiment, said placental adherent cells are adherent to tissue culture plastic; CD34⁻, CD10⁺, CD105⁺ and CD200⁺ as detectable by flow cytometry; and have the capacity to differentiate into cells having one or more characteristics of osteogenic or chondrogenic cells, e.g., characteristics of osteocytes or chondrocytes. In other embodiments, the placental adherent cells additionally have the ability to differentiate into cells having one or more characteristics of neural cells or neurogenic cells, e.g., characteristics of neurons; one or more characteristics of glial cells, e.g., characteristics of glia or astrocytes; one or more characteristics of adipocytic cells, e.g., characteristics of adipocytes; one or more characteristics of pancreatic cells; and/or one or more characteristics of cardiac cells.

In certain embodiments, the placental adherent cells used in accordance with the methods of inducing lymphangiogenesis and methods of treatment described herein are CD34⁻, CD10⁺, CD105⁺ and CD200⁺, and one or more of CD38⁻, CD45⁻, CD80⁻, CD86⁻, CD133⁻, HLA-DR,DP,DQ⁻, SSEA3⁻, SSEA4⁻, CD29⁺, CD44⁺, CD73⁺, CD90⁺, CD105⁺, HLA-A,B,C⁺, PDL1⁺, ABC-p⁺, and/or OCT-4⁺, as detectable by flow cytometry and/or RT-PCR. In another embodiment, said placental adherent cells are CD34⁻, CD45⁻, CD10⁺, CD90⁺, CD105⁺ and CD200⁺, as detectable by flow cytometry. In another embodiment, said placental adherent cells are CD34⁻, CD45⁻, CD10⁺, CD80⁻, CD86⁻, CD90⁺, CD105⁺ and CD200⁺, as detectable by flow cytometry. In another embodiment, said placental adherent cells are CD34⁻, CD45⁻, CD10⁺, CD80⁻, CD86⁻, CD90⁺, CD105⁺ and CD200⁺, and additionally one or more of CD29⁺, CD38⁻, CD44⁺, CD54⁺, SH3⁺ or SH4⁺, as detectable by flow cytometry. In another embodiment, said placental adherent cells are CD34⁻, CD38⁻, CD45⁻, CD10⁺, CD29⁺, CD44⁺, CD54⁺, CD73⁺, CD80⁻, CD86⁻, CD90⁺, CD105⁺, and CD200⁺ as detectable by flow cytometry.

In certain embodiments, the placental adherent cells used in accordance with the methods of inducing lymphangiogenesis and methods of treatment described herein are CD34⁻, CD10⁺, CD105⁺ and CD200⁺ placental adherent cells and are additionally one or more of CD117⁻, CD133⁻, KDR⁻ (VEGFR2⁻), HLA-A,B,C⁺, HLA-DP,DQ,DR⁻, or Programmed Death-1 Ligand (PDL1)⁺, or any combination thereof. In another specific embodiment, said placental adherent cells are CD34⁻, CD38⁻, CD45⁻, CD10⁺, CD29⁺, CD44⁺, CD54⁺, CD73⁺, CD80⁻, CD86⁻, CD90⁺, CD105⁺, CD117⁻, CD133⁻, CD200⁺, KDR⁻ (VEGFR2⁻), HLA-A,B,C⁺, HLA-DP,DQ,DR⁻, or Programmed Death-1 Ligand (PDL1)⁺, as detectable by flow cytometry.

In certain embodiments, the placental adherent cells used in accordance with the methods of inducing lymphangiogenesis and methods of treatment described herein are, or are additionally, ABC-p⁺, as detectable by flow cytometry, or OCT-4⁺ (POU5F1⁺), e.g., as determinable by RT-PCR, wherein ABC-p is a placenta-specific ABC transporter protein (also known as breast cancer resistance protein (BCRP) and as mitoxantrone resistance protein (MXR)). In another embodiment, any of the placental adherent cells described herein are additionally SSEA3⁻ or SSEA4⁻, e.g., as determinable by flow cytometry, wherein SSEA3 is Stage Specific Embryonic Antigen 3, and SSEA4 is Stage Specific Embryonic Antigen 4. In another embodiment, any of the placental adherent cells described herein are additionally SSEA3⁻ and SSEA4⁻.

In certain embodiments, the placental adherent cells used in accordance with the methods of inducing lymphangiogenesis and methods of treatment described herein are additionally one or more of MHC-I⁺ (e.g., HLA-A,B,C⁺), MHC-II⁻ (e.g., HLA-DP,DQ,DR⁻) or HLA-G⁻. In another embodiment, any of the placental adherent cells described herein are additionally each of MHC-I⁺ (e.g., HLA-A,B,C⁺), MHC-II⁻ (e.g., HLA-DP,DQ,DR⁻) and HLA-G⁻.

In certain embodiments, the placental adherent cells used in accordance with the methods of inducing lymphangiogenesis and methods of treatment described herein are CD34⁻, CD10⁺, CD105⁺, CD200⁺ cells and are additionally one or more of CD29⁺, CD38⁻, CD44⁺, CD54⁺, CD80⁻, CD86⁻, SH3⁺ or SH4⁺. In another embodiment, the cells are additionally CD44⁺. In another embodiment, the CD34⁻, CD10⁺, CD105⁺, CD200⁺ placental adherent cells are additionally one or more of CD13⁺, CD29⁺, CD33⁺, CD38⁻, CD44⁺, CD45⁻, CD54⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁺), CD106/VCAM⁺, CD117⁻, CD144/VE-cadherin^{low}, CD184/CXCR4⁻, CD133⁻, OCT-4⁺, SSEA3⁻, SSEA4⁻, ABC-p⁺, KDR⁻ (VEGFR2), HLA-A,B,C⁺, HLA-DP,DQ,DR⁻, HLA-G⁻, or Programmed Death-1 Ligand (PDL1)⁺, or any combination thereof. In another embodiment, the CD34⁻, CD10⁺, CD105⁺, CD200⁺ placental adherent cells are additionally CD13⁺, CD29⁺, CD33⁺, CD38⁻, CD44⁺, CD45⁻, CD54/ICAM⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁺), CD106/VCAM⁺, CD117⁻, CD144/VE-cadherin^{dim}CD184/CXCR4⁻, CD133⁻, OCT-4⁺, SSEA3⁻, SSEA4⁻, ABC-p⁺, KDR⁻ (VEGFR2), HLA-A,B,C⁺, HLA-DP,DQ,DR⁻, HLA-G⁻, and Programmed Death-1 Ligand (PDL1)⁺.

In other embodiments of the methods disclosed herein, the isolated placental adherent cells are CD200⁺ and HLA-G⁻; CD73⁺, CD105⁺, and CD200⁺; CD200⁺ and OCT-4⁺; CD73⁺, CD105⁺ and HLA-G⁻; CD73⁺ and CD105⁺; or OCT-4⁺; or any combination thereof.

In certain embodiments of the methods disclosed herein, the isolated placental adherent cells are one or more of CD10⁺, CD29⁺, CD34⁻, CD38⁻, CD44⁺, CD45⁻, CD54⁺, CD90⁺, SH2⁺, SH3⁺, SH4⁺, SSEA3⁻, SSEA4⁻, OCT-4⁺, MHC-I⁺ or ABC-p⁺, where ABC-p is a placenta-specific ABC transporter protein (also known as breast cancer resistance protein (BCRP) and as mitoxantrone resistance protein (MXR)). In another embodiment, the isolated placental adherent cells are CD10⁺, CD29⁺, CD34⁻, CD38⁻, CD44⁺, CD45⁻, CD54⁺, CD90⁺, SH2⁺, SH3⁺, SH4⁺, SSEA3⁻, SSEA4⁻, and OCT-4⁺. In another embodiment, the isolated placental adherent cells are CD10⁺, CD29⁺, CD34⁻, CD38⁻, CD45⁻, CD54⁺, SH2⁺, SH3⁺, and SH4⁺. In another embodiment, the isolated placental adherent cells are CD10⁺, CD29⁺, CD34⁻, CD38⁻, CD45⁻, CD54⁺, SH2⁺, SH3⁺, SH4⁺ and OCT-4⁺. In another embodiment, the isolated placental adherent cells are CD10⁺, CD29⁺, CD34⁻, CD38⁻, CD44⁺, CD45⁻, CD54⁺, CD90⁺, MHC-1⁺, SH2⁺, SH3⁺, SH4⁺. In another embodiment, the isolated placental adherent cells are OCT-4⁺ and ABC-p⁺. In another embodiment, the isolated placental adherent cells are SH2⁺, SH3⁺, SH4⁺ and OCT-4⁺. In another embodiment, the isolated placental adherent cells are OCT-4⁺, CD34⁻, SSEA3⁻, and SSEA4⁻. In a specific embodiment, said OCT-4⁺, CD34⁻, SSEA3⁻, and SSEA4⁻ cells are additionally CD10⁺, CD29⁺, CD34⁻, CD44⁺, CD45⁻, CD54⁺, CD90⁺, SH2⁺, SH3⁺, and SH4⁺. In another embodiment, the isolated placental adherent cells are OCT-4⁺ and CD34⁻, and either SH3⁺ or SH4⁺. In another embodiment, the isolated placental adherent cells are CD34⁻ and either CD10⁺, CD29⁺, CD44⁺, CD54⁺, CD90⁺, or OCT-4⁺. In certain embodiments, the isolated placental adherent cells are CD10⁺, CD34⁻, CD105⁺ and CD200⁺.

In certain embodiments of any of the above characteristics of the placental adherent cells used in the methods described herein, expression of the cellular marker (e.g., cluster of differentiation or immunogenic marker) is determinable by flow cytometry. In certain other embodiments, expression of the cellular marker is determinable by RT-PCR.

In certain embodiments, the placental adherent cells used in accordance with the methods of inducing lymphangiogenesis and methods of treatment described herein, e.g., CD10⁺, CD34⁻, CD105⁺, CD200⁺ cells, express one or more genes at a detectably higher level than an equivalent number of bone marrow-derived mesenchymal stem cells, wherein said one or more genes are one or more of ACTG2, ADARB1, AMIGO2, ARTS-1, B4GALT6, BCHE, C11orf9, CD200, COL4A1, COL4A2, CPA4, DMD, DSC3, DSG2, ELOVL2, F2RL1, FLJ10781, GATA6, GPR126, GPRC5B, ICAM1, IER3, IGFBP7, IL1A, IL6, IL18, KRT18, KRT8, LIPG, LRAP, MATN2, MEST, NFE2L3, NUAK1, PCDH7, PDLIM3, PKP2, RTN1, SERPINB9, ST3GAL6, ST6GALNAC5, SLC12A8, TCF21, TGFB2, VTN, and ZC3H12A, and wherein said bone marrow-derived mesenchymal stem cells have undergone a number of passages in culture equivalent to the number of passages said isolated placental adherent cells have undergone. In certain embodiments, said expression of said one ore more genes is determined, e.g., by RT-PCR or microarray analysis, e.g, using a U133-A microarray (Affymetrix). In another embodiment, said isolated placental adherent cells express said one or more genes when cultured for, e.g., anywhere from about 3 to about 35 population doublings, in a medium comprising 60% DMEM-LG (e.g., from Gibco) and 40% MCDB-201 (e.g., from Sigma); 2% fetal calf serum (e.g., from Hyclone Labs.); 1× insulin-transferrin-selenium (ITS); 1× linoleic acid-bovine serum albumin (LA-BSA); 10⁻⁹ M dexamethasone (e.g., from Sigma); 10⁻⁴ M ascorbic acid 2-phosphate (e.g., from Sigma); epidermal growth factor 10 ng/mL (e.g., from R&D Systems); and platelet-derived growth factor (PDGF-BB) 10 ng/mL (e.g., from R&D Systems). In another embodiment, said isolated placental adherent cells express said one or more genes when cultured for from about 3 to about 35 population doublings in a medium comprising 60% DMEM-LG (e.g., from Gibco) and 40% MCDB-201 (e.g., from Sigma); 2% fetal calf serum (e.g., from Hyclone Labs.); 1× insulin-transferrin-selenium (ITS); 1× linoleic acid-bovine serum albumin (LA-BSA); 10⁻⁹ M dexamethasone (e.g., from Sigma); 10⁻⁴ M ascorbic acid 2-phosphate (Sigma); epidermal growth factor 10 ng/mL (e.g., from R&D Systems); and platelet-derived growth factor (PDGF-BB) 10 ng/mL (e.g., from R&D Systems).

In certain embodiments, the placental adherent cells used in accordance with the methods of inducing lymphangiogenesis and methods of treatment described herein express CD200 and ARTS1 (aminopeptidase regulator of type 1 tumor necrosis factor); ARTS-1 and LRAP (leukocyte-derived arginine aminopeptidase); IL6 (interleukin-6) and TGFB2 (transforming growth factor, beta 2); IL6 and KRT18 (keratin 18); IER3 (immediate early response 3), MEST (mesoderm specific transcript homolog) and TGFB2; CD200 and IER3; CD200 and IL6; CD200 and KRT18; CD200 and LRAP; CD200 and MEST; CD200 and NFE2L3 (nuclear factor (erythroid-derived 2)-like 3); or CD200 and TGFB2 at a detectably higher level than an equivalent number of bone marrow-derived mesenchymal stem cells (BM-MSCs) wherein said bone marrow-derived mesenchymal stem cells have undergone a number of passages in culture equivalent to the number of passages said isolated placental adherent cells have undergone. In other embodiments, the placental adherent cells express ARTS-1, CD200, IL6 and LRAP; ARTS-1, IL6, TGFB2, IER3, KRT18 and MEST; CD200, IER3, IL6, KRT18, LRAP, MEST, NFE2L3, and TGFB2; ARTS-1, CD200, IER3, IL6, KRT18, LRAP, MEST, NFE2L3, and TGFB2; or IER3, MEST and TGFB2 at a detectably higher level than an equivalent number of bone marrow-derived mesenchymal stem cells BM-MSCs, wherein said bone marrow-derived mesenchymal stem cells have undergone a number of passages in culture equivalent to the number of passages said isolated placental adherent cells have undergone.

When human cells are used, the gene designations throughout refer to human sequences, and, as is well known to persons of skill in the art, representative sequences can be found in literature, or in GenBank. Probes to the sequences can be determinable by sequences that are publicly-available, or through commercial sources, e.g., specific TAQMAN® probes or TAQMAN® Angiogenesis Array (Applied Biosystems, part no. 4378710).

In various embodiments, said isolated placental adherent cells useful in the methods disclosed herein, e.g., placental stem cells or placental multipotent cells, are contained within a population of cells, at least 10%, 15%, 20%, 25%, 30%, 35%, 40%, 45%, 50%, 55%, 60%, 65%, 70%, 75%, 80%, 85%, 90%, 95%, 98% or 99% of the cells of which are said isolated placental adherent cells. In certain other embodiments, the placental adherent cells in said population of cells are substantially free of cells having a maternal genotype; e.g., at least 40%, 45%, 50%, 55%, 60%, 65%, 70%, 75%, 80%, 85%, 90%, 95%, 98% or 99% of the placental adherent cells in said population have a fetal genotype, i.e., are fetal in origin. In certain other embodiments, the population of cells comprising said placental adherent cells are substantially free of cells having a maternal genotype; e.g., at least 40%, 45%, 50%, 55%, 60%, 65%, 70%, 75%, 80%, 85%, 90%, 95%, 98% or 99% of the cells in said population have a fetal genotype, i.e., are fetal in origin. In certain other embodiments, the population of cells comprising said placental adherent cells comprise cells having a maternal genotype; e.g., at least 10%, 15%, 20%, 25%, 30%, 35%, 40%, 45%, 50%, 55%, 60%, 65%, 70%, 75%, 80%, 85%, 90%, 95%, 98% or 99% of the cells in said population have a maternal genotype, i.e., are maternal in origin.

In an embodiment of any of the embodiments of placental adherent cells (e.g., placental adherent cells) herein, the placental adherent cells facilitate the formation of one or more embryoid-like bodies in a population of placental adherent cells comprising said isolated placental adherent cells when said population is cultured under conditions that allow the formation of an embryoid-like body e.g., culture under proliferation conditions).

In certain embodiments, the placental adherent cells useful in the methods provided herein do not express CD34, as detectable by immunolocalization, after exposure to 1 to 100 ng/mL VEGF for 4 to 21 days. In a specific embodiment, said placental adherent cells are adherent to tissue culture plastic. In another specific embodiment, said population of cells forms sprouts or tube-like structures when cultured in the presence of an angiogenic factor such as vascular endothelial growth factor (VEGF), epithelial growth factor (EGF), platelet derived growth factor (PDGF) or basic fibroblast growth factor (bFGF), e.g., on a substrate such as MATRIGEL™.

In certain embodiments, the placental adherent cells used in accordance with the methods of inducing lymphangiogenesis and methods of treatment described herein secrete one or more, or all, of VEGF, HGF, IL-8, MCP-3, FGF2, follistatin, G-CSF, EGF, ENA-78, GRO, IL-6, MCP-1, PDGF-BB, TIMP-2, uPAR, or galectin-1, e.g., into culture medium in which the cell, or cells, are grown. In another embodiment, the placental adherent cells express increased levels of CD202b, IL-8 and/or VEGF under hypoxic conditions (e.g., less than about 5% 02) compared to normoxic conditions (e.g., about 20% or about 21% 02).

In certain embodiments, the placental adherent cells used in accordance with the methods of inducing lymphangiogenesis and methods of treatment described herein can cause the formation of sprouts or tube-like structures in a population of endothelial cells in contact with said placental derived adherent cells. In a specific embodiment, the placental-derived angiogenic cells are co-cultured with human endothelial cells, forming sprouts or tube-like structures, or supporting the endothelial cell sprouts, e.g., when cultured in the presence of extracellular matrix proteins such as collagen type I and IV, and/or angiogenic factors such as vascular endothelial growth factor (VEGF), epithelial growth factor (EGF), platelet derived growth factor (PDGF) or basic fibroblast growth factor (bFGF), e.g., in or on a substrate such as placental collagen or MATRIGEL™ for at least 4 days.

In certain embodiments of the placental adherent cells used in the methods of inducing lymphangiogenesis and methods of treatment described herein are human.

In certain embodiments, the placental adherent cells used in accordance with the methods of inducing lymphangiogenesis and methods of treatment described herein are autologous to a recipient. In certain embodiments, the placental adherent cells used in accordance with the methods of inducing lymphangiogenesis and methods of treatment described herein are heterologous to a recipient.

Cell surface, molecular and genetic markers of placental adherent cells, useful in the methods provided herein, are described in detail in Section 5.3, below.

In certain embodiments, the placental adherent cells used in accordance with the methods of inducing lymphangiogenesis and methods of treatment described herein, said isolated placental adherent cells are administered once to said individual. In another specific embodiment, said isolated placental adherent cells are administered to said individual in two or more separate administrations. In another specific embodiment, said administering comprises administering between about 1×10⁴ and 1×10⁵ isolated placental adherent cells, e.g., placental adherent cells per kilogram of said individual. In another specific embodiment, said administering comprises administering between about 1×10⁵ and 1×10⁶ isolated placental adherent cells per kilogram of said individual. In another specific embodiment, said administering comprises administering between about 1×10⁶ and 1×10⁷ isolated placental adherent cells per kilogram of said individual. In another specific embodiment, said administering comprises administering between about 1×10⁷ and 1×10⁸ isolated placental adherent cells per kilogram of said individual. In other specific embodiments, said administering comprises administering between about 1×10⁶ and about 2×10⁶ isolated placental adherent cells per kilogram of said individual; between about 2×10⁶ and about 3×10⁶ isolated placental adherent cells per kilogram of said individual; between about 3×10⁶ and about 4×10⁶ isolated placental adherent cells per kilogram of said individual; between about 4×10⁶ and about 5×10⁶ isolated placental adherent cells per kilogram of said individual; between about 5×10⁶ and about 6×10⁶ isolated placental adherent cells per kilogram of said individual; between about 6×10⁶ and about 7×10⁶ isolated placental adherent cells per kilogram of said individual; between about 7×10⁶ and about 8×10⁶ isolated placental adherent cells per kilogram of said individual; between about 8×10⁶ and about 9×10⁶ isolated placental adherent cells per kilogram of said individual; or between about 9×10⁶ and about 1×10⁷ isolated placental adherent cells per kilogram of said individual. In another specific embodiment, said administering comprises administering between about 1×10⁷ and about 2×10⁷ isolated placental adherent cells per kilogram of said individual to said individual. In another specific embodiment, said administering comprises administering between about 1.3×10⁷ and about 1.5×10⁷ isolated placental adherent cells per kilogram of said individual to said individual. In another specific embodiment, said administering comprises administering up to about 3×10⁷ isolated placental adherent cells per kilogram of said individual to said individual. In a specific embodiment, said administering comprises administering between about 5×10⁶ and about 2×10⁷ isolated placental adherent cells to said individual. In another specific embodiment, said administering comprises administering about 150×10⁶ isolated placental adherent cells in about 20 milliliters of solution to said individual.

In a specific embodiment, said administering comprises administering between about 5×10⁶ and about 2×10⁷ isolated placental adherent cells to said individual, wherein said cells are contained in a solution comprising 10% dextran, e.g., dextran-40, 5% human serum albumin, and optionally an immunosuppressant.

In another specific embodiment, said administering comprises administering between about 5×10⁷ and 3×10⁹ isolated placental adherent cells intravenously. In specific embodiments, said administering comprises administering about 9×10⁸ isolated placental adherent cells or about 1.8×10⁹ isolated placental adherent cells intravenously. In another specific embodiment, said administering comprises administering between about 5×10⁶ and 1×10⁸ isolated placental adherent cells subcutaneously. In another specific embodiment, said administering comprises administering about 9×10⁶ isolated placental adherent cells subcutaneously.

The placental adherent cells used in the methods provided herein can, in certain embodiments, be genetically engineered to produce one or more proteins that facilitate angiogenesis. In certain embodiments, said proteins that facilitate angiogenesis are one or more of hepatocyte growth factor (HGF), vascular endothelial growth factor (VEGF) (e.g., VEGFD), fibroblast growth factor (FGF) (e.g., FGF2), angiogenin (ANG), epidermal growth factor (EGF), epithelial-neutrophil-activating protein 78 (ENA-78), follistatin, granulocyte colony-stimulating factor (G-CSF), growth-regulated ongogene protein (GRO), interleukin-6 (IL-6), IL-8, leptin, monocyte chemotactic protein-1 (MCP-1), MCP-3, platelet-derived growth factor submunit B (PDGFB), rantes, transforming growth factor beta 1 (TGF-01), thrombopoitein (Tpo), tissue inhibitor of metalloproteinases 1 (TIMP1), TIMP2, and/or urokinase plasminogen activator receptor (uPAR).

### 4. BRIEF DESCRIPTION OF THE FIGURES

FIG. 1 shows the secretion of selected angiogenic proteins by placental derived adherent cells.

FIG. 2 shows the angiogenic effect of placental derived adherent cells conditioned medium on Human Endothelial Cell (HUVEC) tube formation.

FIG. 3 shows the angiogenic effect of placental derived adherent cells conditioned medium on Human Endothelial Cell migration.

FIG. 4 shows the effect of placental derived adherent cell-conditioned medium on Human Endothelial Cell proliferation.

FIG. 5 shows tube formation of HUVECs and placental derived adherent cells.

FIG. 6 shows the secretion of VEGF and IL-8 by placental derived adherent cells under hypoxic and normoxic conditions.

FIG. 7 shows positive effect of placental adherent cell on angiogenesis in a chick chorioallantois angiogenesis model. bFGF: basic fibroblast growth factor (positive control). MDAMB231: Angiogenic breast cancer cell line (positive control). Y axis: Degree of blood vessel formation.

FIG. 8 shows positive effect of placental adherent cell-conditioned medium (supernatants) on angiogenesis in a chick chorioallantois angiogenesis model. bFGF: basic fibroblast growth factor (positive control). MDAMB231: Angiogenic breast cancer cell line (positive control). Y axis: Degree of blood vessel formation.

FIG. 9: CD34⁻, CD10⁺, CD105⁺, CD200⁺ placental adherent cells enhance arteriogenesis in a unilateral hind limb ischemia (HLI) model in db/db mice. FIG. 9A: Laser Doppler analysis 14-, 28- and 42-days post-ligation (n=9). FIG. 9B: Quantification of CD31⁺ blood vessels in the thigh 42 days post-HLI (n=4). FIG. 9C: Quantification of αSMA⁺ blood vessels in the thigh 42 days post-HLI (n=4). FIG. 9D: Size distribution of αSMA⁺ blood vessels in the thigh 42 days post-HLI (n=4). FIG. 9E: Semi-quantitative assessment of limb function 4-, 28- and 42-days post-HLI (n=9). All graphs show mean s.e.m. *, p<0.05; **, p<0.01. N=number of animals.

FIG. 10: CD34⁻, CD10⁺, CD105⁺, CD200⁺ placental adherent cells promote M2 macrophage differentiation in a unilateral HLI model in db/db mice. FIG. 10A: Quantification of CD68⁺ macrophages 7-, 14- and 28-days post-HLI (n=4); FIG. 10B: Quantification of CD68+ Arg1+ macrophages 7-, 14- and 28-days post-HLI (n=4). FIG. 10C-E: CD206 (10C), HLA-I (10D), and CD80 (10E) surface phenotype of human macrophages before and after co-culture with LPS, IL-4 and placental adherent cells for 48 hours. All graphs show mean s.e.m. *, p<0.05; **, p<0.01; *** p<0.005; **** p<0.001. N=number of animals.

FIG. 11: CD34⁻, CD10⁺, CD105⁺, CD200⁺ placental adherent cell-mediated arteriogenesis is T cell-dependent. FIG. 11A: Laser Doppler analysis 14-, 28- and 42-days post-HLI in wild-type (WT) and nude Balb/c mice an HLI model (WT: n=15; nude: n=9). FIG. 11B: Quantification of CD31⁺ blood vessels in the thigh 42 days post-HLI (n=5). FIG. 11C Quantification of αSMA⁺ blood vessels in the thigh 42 days post-HLI (n=5). FIG. 11D: Laser Doppler analysis 7-, 14-, 21-, 28- and 35-days post-HLI in T cell-reconstituted nude Balb/c HLI model mice (n=12). All graphs show mean±s.e.m. *, p<0.05; **, p<0.01; *** p<0.005; ****, p<0.001. N=number of animals.

FIG. 12: CD34⁻, CD10⁺, CD105⁺, CD200⁺ placental adherent cell-induced M2 macrophage differentiation is T cell-dependent. FIG. 12A: Quantification of CD68⁺ total macrophages in the thigh 7 days post-HLI (n=5). FIG. 12B: Quantification of CD68⁺ Arg1+M2 macrophages in the thigh 7 days post-HLI (n=5). All graphs show mean s.e.m. p<0.05; **, p<0.01; ***, p<0.005. N=number of animals.

FIG. 13: CD34⁻, CD10⁺, CD105⁺, CD200⁺ placental adherent cells have a systemic arteriogenic effect in a bilateral HLI model in db/db mice. FIG. 13A: Laser Doppler analysis in first and second injured hind-limbs (n=13). FIG. 13B: Quantification of blood vessels based on X-ray angiography (n=5). FIG. 13C-D: Representative X-ray angiographs. FIG. 13E: Quantification of CD31⁺ blood vessels in the thigh 64 days post first injury (vehicle: n=9; placental adherent cells: n=13). FIG. 13F: Quantification of αSMA⁺ blood vessels in the thigh 64 days post first injury (vehicle: n=9; placental adherent cells: n=13). FIG. 13G: Size distribution of αSMA⁺ large blood vessels in the thigh 64 days post first injury (vehicle: n=9; placental adherent cells: n=13). FIG. 13H: Laser Doppler analysis in first and second injured hind-limbs (n=18). FIG. 13I: Quantification of blood vessels based on X-ray angiography (n=5). FIG. 13J: Quantification of CD68⁺ Arg1+ macrophages 7 days post second injury (n=4). All graphs show mean±s.e.m. *, p<0.05; **, p<0.01; ***, p<0.005. N=number of animals.

### 5. DETAILED DESCRIPTION

### 5.1 Definitions

As used herein, the term “about,” when referring to a stated numeric value, indicates a value within plus or minus 10% of the stated numeric value.

As used herein, the term “angiogenic,” in reference to the placental derived adherent cells described herein, means that the cells can form vessels or vessel-like sprouts, or that the cells can promote angiogenesis (e.g., the formation of vessels or vessel-like structures) in another population of cells, e.g., endothelial cells.

As used herein, the term “angiogenesis” refers to the process of blood vessel formation that includes, but is not limited to, endothelial cell activation, migration, proliferation, matrix remodeling and cell stabilization.

As used herein, the term “derived” means isolated from or otherwise purified. For example, placental derived adherent cells are isolated from placenta. The term “derived” encompasses cells that are cultured from cells isolated directly from a tissue, e.g., the placenta, and cells cultured or expanded from primary isolates.

As used herein, “immunolocalization” means the detection of a compound, e.g., a cellular marker, using an immune protein, e.g., an antibody or fragment thereof in, for example, flow cytometry, fluorescence-activated cell sorting, magnetic cell sorting, in situ hybridization, immunohistochemistry, or the like.

As used herein, the term “SH2” refers to an antibody that binds an epitope on the cellular marker CD105. Thus, cells that are referred to as SH2⁺ are CD105⁺.

As used herein, the terms “SH3” and “SH4′” refer to antibodies that bind epitopes present on the cellular marker CD73. Thus, cells that are referred to as SH3⁺ and/or SH4⁺ are CD73⁺.

A placenta has the genotype of the fetus that develops within it, but is also in close physical contact with maternal tissues during gestation. As such, as used herein, the term “fetal genotype” means the genotype of the fetus, e.g., the genotype of the fetus associated with the placenta from which particular isolated placental adherent cells, as described herein, are obtained, as opposed to the genotype of the mother that carried the fetus. As used herein, the term “maternal genotype” means the genotype of the mother that carried the fetus, e.g., the fetus associated with the placenta from which particular isolated placental adherent cells, as described herein, are obtained.

As used herein, the term “isolated cell,” e.g., “isolated placental cell,” “isolated placental stem cell,” and the like, means a cell that is substantially separated from other, different cells of the tissue, e.g., placenta, from which the stem cell is derived. A cell is “isolated” if at least 50%, 60%, 70%, 80%, 90%, 95%, or at least 99% of the cells, e.g., non-stem cells, with which the stem cell is naturally associated, or stem cells displaying a different marker profile, are removed from the stem cell, e.g., during collection and/or culture of the stem cell.

As used herein, “multipotent,” when referring to a cell, means that the cell has the ability to differentiate into some, but not necessarily all, types of cells of the body, or into cells having characteristics of some, but not all, types of cells of the body, or into cells of one or more of the three germ layers. In certain embodiments, for example, an isolated placental cell (placental adherent cell), as described in Section 5.3, below, that has the capacity to differentiate into a cell having characteristics of neurogenic, chondrogenic and/or osteogenic cells is a multipotent cell.

As used herein, the term “population of isolated cells” means a population of cells that is substantially separated from other cells of a tissue, e.g., placenta, from which the population of cells is derived.

As used herein, the term “placental adherent cells” means tissue culture plastic-adherent cells that are isolated from a mammalian placenta either as primary isolates or cultured cells, regardless of the number of passages after a primary culture, e.g., the placental adherent cells described in Section 5.3, below. The term “placental adherent cells” as used herein does not, however, refer to, and the placental adherent cells used in the methods provided herein are not, trophoblasts, cytotrophoblasts, syncitiotrophoblasts, angioblasts, hemangioblasts, embryonic germ cells, embryonic stem cells, cells obtained from an inner cell mass of a blastocyst, or cells obtained from a gonadal ridge of a late embryo, e.g., embryonic germ cells. The placental adherent cells, e.g., placental adherent cells, described herein are also not the amnion-derived adherent cells described in U.S. Pat. No. 8,637,409, entitled “Amnion Derived Angiogenic Cells,” the disclosure of which is hereby incorporated by reference in its entirety. A cell is considered a “stem cell” if the cell displays attributes of a stem cell, e.g., a marker or gene expression profile associated with one or more types of stem cells; the ability to replicate at least 10-40 times in culture, and the ability to differentiate into cells displaying characteristics of differentiated cells of one or more of the three germ layers. Unless otherwise noted herein, the term “placental” includes the umbilical cord. The isolated placental adherent cells disclosed herein, in certain embodiments, differentiate in vitro under differentiating conditions, differentiate in vivo, or both.

As used herein, a placental cell is “positive” for a particular marker when that marker is detectable above background. Detection of a particular marker can, for example, be accomplished either by use of antibodies, or by oligonucleotide probes or primers based on the sequence of the gene or mRNA encoding the marker. For example, a placental cell is positive for, e.g., CD73 because CD73 is detectable on placental adherent cells in an amount detectably greater than background (in comparison to, e.g., an isotype control). A cell is also positive for a marker when that marker can be used to distinguish the cell from at least one other cell type, or can be used to select or isolate the cell when present or expressed by the cell. In the context of, e.g., antibody-mediated detection, “positive,” as an indication a particular cell surface marker is present, means that the marker is detectable using an antibody, e.g., a fluorescently-labeled antibody, specific for that marker; “positive” also refers to a cell exhibiting the marker in an amount that produces a signal, e.g., in a cytometer, that is detectably above background. For example, a cell is “CD200⁺” where the cell is detectably labeled with an antibody specific to CD200, and the signal from the antibody is detectably higher than that of a control (e.g., background or an isotype control). Conversely, “negative” in the same context means that the cell surface marker is not detectable using an antibody specific for that marker compared a control (e.g., background or an isotype control). For example, a cell is “CD34⁻” where the cell is not reproducibly detectably labeled with an antibody specific to CD34 to a greater degree than a control (e.g., background or an isotype control). Markers not detected, or not detectable, using antibodies are determined to be positive or negative in a similar manner, using an appropriate control. For example, a cell or population of cells can be determined to be OCT-4⁺ if the amount of OCT-4 RNA detected in RNA from the cell or population of cells is detectably greater than background as determined, e.g., by a method of detecting RNA such as RT-PCR, slot blots, etc. Unless otherwise noted herein, cluster of differentiation (“CD”) markers are detected using antibodies. In certain embodiments, OCT-4 is determined to be present, and a cell is “OCT-4⁺” if OCT-4 mRNA is detectable using RT-PCR.

As used herein, the designation “low,” when referring to the expression of a marker detectable in flow cytometry, means that the marker is expressed by fewer than 10% of cells tested, or that fluorescence attributable to the marker in, e.g., flow cytometry, is less than 1 log above background.

As used herein, “treat” encompasses the remediation of, improvement of, lessening of the severity of, or reduction in the time course of, a disease, disorder or condition, or any parameter or symptom thereof.

### 5.2 Treatment of Lymphedema and Diseases or Disorders Caused by Lymphedema

Provided herein are methods of treating lymphedema, or diseases or disorders caused by lymphedema, comprising administering an effective amount of adherent placental adherent cells, e.g., the placental adherent cells described in Section 5.3, below. Placental adherent cells are able to support growth of endothelial cells and endothelial cells populations, and epithelial cells and epithelial cell populations, in the lymphatic system.

In certain embodiments, the method comprises administering placental adherent cells to said individual in an amount and for a time sufficient for an improvement of one or more of lymphedema-related or lymphedema-caused symptoms or sequlae such as swelling of part or all of one or both legs, swelling of part or all of one or both arms, heaviness or tightness in an arm or leg, nontender pitted edema of a leg or arm, restricted motion or a sensation of restricted motion in an arm or leg, aching or discomfort in an arm or leg, infection in or below the skin, cellulitis, lymphangitis, lymphorrhea, impetigo, erythema of the skin of an affected arm or leg, a positive stemmer's sign, or hardening and/or thickening of the skin of an affected arm or leg, as compared to the individual prior to administration of said placental adherent cells. In certain embodiments, the lymphedema is Stage 0, Stage 1, Stage 2, or Stage 3 lymphedema. In certain other embodiments, the lymphedema is adjunct to lipedema.

Stages of lymphedema: Lymphedema generally proceeds in four stages. In stage 0, protein begins to accumulate under the skin, causing local accumulation of water and an accompanying sensation of heaviness or fatigue in the affected limb or region. In Stage 1 lymphedema, the foot and or hands (part of the limbs furthest from the body) begin to swell and take on a puffy appearance; impressions or “pitting” may be evident; the skin also retains a small pit when pressed with a finger. This is known as “pitting edema”. At this stage, edema may be reduced at night as compared to the day, and elevation of the affected limb can reduce the edema. In Stage 2 lymphedema, swelling in the limb takes on a spongy consistency, and pitting is reduced or absent compared to Stage 1. The lymphedema does not respond to elevation. The swelling is now associated with or caused by fibrosis and scar tissue under the skin. In Stage 3, also known as lymphostatic elephantiasis, the affected limb is quite swollen. The skin typically becomes dry and flaky, with hyperkeratosis and hardening developing. Fluid may leak from the skin, fluid-filled blisters may form, and skin infections are common.

Administration of adherent placental adherent cells, or therapeutic compositions comprising such cells, to an individual with lymphedema in need thereof, can be accomplished, e.g., by transplantation, implantation (e.g., of the cells themselves or the cells as part of a matrix-cell combination), injection (e.g., directly to the site of the disease or condition, for example, directly to an ischemic site in the heart of an individual who has had a myocardial infarction), infusion, delivery via catheter, or any other means known in the art for providing cell therapy.

In one embodiment, the therapeutic cell compositions are provided to an individual in need thereof, for example, by injection into one or more sites in the individual. In a specific embodiment, the therapeutic cell compositions are provided by intracardiac injection, e.g., to an ischemic area in the heart. In other specific embodiments, the cells are injected onto the surface of the heart, into an adjacent area, or even to a more remote area. In preferred embodiments, the cells can home to the diseased or injured area.

An individual having lymphedema can be administered placental adherent cells at any time the cells would be therapeutically beneficial. In certain embodiments, for example, the placental adherent cells or therapeutic compositions of the invention are administered within 1, 2, 3, 4, 5, 6, 7, 8, 9, 10, 11, 12, 13, 14, 15, 16, 17, 18, 19, 20, 21, 22, 23, or 24 hours, or 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or 1, 2, 3, 4, 5, 6, 7, 8, 9, 10, 11, 12 months, or 1, 2, 3, 4, 5, 6, 7, 8, 9, 10, 11, 12 years of diagnosis of lymphedema.

Also provided herein are kits for use in the treatment of lymphedema. The kits provide a therapeutic cell composition comprising placental adherent cells, which can be prepared in a pharmaceutically acceptable form, for example by mixing with a pharmaceutically acceptable carrier, and an applicator, along with instructions for use. Ideally the kit can be used in the field or at bedside, for example in a physician's office.

In a specific embodiment, the methods of treatment provided herein comprise administering placental adherent cells, e.g., a therapeutic composition comprising the placental adherent cells, to a patient with lymphedema, wherein the therapeutic cell composition is administered as a matrix-cell complex. In certain embodiments, the matrix is a scaffold, preferably bioabsorbable, comprising at least the cells.

To this end, further provided herein are populations of placental adherent cells contacted with, e.g., incubated or cultured in the presence of, one or more factors that stimulate stem or progenitor cell differentiation along a lymphangiogenic pathway. Such factors include, but are not limited to factors, such as growth factors, chemokines, cytokines, cellular products, demethylating agents, and other factors which are now known or later determined to stimulate differentiation, for example of stem cells, along lymphangiogenic pathways or lineages.

In certain embodiments, placental adherent cells may be differentiated along lymphangiogenic pathways or lineages in vitro by culture of the cells in the presence of factors comprising at least one of a demethylation agent, a BMP (bone morphogenetic protein), FGF (fibroblast growth factor), Wnt factor protein, Hedgehog protein, and/or an anti-Wnt factor.

Placental adherent cells, and populations of such cells, can be provided therapeutically or prophylactically to an individual having lymphedema, or a symptom or sequela of lymphedema.

In certain embodiments, the individual is administered a therapeutically effective amount of placental adherent cells, e.g., in a population of cells that comprise the placental adherent cells. In a specific embodiment, the population comprises about 50% placental adherent cells. In another specific embodiment, the population is a substantially homogeneous population of placental adherent cells. In other embodiments the population comprises at least about 5%, 10%, 15%, 20%, 25%, 30%, 35%, 40%, 45%, 50%, 55%, 60%, 65%, 70%, 75%, 80%, 85% 90%, 95%, 98% or 99% placental adherent cells.

The placental adherent cells may be administered to an individual in the form of a therapeutic composition comprising the cells and another therapeutic agent, such as insulin-like growth factor (IGF), platelet-derived growth factor (PDGF), epidermal growth factor (EGF), fibroblast growth factor (FGF), vascular endothelial growth factor (VEGF), hepatocyte growth factor (HGF), interleukin 18 (IL-8), an antithrombogenic agent (e.g., heparin, heparin derivatives, urokinase, or PPack (dextrophenylalanine proline arginine chloromethylketone), an antithrombin compound, a platelet receptor antagonist, an antithrombin antibody, an anti-platelet receptor antibody, aspirin, dipyridamole, protamine, hirudin, a prostaglandin inhibitor, and/or a platelet inhibitor), an antiapoptotic agent (e.g., erythropoietin (Epo), an Epo derivative or analog, or their salts, thrombopoietin (Tpo), IGF-I, IGF-II, hepatocyte growth factor (HGF), or a caspase inhibitor), an anti-inflammatory agent (e.g., a p38 MAP kinase inhibitor, a statin, in IL-6 inhibitor, an IL-1 inhibitor, Pemirolast, Tranilast, Remicade, Sirolimus, and/or a nonsteroidal anti-inflammatory compound (e.g., acetylsalicylic acid, ibuprofen, Tepoxalin, Tolmetin, or Suprofen)), an immunosuppressive or immunomodulatory agent (e.g., a calcineurin inhibitor, for example cyclosporine, Tacrolimus, an mTOR inhibitor such as Sirolimus or Everolimus; an anti-proliferative such as azathioprine and/or mycophenolate mofetil; a corticosteroid, e.g., prednisolone or hydrocortisone; an antibody such as a monoclonal anti-IL-2Rα receptor antibody, Basiliximab, Daclizuma, polyclonal anti-T-cell antibodies such as anti-thymocyte globulin (ATG), anti-lymphocyte globulin (ALG), and/or the monoclonal anti-T cell antibody OKT3), and/or an antioxidant (e.g., probucol; vitamins A, C, and/or E, coenzyme Q-10, glutathione, L cysteine, N-acetylcysteine, or an antioxidant derivative, analog or salt of any of the foregoing). In certain embodiments, therapeutic compositions comprising the placental adherent cells further comprise one or more additional cell types, e.g., adult cells (for example, fibroblasts or endodermal cells), stem cells and/or progenitor cells. Such therapeutic agents and/or one or more additional types of cells can be administered to an individual in need thereof individually or in combinations or two or more such compounds or agents.

In certain embodiments, the individual to be treated is a mammal. In a specific embodiment the individual to be treated is a human.

### 5.3 Isolated Placental Adherent Cells and Isolated Placental Cell Populations

The isolated placental adherent cells, e.g., placental adherent cells, useful in the treatment of individuals having a circulatory disease or disorder, are cells, obtainable from a placenta or part thereof, that adhere to a tissue culture substrate and have characteristics of multipotent cells or stem cells, but are not trophoblasts. In certain embodiments, the isolated placental adherent cells useful in the methods disclosed herein have the capacity to differentiate into non-placental cell types.

The isolated placental adherent cells useful in the methods disclosed herein can be either fetal or maternal in origin (that is, can have the genotype of either the fetus or mother, respectively). Preferably, the isolated placental adherent cells and populations of isolated placental adherent cells are fetal in origin. As used herein, the phrase “fetal in origin” or “non-maternal in origin” indicates that the isolated placental adherent cells or populations of isolated placental adherent cells are obtained from the umbilical cord or placental structures associated with the fetus, i.e., that have the fetal genotype. As used herein, the phrase “maternal in origin” indicates that the cells or populations of cells are obtained from a placental structures associated with the mother, e.g., which have the maternal genotype. Isolated placental adherent cells, e.g., placental adherent cells, or populations of cells comprising the isolated placental adherent cells, can comprise isolated placental adherent cells that are solely fetal or maternal in origin, or can comprise a mixed population of isolated placental adherent cells of both fetal and maternal origin. The isolated placental adherent cells, and populations of cells comprising the isolated placental adherent cells, can be identified and selected by the morphological, marker, and culture characteristics discussed below. In certain embodiments, any of the placental adherent cells, e.g., placental stem cells or placental multipotent cells described herein, are autologous to a recipient, e.g., an individual who has a disease or disorder of the circulatory system. In certain other embodiments, any of the placental adherent cells, e.g., placental stem cells or placental multipotent cells described herein, are heterologous to a recipient, e.g., an individual who has a disease or disorder of the circulatory system.

### 5.3.1 Physical and Morphological Characteristics

The isolated placental adherent cells described herein, when cultured in primary cultures or in cell culture, adhere to the tissue culture substrate, e.g., tissue culture container surface (e.g., tissue culture plastic), or to a tissue culture surface coated with extracellular matrix or ligands such as laminin, collagen (e.g., native or denatured), gelatin, fibronectin, ornithine, vitronectin, and extracellular membrane protein (e.g., MATRIGEL™ (BD Discovery Labware, Bedford, Mass.)). The isolated placental adherent cells in culture assume a generally fibroblastoid, stellate appearance, with a number of cytoplasmic processes extending from the central cell body. The placental adherent cells are, however, morphologically distinguishable from fibroblasts cultured under the same conditions, as the isolated placental adherent cells exhibit a greater number of such processes than do fibroblasts. Morphologically, isolated placental adherent cells are also distinguishable from hematopoietic stem cells, which generally assume a more rounded, or cobblestone, morphology in culture.

In certain embodiments, the isolated placental adherent cells useful in the methods disclosed herein, when cultured in a growth medium, develop embryoid-like bodies. Embryoid-like bodies are noncontiguous clumps of cells that can grow on top of an adherent layer of proliferating isolated placental adherent cells. The term “embryoid-like” is used because the clumps of cells resemble embryoid bodies, clumps of cells that grow from cultures of embryonic stem cells. Growth medium in which embryoid-like bodies can develop in a proliferating culture of isolated placental adherent cells includes medium comprising, e.g., DMEM-LG (e.g., from Gibco); 2% fetal calf serum (e.g., from Hyclone Labs.); 1× insulin-transferrin-selenium (ITS); 1× linoleic acid-bovine serum albumin (LA-BSA); 10⁻⁹M dexamethasone (e.g., from Sigma); 10⁻⁴M ascorbic acid 2-phosphate (e.g., from Sigma); epidermal growth factor 10 ng/mL (e.g., from R&D Systems); and platelet-derived growth factor (PDGF-BB) 10 ng/mL (e.g., from R&D Systems).

### 5.3.2 Cell Surface, Molecular and Genetic Markers

The isolated placental adherent cells, e.g., isolated multipotent placental adherent cells or isolated placental stem cells, and populations of such isolated placental adherent cells, useful in the methods of disclosed herein, e.g., the methods of treatment of a disease or disorder of the circulatory system, are tissue culture plastic-adherent human placental adherent cells that have characteristics of multipotent cells or stem cells, and express a plurality of markers that can be used to identify and/or isolate the cells, or populations of cells that comprise the stem cells. In certain embodiments, the placental adherent cells are lymphangiogenic, e.g., in vitro or in vivo. The isolated placental adherent cells, and placental cell populations described herein (that is, two or more isolated placental adherent cells) include placental adherent cells and placental cell-containing cell populations obtained directly from the placenta, or any part thereof (e.g., chorion, placental cotyledons, or the like). Isolated placental cell populations also include populations of (that is, two or more) isolated placental adherent cells in culture, and a population in a container, e.g., a bag. The isolated placental adherent cells described herein are not bone marrow-derived mesenchymal cells, adipose-derived mesenchymal stem cells, or mesenchymal cells obtained from umbilical cord blood, placental blood, or peripheral blood. Placental cells, e.g., placental multipotent cells and placental adherent cells, useful in the methods and compositions described herein are described herein and, e.g., in U.S. Pat. Nos. 7,311,904; 7,311,905; and 7,468,276; and in U.S. Patent Application Publication No. 2007/0275362, the disclosures of which are hereby incorporated by reference in their entireties.

In certain embodiments, the isolated placental adherent cells are isolated placental multipotent cells. In one embodiment, the isolated placental adherent cells are CD34⁻, CD10⁺, CD105⁺ and CD200⁺ as detectable by flow cytometry. In another specific embodiment, the isolated CD34⁻, CD10⁺, CD105⁺ and CD200⁺ placental adherent cells have the potential to differentiate into cells of a neural phenotype, cells of an osteogenic phenotype, and/or cells of a chondrogenic phenotype. In another specific embodiment, isolated CD34⁻, CD10⁺, CD105⁺ placental adherent cells are additionally CD200⁺. In another specific embodiment, isolated CD200⁺ placental adherent cells are additionally CD45⁻ or CD90⁺, as detectable by flow cytometry. In another specific embodiment, isolated CD200⁺ placental adherent cells are additionally CD45⁻ and CD90⁺, as detectable by flow cytometry. In another specific embodiment, the isolated CD34⁻, CD10⁺, CD105⁺ and CD200⁺ placental adherent cells are additionally CD45⁻ or CD90⁺, as detectable by flow cytometry. In another specific embodiment, the isolated CD34⁻, CD10⁺, CD105⁺ and CD200⁺ placental adherent cells are additionally CD45⁻ and CD90⁺, as detectable by flow cytometry, i.e., the cells are CD34⁻, CD10⁺, CD105⁺ and CD200⁺, CD105⁺ and CD200⁺. In another specific embodiment, said CD34⁻, CD10⁺, CD105⁺ and CD200⁺, CD105⁺, CD200⁺ cells are additionally CD80⁻ and CD86⁻.

In certain embodiments, said placental adherent cells are CD34⁻, CD10⁺, CD105⁺ and CD200⁺, and one or more of CD38⁻, CD45⁻, CD80⁻, CD86⁻, CD133⁻, HLA-DR,DP,DQ⁻, SSEA3⁻, SSEA4⁻, CD29⁺, CD44⁺, CD73⁺, CD90⁺, CD105⁺, HLA-A,B,C⁺, PDL1⁺, ABC-p⁺, and/or OCT-4⁺, as detectable by flow cytometry or RT-PCR. In other embodiments, any of the CD34⁻, CD10⁺, CD105⁺ cells described above are additionally one or more of CD29⁺, CD38⁻, CD44⁺, CD54⁺, SH3⁺ or SH4⁺. In another specific embodiment, the cells are additionally CD44⁺. In another specific embodiment of any of the isolated CD34⁻, CD10⁺, CD105⁺ and CD200⁺ placental adherent cells above, the cells are additionally one or more of CD117⁻, CD133⁻, KDR⁻ (VEGFR2⁻), HLA-A,B,C⁺, HLA-DP,DQ,DR⁻, or Programmed Death-1 Ligand (PDL1)⁺, or any combination thereof.

In another embodiment, the CD34⁻, CD10⁺, CD105⁺ placental adherent cells are additionally one or more of CD13⁺, CD29⁺, CD33⁺, CD38⁻, CD44⁺, CD45⁻, CD54⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁺), CD106/VCAM⁺, CD117⁻, CD144/VE-cadherin^{low}, CD184/CXCR4⁻, CD200⁺, CD133⁻, OCT-4⁺, SSEA3⁻, SSEA4⁻, ABC-p⁺, KDR⁻ (VEGFR2⁻), HLA-A,B,C⁺, HLA-DP,DQ,DR⁻, HLA-G⁻, or Programmed Death-1 Ligand (PDL1)⁺, or any combination thereof. In a other embodiment, the CD34⁻, CD10⁺, CD105⁺ cells are additionally CD13⁺, CD29⁺, CD33⁺, CD38⁻, CD44⁺, CD45⁻, CD54/ICAM⁺, CD62E⁻, CD62L⁻, CD62P⁻, SH3⁺ (CD73⁺), SH4+(CD73⁺), CD80⁻, CD86⁻, CD90⁺, SH2⁺ (CD105⁺), CD106/VCAM⁺, CD117⁻, CD144/VE-cadherin^{low}, CD184/CXCR4⁻, CD200⁺, CD133⁻, OCT-4⁺, SSEA4⁻, SSEA4⁻, ABC-p⁺, KDR⁻ (VEGFR2⁻), HLA-A,B,C⁺, HLA-DP,DQ,DR⁻, HLA-G⁻, and Programmed Death-1 Ligand (PDL1)⁺.

In another specific embodiment, any of the placental adherent cells described herein are additionally ABC-p⁺, as detectable by flow cytometry, or OCT-4⁺ (POU5F1⁺), as determinable by RT-PCR, wherein ABC-p is a placenta-specific ABC transporter protein (also known as breast cancer resistance protein (BCRP) and as mitoxantrone resistance protein (MXR)), and OCT-4 is the Octamer-4 protein (POU5F1). In another specific embodiment, any of the placental adherent cells described herein are additionally SSEA3⁻ or SSEA4⁻, as determinable by flow cytometry, wherein SSEA3 is Stage Specific Embryonic Antigen 3, and SSEA4 is Stage Specific Embryonic Antigen 4. In another specific embodiment, any of the placental adherent cells described herein are additionally SSEA3⁻ and SSEA4⁻.

In another specific embodiment, any of the placental adherent cells described herein are additionally one or more of MHC-I⁺ (e.g., HLA-A,B,C⁺), MHC-II⁻ (e.g., HLA-DP,DQ,DR⁻) or HLA-G⁻. In another specific embodiment, any of the placental adherent cells described herein are additionally one or more of MHC-I⁺ (e.g., HLA-A,B,C⁺), MHC-II⁻ (e.g., HLA-DP,DQ,DR⁻) and HLA-G⁻.

Also provided herein are populations of the isolated placental adherent cells, or populations of cells, e.g., populations of placental adherent cells, comprising, e.g., that are enriched for, the isolated placental adherent cells, that are useful in the methods and compositions disclosed herein. Preferred populations of cells comprising the isolated placental adherent cells, wherein the populations of cells comprise, e.g., at least 10%, 15%, 20%, 25%, 30%, 35%, 40%, 45%, 50%, 55%, 60%, 65%, 70%, 75%, 80%, 85%, 90%, 95% or 98% isolated CD10⁺, CD105⁺ and CD34⁻ placental adherent cells; that is, at least 10%, 15%, 20%, 25%, 30%, 35%, 40%, 45%, 50%, 55%, 60%, 65%, 70%, 75%, 80%, 85%, 90%, 95% or 98% of cells in said population are isolated CD10⁺, CD105⁺ and CD34⁻ placental adherent cells. In a specific embodiment, the isolated CD34⁻, CD10⁺, CD105⁺ placental adherent cells are additionally CD200⁺. In another specific embodiment, the isolated CD34⁻, CD10⁺, CD105⁺, CD200⁺ placental adherent cells are additionally CD90⁺ or CD45⁻, as detectable by flow cytometry. In another specific embodiment, the isolated CD34⁻, CD10⁺, CD105⁺, CD200⁺ placental adherent cells are additionally CD90⁺ and CD45⁻, as detectable by flow cytometry. In another specific embodiment, any of the isolated CD34⁻, CD10⁺, CD105⁺ placental adherent cells described above are additionally one or more of CD29⁺, CD38⁻, CD44⁺, CD54⁺, SH3⁺ or SH4⁺. In another specific embodiment, the isolated CD34⁻, CD10⁺, CD105⁺ placental adherent cells, or isolated CD34⁻, CD10⁺, CD105⁺, CD200⁺ placental adherent cells, are additionally CD44⁺. In a specific embodiment of any of the populations of cells comprising isolated CD34⁻, CD10⁺, CD105⁺ placental adherent cells above, the isolated placental adherent cells are additionally one or more of CD13⁺, CD29⁺, CD33⁺, CD38⁻, CD44⁺, CD45⁻, CD54⁺, CD62E⁻, CD62L⁻, CD62P⁻, SH3⁺ (CD73⁺), SH4+(CD73⁺), CD80⁻, CD86⁻, CD90⁺, SH2⁺ (CD105⁺), CD106/VCAM⁺, CD117, CD144/VE-cadherin^{low}, CD184/CXCR4⁻, CD200⁺, CD133, OCT-4⁺, SSEA3⁻, SSEA4⁻, ABC-p⁺, KDR⁻ (VEGFR2⁻), HLA-A,B,C⁺, HLA-DP,DQ,DR⁻, HLA-G, or Programmed Death-1 Ligand (PDL1)⁺, or any combination thereof. In another specific embodiment, the CD34⁻, CD10⁺, CD105⁺ cells are additionally CD13⁺, CD29⁺, CD33⁺, CD38⁻, CD44⁺, CD45⁻, CD54/ICAM⁺, CD62E⁻, CD62L⁻, CD62P⁻, SH3⁺ (CD73⁺), SH4⁺ (CD73⁺), CD80⁻, CD86⁻, CD90⁺, SH2+(CD105⁺), CD106/VCAM⁺, CD117⁻, CD144/VE-cadherin^{low}, CD184/CXCR4⁻, CD200⁺, CD133, OCT-4⁺, SSEA3⁻, SSEA4⁻, ABC-p⁺, KDR⁻ (VEGFR2), HLA-A,B,C⁺, HLA-DP,DQ,DR⁻, HLA-G, and Programmed Death-1 Ligand (PDL1)⁺.

In certain embodiments, the isolated placental adherent cells useful in the methods and compositions described herein are one or more, or all, of CD10⁺, CD29⁺, CD34⁻, CD38⁻, CD44⁺, CD45⁻, CD54⁺, CD90⁺, SH2⁺, SH3⁺, SH4⁺, SSEA3⁻, SSEA4, OCT-4⁺, and ABC-p⁺, wherein said isolated placental adherent cells are obtained by physical and/or enzymatic disruption of placental tissue. In a specific embodiment, the isolated placental adherent cells are OCT-4⁺ and ABC-p⁺. In another specific embodiment, the isolated placental adherent cells are OCT-4⁺ and CD34, wherein said isolated placental adherent cells have at least one of the following characteristics: CD10⁺, CD29⁺, CD44⁺, CD45⁻, CD54⁺, CD90⁺, SH3⁺, SH4⁺, SSEA4⁻, and SSEA4⁻. In another specific embodiment, the isolated placental adherent cells are OCT-4⁺, CD34⁻, CD10⁺, CD29⁺, CD44⁺, CD45⁻, CD54⁺, CD90⁺, SH3⁺, SH4⁺, SSEA3⁻, and SSEA4⁻. In another embodiment, the isolated placental adherent cells are OCT-4⁺, CD34⁻, SSEA4⁻, and SSEA4⁻. In another specific embodiment, the isolated placental adherent cells are OCT-4⁺ and CD34⁻, and is either SH2⁺ or SH3⁺. In another specific embodiment, the isolated placental adherent cells are OCT-4⁺, CD34⁻, SH2⁺, and SH3⁺. In another specific embodiment, the isolated placental adherent cells are OCT-4⁺, CD34⁻, SSEA3⁻, and SSEA4⁻, and are either SH2⁺ or SH3⁺. In another specific embodiment, the isolated placental adherent cells are OCT-4⁺ and CD34⁻, and either SH2⁺ or SH3⁺, and is at least one of CD10⁺, CD29⁺, CD44⁺, CD45⁻, CD54⁺, CD90⁺, SSEA3⁻, or SSEA4⁻. In another specific embodiment, the isolated placental adherent cells are OCT-4⁺, CD34⁻, CD10⁺, CD29⁺, CD44⁺, CD45⁻, CD54⁺, CD90⁺, SSEA3⁻, and SSEA4⁻, and either SH2⁺ or SH3⁺.

In another embodiment, the isolated placental adherent cells useful in the methods and compositions disclosed herein are SH2⁺, SH3⁺, SH4⁺ and OCT-4⁺. In another specific embodiment, the isolated placental adherent cells are CD10⁺, CD29⁺, CD44⁺, CD54⁺, CD90⁺, CD34⁻, CD45⁻, SSEA4⁻, or SSEA4⁻. In another embodiment, the isolated placental adherent cells are SH2⁺, SH3⁺, SH4⁺, SSEA4⁻ and SSEA4⁻. In another specific embodiment, the isolated placental adherent cells are SH2⁺, SH3⁺, SH4⁺, SSEA3⁻ and SSEA4⁻, CD10⁺, CD29⁺, CD44⁺, CD54⁺, CD90⁺, OCT-4⁺, CD34⁻ or CD45⁻.

In another embodiment, the isolated placental adherent cells useful in the methods and compositions disclosed herein are CD10⁺, CD29⁺, CD34⁻, CD44⁺, CD45⁻, CD54⁺, CD90⁺, SH2⁺, SH3⁺, and SH4⁺; wherein said isolated placental adherent cells are additionally one or more of OCT-4⁺, SSEA4⁻ or SSEA4⁻.

In certain embodiments, isolated placental adherent cells useful in the methods and compositions disclosed herein are CD200⁺ or HLA-G⁻. In a specific embodiment, the isolated placental adherent cells are CD200⁺ and HLA-G⁻ In another specific embodiment, the isolated placental adherent cells are additionally CD73⁺ and CD105⁺. In another specific embodiment, the isolated placental adherent cells are additionally CD34⁻, CD38⁻ or CD45⁻. In another specific embodiment, the isolated placental adherent cells are additionally CD34⁻, CD38⁻ and CD45⁻. In another specific embodiment, said stem cells are CD34⁻, CD38⁻, CD45⁻, CD73⁺ and CD105⁺. In another specific embodiment, said isolated CD200⁺ or HLA-G⁻ placental adherent cells facilitate the formation of embryoid-like bodies in a population of placental adherent cells comprising the isolated placental adherent cells, under conditions that allow the formation of embryoid-like bodies. In another specific embodiment, the isolated placental adherent cells are isolated away from placental adherent cells that are not stem or multipotent cells. In another specific embodiment, said isolated placental adherent cells are isolated away from placental adherent cells that do not display these markers.

In another embodiment, a cell population useful in the methods and compositions described herein is a population of cells comprising, e.g., that is enriched for, CD200⁺, HLA-G⁻ placental adherent cells. In a specific embodiment, said population is a population of placental adherent cells. In various embodiments, at least about 10%, at least about 20%, at least about 30%, at least about 40%, at least about 50%, or at least about 60% of cells in said cell population are isolated CD200⁺, HLA-G⁻ placental adherent cells. Preferably, at least about 70% of cells in said cell population are isolated CD200⁺, HLA-G⁻ placental adherent cells. More preferably, at least about 90%, 95%, or 99% of said cells are isolated CD200⁺, HLA-G⁻ placental adherent cells. In a specific embodiment of the cell populations, said isolated CD200⁺, HLA-G⁻ placental adherent cells are also CD73⁺ and CD105⁺. In another specific embodiment, said isolated CD200⁺, HLA-G⁻ placental adherent cells are also CD34⁻, CD38⁻ or CD45⁻. In another specific embodiment, said isolated CD200⁺, HLA-G⁻ placental adherent cells are also CD34⁻, CD38⁻, CD45⁻, CD73⁺ and CD105⁺. In another embodiment, said cell population produces one or more embryoid-like bodies when cultured under conditions that allow the formation of embryoid-like bodies. In another specific embodiment, said cell population is isolated away from placental adherent cells that are not stem cells. In another specific embodiment, said isolated CD200⁺, HLA-G⁻ placental adherent cells are isolated away from placental adherent cells that do not display these markers.

In another embodiment, the isolated placental adherent cells useful in the methods and compositions described herein are CD73⁺, CD105⁺, and CD200⁺. In another specific embodiment, the isolated placental adherent cells are HLA-G⁻. In another specific embodiment, the isolated placental adherent cells are CD34⁻, CD38⁻ or CD45⁻. In another specific embodiment, the isolated placental adherent cells are CD34⁻, CD38⁻ and CD45⁻. In another specific embodiment, the isolated placental adherent cells are CD34⁻, CD38⁻, CD45⁻, and HLA-G⁻. In another specific embodiment, the isolated CD73⁺, CD105⁺, and CD200⁺ placental adherent cells facilitate the formation of one or more embryoid-like bodies in a population of placental adherent cells comprising the isolated placental adherent cells, when the population is cultured under conditions that allow the formation of embryoid-like bodies. In another specific embodiment, the isolated placental adherent cells are isolated away from placental adherent cells that are not the isolated placental adherent cells. In another specific embodiment, the isolated placental adherent cells are isolated away from placental adherent cells that do not display these markers.

In another embodiment, a cell population useful in the methods and compositions described herein is a population of cells comprising, e.g., that is enriched for, isolated CD73⁺, CD105⁺, CD200⁺ placental adherent cells. In various embodiments, at least about 10%, at least about 20%, at least about 30%, at least about 40%, at least about 50%, or at least about 60% of cells in said cell population are isolated CD73⁺, CD105⁺, CD200⁺ placental adherent cells. In another embodiment, at least about 70% of said cells in said population of cells are isolated CD73⁺, CD105⁺, CD200⁺ placental adherent cells. In another embodiment, at least about 90%, 95% or 99% of cells in said population of cells are isolated CD73⁺, CD105⁺, CD200⁺ placental adherent cells. In a specific embodiment of said populations, the isolated placental adherent cells are HLA-G⁻. In another specific embodiment, the isolated placental adherent cells are additionally CD34⁻, CD38⁻ or CD45⁻. In another specific embodiment, the isolated placental adherent cells are additionally CD34⁻, CD38⁻ and CD45⁻. In another specific embodiment, the isolated placental adherent cells are additionally CD34⁻, CD38⁻, CD45⁻, and HLA-G⁻. In another specific embodiment, said population of cells produces one or more embryoid-like bodies when cultured under conditions that allow the formation of embryoid-like bodies. In another specific embodiment, said population of placental adherent cells is isolated away from placental adherent cells that are not stem cells. In another specific embodiment, said population of placental adherent cells is isolated away from placental adherent cells that do not display these characteristics.

In certain other embodiments, the isolated placental adherent cells are one or more of CD10⁺, CD29⁺, CD34⁻, CD38⁻, CD44⁺, CD45⁻, CD54⁺, CD90⁺, SH2⁺, SH3⁺, SH4⁺, SSEA3⁻, SSEA4⁻, OCT-4⁻, HLA-G⁻ or ABC-p⁺. In a specific embodiment, the isolated placental adherent cells are CD10⁺, CD29⁺, CD34⁻, CD38⁻, CD44⁺, CD45⁻, CD54⁺, CD90⁺, SH2⁺, SH3⁺, SH4⁺, SSEA3⁻, SSEA4⁻, and OCT-4⁺. In another specific embodiment, the isolated placental adherent cells are CD10⁺, CD29⁺, CD34⁻, CD38⁻, CD45⁻, CD54⁺, SH2⁺, SH3⁺, and SH4⁺. In another specific embodiment, the isolated placental adherent cells are CD10⁺, CD29⁺, CD34⁻, CD38⁻, CD45⁻, CD54⁺, SH2⁺, SH3⁺, SH4⁺ and OCT-4⁺. In another specific embodiment, the isolated placental adherent cells are CD10⁺, CD29⁺, CD34⁻, CD38⁻, CD44⁺, CD45⁻, CD54⁺, CD90⁺, HLA-G⁻, SH2⁺, SH3⁺, SH4⁺. In another specific embodiment, the isolated placental adherent cells are OCT-4⁺ and ABC-p⁺. In another specific embodiment, the isolated placental adherent cells are SH2⁺, SH3⁺, SH4⁺ and OCT-4⁺. In another embodiment, the isolated placental adherent cells are OCT-4⁺, CD34⁻, SSEA3⁻, and SSEA4⁻. In a specific embodiment, said isolated OCT-4⁺, CD34⁻, SSEA3⁻, and SSEA4⁻ placental adherent cells are additionally CD10⁺, CD29⁺, CD34⁻, CD44⁺, CD45⁻, CD54⁺, CD90⁺, SH2⁺, SH3⁺, and SH4⁺. In another embodiment, the isolated placental adherent cells are OCT-4⁺ and CD34⁻, and either SH3⁺ or SH4⁺. In another embodiment, the isolated placental adherent cells are CD34⁻ and either CD10⁺, CD29⁺, CD44⁺, CD54⁺, CD90⁺, or OCT-4⁺.

In another embodiment, the isolated placental adherent cells useful in the methods and compositions described herein are CD200⁺ and OCT-4⁺. In a specific embodiment, the isolated placental adherent cells are CD73⁺ and CD105⁺. In another specific embodiment, said isolated placental adherent cells are HLA-G⁻. In another specific embodiment, said isolated CD200⁺, OCT-4⁺ placental adherent cells are CD34⁻, CD38⁻ or CD45⁻. In another specific embodiment, said isolated CD200⁺, OCT-4⁺ placental adherent cells are CD34⁻, CD38⁻ and CD45⁻. In another specific embodiment, said isolated CD200⁺, OCT-4⁺ placental adherent cells are CD34⁻, CD38⁻, CD45⁻, CD73⁺, CD105⁺ and HLA-G⁻. In another specific embodiment, the isolated CD200⁺, OCT-4⁺ placental adherent cells facilitate the production of one or more embryoid-like bodies by a population of placental adherent cells that comprises the isolated cells, when the population is cultured under conditions that allow the formation of embryoid-like bodies. In another specific embodiment, said isolated CD200⁺, OCT-4⁺ placental adherent cells are isolated away from placental adherent cells that are not stem cells. In another specific embodiment, said isolated CD200⁺, OCT-4⁺ placental adherent cells are isolated away from placental adherent cells that do not display these characteristics.

In another embodiment, a cell population useful in the methods and compositions described herein is a population of cells comprising, e.g., that is enriched for, CD200⁺, OCT-4⁺ placental adherent cells. In various embodiments, at least about 10%, at least about 20%, at least about 30%, at least about 40%, at least about 50%, or at least about 60% of cells in said cell population are isolated CD200⁺, OCT-4⁺ placental adherent cells. In another embodiment, at least about 70% of said cells are said isolated CD200⁺, OCT-4⁺ placental adherent cells. In another embodiment, at least about 80%, 90%, 95%, or 99% of cells in said cell population are said isolated CD200⁺, OCT-4⁺ placental adherent cells. In a specific embodiment of the isolated populations, said isolated CD200⁺, OCT-4⁺ placental adherent cells are additionally CD73⁺ and CD105⁺. In another specific embodiment, said isolated CD200⁺, OCT-4⁺ placental adherent cells are additionally HLA-G⁻. In another specific embodiment, said isolated CD200⁺, OCT-4⁺ placental adherent cells are additionally CD34⁻, CD38⁻ and CD45⁻. In another specific embodiment, said isolated CD200⁺, OCT-4⁺ placental adherent cells are additionally CD34⁻, CD38⁻, CD45⁻, CD73⁺, CD105⁺ and HLA-G⁻. In another specific embodiment, the cell population produces one or more embryoid-like bodies when cultured under conditions that allow the formation of embryoid-like bodies. In another specific embodiment, said cell population is isolated away from placental adherent cells that are not isolated CD200⁺, OCT-4⁺ placental adherent cells. In another specific embodiment, said cell population is isolated away from placental adherent cells that do not display these markers.

In another embodiment, the isolated placental adherent cells useful in the methods and compositions described herein are CD73⁺, CD105⁺ and HLA-G⁻. In another specific embodiment, the isolated CD73⁺, CD105⁺ and HLA-G⁻ placental adherent cells are additionally CD34⁻, CD38⁻ or CD45⁻. In another specific embodiment, the isolated CD73, CD105⁺, HLA-G⁻ placental adherent cells are additionally CD34⁻, CD38⁻ and CD45⁻. In another specific embodiment, the isolated CD73⁺, CD105⁺, HLA-G⁻ placental adherent cells are additionally OCT-4⁺. In another specific embodiment, the isolated CD73⁺, CD105⁺, HLA-G⁻ placental adherent cells are additionally CD200⁺. In another specific embodiment, the isolated CD73⁺, CD105⁺, HLA-G⁻ placental adherent cells are additionally CD34⁻, CD38⁻, CD45⁻, OCT-4⁺ and CD200⁺. In another specific embodiment, the isolated CD73⁺, CD105⁺, HLA-G⁻ placental adherent cells facilitate the formation of embryoid-like bodies in a population of placental adherent cells comprising said cells, when the population is cultured under conditions that allow the formation of embryoid-like bodies. In another specific embodiment, said the isolated CD73⁺, CD105⁺, HLA-G⁻ placental adherent cells are isolated away from placental adherent cells that are not the isolated CD73⁺, CD105⁺, HLA-G placental adherent cells. In another specific embodiment, said the isolated CD73⁺, CD105⁺, HLA-G⁻ placental adherent cells are isolated away from placental adherent cells that do not display these markers.

In another embodiment, a cell population useful in the methods and compositions described herein is a population of cells comprising, e.g., that is enriched for, isolated CD73⁺, CD105⁺ and HLA-G⁻ placental adherent cells. In various embodiments, at least about 10%, at least about 20%, at least about 30%, at least about 40%, at least about 50%, or at least about 60% of cells in said population of cells are isolated CD73⁺, CD105⁺, HLA-G⁻ placental adherent cells. In another embodiment, at least about 70% of cells in said population of cells are isolated CD73⁺, CD105⁺, HLA-G⁻ placental adherent cells. In another embodiment, at least about 90%, 95% or 99% of cells in said population of cells are isolated CD73⁺, CD105⁺, HLA-G⁻ placental adherent cells. In a specific embodiment of the above populations, said isolated CD73⁺, CD105⁺, HLA-G⁻ placental adherent cells are additionally CD34⁻, CD38⁻ or CD45⁻. In another specific embodiment, said isolated CD73⁺, CD105⁺, HLA-G⁻ placental adherent cells are additionally CD34⁻, CD38⁻ and CD45⁻. In another specific embodiment, said isolated CD73⁺, CD105⁺, HLA-G⁻ placental adherent cells are additionally OCT-4⁺. In another specific embodiment, said isolated CD73⁺, CD105⁺, HLA-G⁻ placental adherent cells are additionally CD200⁺. In another specific embodiment, said isolated CD73⁺, CD105⁺, HLA-G⁻ placental adherent cells are additionally CD34⁻, CD38⁻, CD45⁻, OCT-4⁺ and CD200⁺. In another specific embodiment, said cell population is isolated away from placental adherent cells that are not CD73⁺, CD105⁺, HLA-G⁻ placental adherent cells. In another specific embodiment, said cell population is isolated away from placental adherent cells that do not display these markers.

In another embodiment, the isolated placental adherent cells useful in the methods and compositions described herein are CD73⁺ and CD105⁺ and facilitate the formation of one or more embryoid-like bodies in a population of isolated placental adherent cells comprising said CD73⁺, CD105⁺ cells when said population is cultured under conditions that allow formation of embryoid-like bodies. In another specific embodiment, said isolated CD73⁺, CD105⁺ placental adherent cells are additionally CD34⁻, CD38⁻ or CD45⁻. In another specific embodiment, said isolated CD73⁺, CD105⁺ placental adherent cells are additionally CD34⁻, CD38⁻ and CD45⁻. In another specific embodiment, said isolated CD73⁺, CD105⁺ placental adherent cells are additionally OCT-4⁺. In another specific embodiment, said isolated CD73⁺, CD105⁺ placental adherent cells are additionally OCT-4⁺, CD34⁻, CD38⁻ and CD45⁻. In another specific embodiment, said isolated CD73⁺, CD105⁺ placental adherent cells are isolated away from placental adherent cells that are not said cells. In another specific embodiment, said isolated CD73⁺, CD105⁺ placental adherent cells are isolated away from placental adherent cells that do not display these characteristics.

In another embodiment, a cell population useful in the methods and compositions described herein is a population of cells comprising, e.g., that is enriched for, isolated placental adherent cells that are CD73⁺, CD105⁺ and facilitate the formation of one or more embryoid-like bodies in a population of isolated placental adherent cells comprising said cells when said population is cultured under conditions that allow formation of embryoid-like bodies. In various embodiments, at least about 10%, at least about 20%, at least about 30%, at least about 40%, at least about 50%, or at least about 60% of cells in said population of cells are said isolated CD73⁺, CD105⁺ placental adherent cells. In another embodiment, at least about 70% of cells in said population of cells are said isolated CD73⁺, CD105⁺ placental adherent cells. In another embodiment, at least about 90%, 95% or 99% of cells in said population of cells are said isolated CD73⁺, CD105⁺ placental adherent cells. In a specific embodiment of the above populations, said isolated CD73⁺, CD105⁺ placental adherent cells are additionally CD34⁻, CD38⁻ or CD45⁻. In another specific embodiment, said isolated CD73⁺, CD105⁺ placental adherent cells are additionally CD34⁻, CD38⁻ and CD45⁻. In another specific embodiment, said isolated CD73⁺, CD105⁺ placental adherent cells are additionally OCT-4⁺. In another specific embodiment, said isolated CD73⁺, CD105⁺ placental adherent cells are additionally CD200⁺. In another specific embodiment, said isolated CD73⁺, CD105⁺ placental adherent cells are additionally CD34⁻, CD38⁻, CD45⁻, OCT-4⁺ and CD200⁺. In another specific embodiment, said cell population is isolated away from placental adherent cells that are not said isolated CD73⁺, CD105⁺ placental adherent cells. In another specific embodiment, said cell population is isolated away from placental adherent cells that do not display these markers.

In another embodiment, the isolated placental adherent cells useful in the methods and compositions described herein are OCT-4⁺ and facilitate formation of one or more embryoid-like bodies in a population of isolated placental adherent cells comprising said cells when cultured under conditions that allow formation of embryoid-like bodies. In a specific embodiment, said isolated OCT-4⁺ placental adherent cells are additionally CD73⁺ and CD105⁺. In another specific embodiment, said isolated OCT-4⁺ placental adherent cells are additionally CD34⁻, CD38⁻, or CD45⁻. In another specific embodiment, said isolated OCT-4⁺ placental adherent cells are additionally CD200⁺. In another specific embodiment, said isolated OCT-4⁺ placental adherent cells are additionally CD73⁺, CD105⁺, CD200⁺, CD34⁻, CD38⁻, and CD45⁻. In another specific embodiment, said isolated OCT-4⁺ placental adherent cells are isolated away from placental adherent cells that are not OCT-4⁺ placental adherent cells. In another specific embodiment, said isolated OCT-4⁺ placental adherent cells are isolated away from placental adherent cells that do not display these characteristics.

In another embodiment, a cell population useful in the methods and compositions described herein is a population of cells comprising, e.g., that is enriched for, isolated placental adherent cells that are OCT-4⁺ and facilitate the formation of one or more embryoid-like bodies in a population of isolated placental adherent cells comprising said cells when said population is cultured under conditions that allow formation of embryoid-like bodies. In various embodiments, at least about 10%, at least about 20%, at least about 30%, at least about 40%, at least about 50%, or at least about 60% of cells in said population of cells are said isolated OCT-4⁺ placental adherent cells. In another embodiment, at least about 70% of cells in said population of cells are said isolated OCT-4⁺ placental adherent cells. In another embodiment, at least about 80%, 90%, 95% or 99% of cells in said population of cells are said isolated OCT-4⁺ placental adherent cells. In a specific embodiment of the above populations, said isolated OCT-4⁺ placental adherent cells are additionally CD34⁻, CD38⁻ or CD45⁻. In another specific embodiment, said isolated OCT-4⁺ placental adherent cells are additionally CD34⁻, CD38⁻ and CD45⁻. In another specific embodiment, said isolated OCT-4⁺ placental adherent cells are additionally CD73⁺ and CD105⁺. In another specific embodiment, said isolated OCT-4⁺ placental adherent cells are additionally CD200⁺. In another specific embodiment, said isolated OCT-4⁺ placental adherent cells are additionally CD73⁺, CD105⁺, CD200⁺, CD34⁻, CD38⁻, and CD45⁻. In another specific embodiment, said cell population is isolated away from placental adherent cells that are not said cells. In another specific embodiment, said cell population is isolated away from placental adherent cells that do not display these markers.

In another embodiment, the isolated placental adherent cells useful in the methods and compositions described herein are isolated HLA-A,B,C⁺, CD45⁻, CD133⁻ and CD34⁻ placental adherent cells. In another embodiment, a cell population useful in the methods and compositions described herein is a population of cells comprising isolated placental adherent cells, wherein at least about 70%, at least about 80%, at least about 90%, at least about 95% or at least about 99% of cells in said isolated population of cells are isolated HLA-A,B,C⁺, CD45⁻, CD133⁻ and CD34⁻ placental adherent cells. In a specific embodiment, said isolated placental cell or population of isolated placental adherent cells is isolated away from placental adherent cells that are not HLA-A,B,C⁺, CD45⁻, CD133⁻ and CD34⁻ placental adherent cells. In another specific embodiment, said isolated placental adherent cells are non-maternal in origin. In another specific embodiment, said isolated population of placental adherent cells are substantially free of maternal components; e.g., at least about 40%, 45%, 5-0%, 55%, 60%, 65%, 70%, 75%, 90%, 85%, 90%, 95%, 98% or 99% of said cells in said isolated population of placental adherent cells are non-maternal in origin.

In another embodiment, the isolated placental adherent cells useful in the methods and compositions described herein are isolated CD10⁺, CD13⁺, CD33⁺, CD45⁻, CD117 and CD133⁻ placental adherent cells. In another embodiment, a cell population useful in the methods and compositions described herein is a population of cells comprising isolated placental adherent cells, wherein at least about 70%, at least about 80%, at least about 90%, at least about 95% or at least about 99% of cells in said population of cells are isolated CD10⁺, CD13⁺, CD33⁺, CD45⁻, CD117⁻ and CD133⁻ placental adherent cells. In a specific embodiment, said isolated placental adherent cells or population of isolated placental adherent cells is isolated away from placental adherent cells that are not said isolated placental adherent cells. In another specific embodiment, said isolated CD10⁺, CD13⁺, CD33⁺, CD45⁻, CD117⁻ and CD133⁻ placental adherent cells are non-maternal in origin, i.e., have the fetal genotype. In another specific embodiment, at least about 40%, 45%, 50%, 55%, 60%, 65%, 70%, 75%, 90%, 85%, 90%, 95%, 98% or 99% of said cells in said isolated population of placental adherent cells, are non-maternal in origin. In another specific embodiment, said isolated placental adherent cells or population of isolated placental adherent cells are isolated away from placental adherent cells that do not display these characteristics.

In another embodiment, the isolated placental adherent cells useful in the methods and compositions described herein are isolated CD10⁻, CD33⁻, CD44⁺, CD45⁻, and CD117-placental adherent cells. In another embodiment, a cell population useful for the in the methods and compositions described herein is a population of cells comprising, e.g., enriched for, isolated placental adherent cells, wherein at least about 70%, at least about 80%, at least about 90%, at least about 95% or at least about 99% of cells in said population of cells are isolated CD10⁻, CD33⁻, CD44⁺, CD45⁻, and CD117⁻ placental adherent cells. In a specific embodiment, said isolated placental cell or population of isolated placental adherent cells is isolated away from placental adherent cells that are not said cells. In another specific embodiment, said isolated placental adherent cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin. In another specific embodiment, said isolated placental cell or population of isolated placental adherent cells is isolated away from placental adherent cells that do not display these markers.

In another embodiment, the isolated placental adherent cells useful in the methods and compositions described herein are isolated CD10⁻, CD13⁻, CD33⁻, CD45⁻, and CD117-placental adherent cells. In another embodiment, a cell population useful for in the methods and compositions described herein is a population of cells comprising, e.g., enriched for, isolated CD10⁻, CD13⁻, CD33⁻, CD45⁻, and CD117⁻ placental adherent cells, wherein at least about 70%, at least about 80%, at least about 90%, at least about 95% or at least about 99% of cells in said population are CD10⁻, CD13⁻, CD33⁻, CD45⁻, and CD117⁻ placental adherent cells. In a specific embodiment, said isolated placental adherent cells or population of isolated placental adherent cells are isolated away from placental adherent cells that are not said cells. In another specific embodiment, said isolated placental adherent cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin. In another specific embodiment, said isolated placental adherent cells or population of isolated placental adherent cells is isolated away from placental adherent cells that do not display these characteristics.

In another embodiment, the isolated placental adherent cells useful in the methods and compositions described herein are HLA A,B,C⁺, CD45⁻, CD34⁻, and CD133⁻, and are additionally CD10⁺, CD13⁺, CD38⁺, CD44⁺, CD90⁺, CD105⁺, CD200⁺ and/or HLA-G⁻, and/or negative for CD117. In another embodiment, a cell population useful in the methods described herein is a population of cells comprising isolated placental adherent cells, wherein at least about 20%, 25%, 30%, 35%, 40%, 45%, 50%, 55%, 60%, 65%, 70%, 75%, 80%, 85%, 90%, 95%, 98% or about 99% of the cells in said population are isolated placental adherent cells that are HLA A,B,C⁻, CD45⁻, CD34⁻, CD133⁻, and that are additionally positive for CD10, CD13, CD38, CD44, CD90, CD105, CD200, and/or negative for CD117 and/or HLA-G. In a specific embodiment, said isolated placental adherent cells or population of isolated placental adherent cells are isolated away from placental adherent cells that are not said cells. In another specific embodiment, said isolated placental adherent cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin. In another specific embodiment, said isolated placental adherent cells or population of isolated placental adherent cells are isolated away from placental adherent cells that do not display these markers.

In another embodiment, the isolated placental adherent cells useful in the methods and compositions described herein are isolated placental adherent cells that are CD200⁺ and CD10⁺, as determinable by antibody binding, and CD117⁻, as determinable by both antibody binding and RT-PCR. In another embodiment, the isolated placental adherent cells useful in the methods and compositions described herein are isolated placental adherent cells, e.g., placental stem cells or placental multipotent cells, that are CD10⁺, CD29⁻, CD54⁺, CD200⁺, HLA-G⁻, MHC class I⁺ and β-2-microglobulin⁺. In another embodiment, isolated placental adherent cells useful in the methods and compositions described herein are placental adherent cells wherein the expression of at least one cellular marker is at least two-fold higher than for a mesenchymal stem cell (e.g., a bone marrow-derived mesenchymal stem cell). In another specific embodiment, said isolated placental adherent cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin.

In another embodiment, the isolated placental adherent cells useful in the methods and compositions described herein are isolated placental adherent cells, e.g., placental stem cells or placental multipotent cells, that are one or more of CD10⁺, CD29⁺, CD44⁺, CD45⁻, CD54/ICAM⁺, CD62E⁻, CD62L⁻, CD62P⁻, CD80⁻, CD86⁻, CD103⁻, CD104⁻, CD105⁺, CD106/VCAM⁺, CD144/VE-cadherin^{low}, CD184/CXCR4⁻, 02-microglobulin^{low}, MHC-IV, HLA-G^{low}, and/or PDL1^{low}. In a specific embodiment, the isolated placental adherent cells are at least CD29⁺ and CD54⁺. In another specific embodiment, the isolated placental adherent cells are at least CD44⁺ and CD106⁺. In another specific embodiment, the isolated placental adherent cells are at least CD29⁺.

In another embodiment, a cell population useful in the methods and compositions described herein comprises isolated placental adherent cells, and at least 50%, 60%, 70%, 80%, 90%, 95%, 98% or 99% of the cells in said cell population are isolated placental adherent cells that are one or more of CD10⁺, CD29⁺, CD44⁺, CD45⁻, CD54/ICAM⁺, CD62-E⁻, CD62-L⁻, CD62-P⁻, CD80⁻, CD86⁻, CD103⁻, CD104⁻, CD105⁺, CD106/VCAM⁺, CD144/VE-cadherin^{dim}, CD184/CXCR4⁻, 02-microglobulin^{dim}, HLA-I^{dim}, HLA-II⁻, HLA-G^{dim}, and/or PDL1^{dim}. In another specific embodiment, at least 50%, 60%, 70%, 80%, 90%, 95%, 98% or 99% of cells in said cell population are CD10⁺, CD29⁺, CD44⁺, CD45⁻, CD54/ICAM⁺, CD62-E⁻, CD62-L⁻, CD62-P⁻, CD80⁻, CD86⁻, CD103⁻, CD104⁻, CD105⁺, CD106/VCAM⁺, CD144/VE-cadherin^{dim}, CD184/CXCR4⁻, 02-microglobulin^{dim}, HLA-I^{dim}, MHC-II⁻, HLA-G^{dim}, and PDL1^{dim}.

In another embodiment, the isolated placental adherent cells useful in the methods and compositions described herein are isolated placental adherent cells that are one or more, or all, of CD10⁺, CD29⁺, CD34⁻, CD38⁻, CD44⁺, CD45⁻, CD54⁺, CD90⁺, SH2⁺, SH3⁺, SH4⁺, SSEA3⁻, SSEA4⁻, OCT-4⁺, and ABC-p⁺, where ABC-p is a placenta-specific ABC transporter protein (also known as breast cancer resistance protein (BCRP) and as mitoxantrone resistance protein (MXR)), wherein said isolated placental adherent cells are obtained by perfusion of a mammalian, e.g., human, placenta that has been drained of cord blood and perfused to remove residual blood.

In another specific embodiment of any of the above characteristics, expression of the cellular marker (e.g., cluster of differentiation or immunogenic marker) is determinable by flow cytometry; in another specific embodiment, expression of the marker is determinable by RT-PCR.

Gene profiling confirms that isolated placental adherent cells, and populations of isolated placental adherent cells, are distinguishable from other cells, e.g., mesenchymal stem cells, e.g., bone marrow-derived mesenchymal stem cells. The isolated placental adherent cells described herein can be distinguished from, e.g., mesenchymal stem cells on the basis of the expression of one or more genes, the expression of which is significantly higher in the isolated placental adherent cells, or in certain isolated umbilical cord stem cells, in comparison to bone marrow-derived mesenchymal stem cells. In particular, the isolated placental adherent cells, useful in the methods of treatment provided herein, can be distinguished from mesenchymal stem cells on the basis of the expression of one or more genes, the expression of which is significantly higher (that is, at least twofold higher) in the isolated placental adherent cells than in an equivalent number of bone marrow-derived mesenchymal stem cells, wherein the one or more genes are ACTG2, ADARB1, AMIGO2, ARTS-1, B4GALT6, BCHE, C11orf9, CD200, COL4A1, COL4A2, CPA4, DMD, DSC3, DSG2, ELOVL2, F2RL1, FLJ10781, GATA6, GPR126, GPRC5B, HLA-G, ICAM1, IER3, IGFBP7, IL1A, IL6, IL18, KRT18, KRT8, LIPG, LRAP, MATN2, MEST, NFE2L3, NUAK1, PCDH7, PDLIM3, PKP2, RTN1, SERPINB9, ST3GAL6, ST6GALNAC5, SLC12A8, TCF21, TGFB2, VTN, ZC3H12A, or a combination of any of the foregoing, when the cells are grown under equivalent conditions. See, e.g., U.S. Patent Application Publication No. 2007/0275362, the disclosure of which is incorporated herein by reference in its entirety. In certain specific embodiments, said expression of said one ore more genes is determined, e.g., by RT-PCR or microarray analysis, e.g, using a U133-A microarray (Affymetrix). In another specific embodiment, said isolated placental adherent cells express said one or more genes when cultured for a number of population doublings, e.g., anywhere from about 3 to about 35 population doublings, in a medium comprising DMEM-LG (e.g., from Gibco); 2% fetal calf serum (e.g., from Hyclone Labs.); 1× insulin-transferrin-selenium (ITS); 1× linoleic acid-bovine serum albumin (LA-BSA); 10⁻9 M dexamethasone (e.g., from Sigma); 10⁻4 M ascorbic acid 2-phosphate (e.g., from Sigma); epidermal growth factor 10 ng/mL (e.g., from R&D Systems); and platelet-derived growth factor (PDGF-BB) 10 ng/mL (e.g., from R&D Systems). In another specific embodiment, the isolated placental cell-specific or isolated umbilical cord cell-specific gene is CD200.

Specific sequences for these genes can be found in GenBank at accession nos. NM_001615 (ACTG2), BC065545 (ADARB1), (NM_181847 (AMIGO2), AY358590 (ARTS-1), BC074884 (B4GALT6), BC008396 (BCHE), BC020196 (C11orf9), BC031103 (CD200), NM_001845 (COL4A1), NM_001846 (COL4A2), BC052289 (CPA4), BC094758 (DMD), AF293359 (DSC3), NM_001943 (DSG2), AF338241 (ELOVL2), AY336105 (F2RL1), NM_018215 (FLJ10781), AY416799 (GATA6), BC075798 (GPR126), NM_016235 (GPRC5B), AF340038 (ICAM1), BC000844 (IER3), BC066339 (IGFBP7), BC013142 (IL1A), BT019749 (IL6), BC007461 (IL18), (BC072017) KRT18, BC075839 (KRT8), BC060825 (LIPG), BC065240 (LRAP), BC010444 (MATN2), BC011908 (MEST), BC068455 (NFE2L3), NM_014840 (NUAK1), AB006755 (PCDH7), NM_014476 (PDLIM3), BC126199 (PKP-2), BC090862 (RTN1), BC002538 (SERPINB9), BC023312 (ST3GAL6), BC001201 (ST6GALNAC5), BC126160 or BC065328 (SLC12A8), BC025697 (TCF21), BC096235 (TGFB2), BC005046 (VTN), and BC005001 (ZC3H12A) as of March 2008.

In certain specific embodiments, said isolated placental adherent cells express each of ACTG2, ADARB1, AMIGO2, ARTS-1, B4GALT6, BCHE, C11orf9, CD200, COL4A1, COL4A2, CPA4, DMD, DSC3, DSG2, ELOVL2, F2RL1, F1110781, GATA6, GPR126, GPRC5B, HLA-G, ICAM1, IER3, IGFBP7, IL1A, IL6, IL18, KRT18, KRT8, LIPG, LRAP, MATN2, MEST, NFE2L3, NUAK1, PCDH7, PDLIM3, PKP2, RTN1, SERPINB9, ST3GAL6, ST6GALNAC5, SLC12A8, TCF21, TGFB2, VTN, and ZC3H12A at a detectably higher level than an equivalent number of bone marrow-derived mesenchymal stem cells, when the cells are grown under equivalent conditions.

In specific embodiments, the placental adherent cells express CD200 and ARTS1 (aminopeptidase regulator of type 1 tumor necrosis factor); ARTS-1 and LRAP (leukocyte-derived arginine aminopeptidase); IL6 (interleukin-6) and TGFB2 (transforming growth factor, beta 2); IL6 and KRT18 (keratin 18); IER3 (immediate early response 3), MEST (mesoderm specific transcript homolog) and TGFB2; CD200 and IER3; CD200 and IL6; CD200 and KRT18; CD200 and LRAP; CD200 and MEST; CD200 and NFE2L3 (nuclear factor (erythroid-derived 2)-like 3); or CD200 and TGFB2 at a detectably higher level than an equivalent number of bone marrow-derived mesenchymal stem cells (BM-MSCs) wherein said bone marrow-derived mesenchymal stem cells have undergone a number of passages in culture equivalent to the number of passages said isolated placental adherent cells have undergone. In other specific embodiments, the placental adherent cells express ARTS-1, CD200, IL6 and LRAP; ARTS-1, IL6, TGFB2, IER3, KRT18 and MEST; CD200, IER3, IL6, KRT18, LRAP, MEST, NFE2L3, and TGFB2; ARTS-1, CD200, IER3, IL6, KRT18, LRAP, MEST, NFE2L3, and TGFB2; or IER3, MEST and TGFB2 at a detectably higher level than an equivalent number of bone marrow-derived mesenchymal stem cells BM-MSCs, wherein said bone marrow-derived mesenchymal stem cells have undergone a number of passages in culture equivalent to the number of passages said isolated placental adherent cells have undergone.

Expression of the above-referenced genes can be assessed by standard techniques. For example, probes based on the sequence of the gene(s) can be individually selected and constructed by conventional techniques. Expression of the genes can be assessed, e.g., on a microarray comprising probes to one or more of the genes, e.g., an Affymetrix GENECHIP® Human Genome U133A 2.0 array, or an Affymetrix GENECHIP® Human Genome U133 Plus 2.0 (Santa Clara, Calif.). Expression of these genes can be assessed even if the sequence for a particular GenBank accession number is amended because probes specific for the amended sequence can readily be generated using well-known standard techniques.

The level of expression of these genes can be used to confirm the identity of a population of isolated placental adherent cells, to identify a population of cells as comprising at least a plurality of isolated placental adherent cells, or the like. Populations of isolated placental adherent cells, the identity of which is confirmed, can be clonal, e.g., populations of isolated placental adherent cells expanded from a single isolated placental cell, or a mixed population of stem cells, e.g., a population of cells comprising solely isolated placental adherent cells that are expanded from multiple isolated placental adherent cells, or a population of cells comprising isolated placental adherent cells, as described herein, and at least one other type of cell.

The level of expression of these genes can be used to select populations of isolated placental adherent cells. For example, a population of cells, e.g., clonally-expanded cells, may be selected if the expression of one or more of the genes listed above is significantly higher in a sample from the population of cells than in an equivalent population of mesenchymal stem cells. Such selecting can be of a population from a plurality of isolated placental cell populations, from a plurality of cell populations, the identity of which is not known, etc.

Isolated placental adherent cells can be selected on the basis of the level of expression of one or more such genes as compared to the level of expression in said one or more genes in, e.g., a mesenchymal stem cell control, for example, the level of expression in said one or more genes in an equivalent number of bone marrow-derived mesenchymal stem cells. In one embodiment, the level of expression of said one or more genes in a sample comprising an equivalent number of mesenchymal stem cells is used as a control. In another embodiment, the control, for isolated placental adherent cells tested under certain conditions, is a numeric value representing the level of expression of said one or more genes in mesenchymal stem cells under said conditions.

In certain embodiments, the placental adherent cells (e.g., placental adherent Cells®) useful in the methods provided herein, do not express CD34, as detectable by immunolocalization, after exposure to 1 to 100 ng/mL VEGF for 4 to 21 days. In a specific embodiment, said placental adherent cells are adherent to tissue culture plastic. In another specific embodiment, said population of cells induce endothelial cells to form sprouts or tube-like structures when cultured in the presence of a lymphangiogenic factor such as vascular endothelial growth factor (VEGF), epithelial growth factor (EGF), platelet derived growth factor (PDGF) or basic fibroblast growth factor (bFGF), e.g., on a substrate such as MATRIGEL™

In another aspect, the placental adherent cells provided herein, a population of cells, e.g., a population of placental adherent cells, or a population of cells wherein at least about 50%, 60%, 70%, 80%, 90%, 95% or 98% of cells in said isolated population of cells are placental adherent cells, secrete one or more, or all, of VEGF, HGF, IL-8, MCP-3, FGF2, follistatin, G-CSF, EGF, ENA-78, GRO, IL-6, MCP-1, PDGF-BB, TIMP-2, uPAR, or galectin-1, e.g., into culture medium in which the cell, or cells, are grown. In another embodiment, the placental adherent cells express increased levels of CD202b, IL-8 and/or VEGF under hypoxic conditions (e.g., less than about 5% 02) compared to normoxic conditions (e.g., about 20% or about 21% 02).

In another embodiment, any of the placental adherent cells or populations of cells comprising placental adherent cells described herein can cause the formation of sprouts or tube-like structures in a population of endothelial cells in contact with said placental derived adherent cells. In a specific embodiment, the placental adherent cells are co-cultured with human endothelial cells, which form sprouts or tube-like structures, or support the formation of endothelial cell sprouts, e.g., when cultured in the presence of extracellular matrix proteins such as collagen type I and IV, and/or angiogenic factors such as vascular endothelial growth factor (VEGF), epithelial growth factor (EGF), platelet derived growth factor (PDGF) or basic fibroblast growth factor (bFGF), e.g., in or on a substrate such as placental collagen or MATRIGEL™ for at least 4 days. In another embodiment, any of the populations of cells comprising placental derived adherent cells, described herein, secrete angiogenic factors such as vascular endothelial growth factor (VEGF), hepatocyte growth factor (HGF), platelet derived growth factor (PDGF), basic fibroblast growth factor (bFGF), or Interleukin-8 (IL-8) and thereby can induce human endothelial cells to form sprouts or tube-like structures when cultured in the presence of extracellular matrix proteins such as collagen type I and IV e.g., in or on a substrate such as placental collagen or MATRIGEL™.

In another embodiment, any of the above populations of cells comprising placental derived adherent cells (placental adherent cells) secretes angiogenic factors. In specific embodiments, the population of cells secretes vascular endothelial growth factor (VEGF), hepatocyte growth factor (HGF), platelet derived growth factor (PDGF), basic fibroblast growth factor (bFGF), and/or interleukin-8 (IL-8). In other specific embodiments, the population of cells comprising placental adherent cells secretes one or more angiogenic factors and thereby induces human endothelial cells to migrate in an in vitro wound healing assay. In other specific embodiments, the population of cells comprising placental derived adherent cells induces maturation, differentiation or proliferation of human endothelial cells, endothelial progenitors, myocytes or myoblasts.

The isolated placental adherent cells described herein display the above characteristics (e.g., combinations of cell surface markers and/or gene expression profiles) in primary culture, or during proliferation in medium comprising, e.g., DMEM-LG (Gibco), 2% fetal calf serum (FCS) (Hyclone Laboratories), 1× insulin-transferrin-selenium (ITS), 1× lenolenic-acid-bovine-serum-albumin (LA-BSA), 10⁻⁹M dexamethasone (Sigma), 10⁴M ascorbic acid 2-phosphate (Sigma), epidermal growth factor (EGF) 10 ng/ml (R&D Systems), platelet derived-growth factor (PDGF-BB) 10 ng/ml (R&D Systems), and 100 U penicillin/1000 U streptomycin.

In certain embodiments of any of the placental adherent cells disclosed herein, the cells are human. In certain embodiments of any of the placental adherent cells disclosed herein, the cellular marker characteristics or gene expression characteristics are human markers or human genes.

In another specific embodiment of said isolated placental adherent cells or populations of cells comprising the isolated placental adherent cells, said cells or population have been expanded, for example, passaged at least, about, or no more than, 1, 2, 3, 4, 5, 6, 7, 8, 9, 10, 11, 12, 13, 14, 15, 16, 17, 18, 19, or 20 times, or more, or proliferated for at least, about, or no more than, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 population doublings. In another specific embodiment of said isolated placental adherent cells or populations of cells comprising the isolated placental adherent cells, said cells or population are primary isolates. In another specific embodiment of the isolated placental adherent cells, or populations of cells comprising isolated placental adherent cells, that are disclosed herein, said isolated placental adherent cells are fetal in origin (that is, have the fetal genotype).

In certain embodiments, said isolated placental adherent cells do not differentiate during culturing in growth medium, i.e., medium formulated to promote proliferation, e.g., during proliferation in growth medium. In another specific embodiment, said isolated placental adherent cells do not require a feeder layer in order to proliferate. In another specific embodiment, said isolated placental adherent cells do not differentiate in culture in the absence of a feeder layer, solely because of the lack of a feeder cell layer.

In another embodiment, cells useful in the methods and compositions described herein are isolated placental adherent cells, wherein a plurality of said isolated placental adherent cells are positive for aldehyde dehydrogenase (ALDH), as assessed by an aldehyde dehydrogenase activity assay. Such assays are known in the art (see, e.g., Bostian and Betts, Biochem. J., 173, 787, (1978)). In a specific embodiment, said ALDH assay uses ALDEFLUOR® (Aldagen, Inc., Ashland, Oreg.) as a marker of aldehyde dehydrogenase activity. In a specific embodiment, said plurality is between about 3% and about 25% of cells in said population of cells. In another embodiment, provided herein is a population of isolated umbilical cord cells, e.g., multipotent isolated umbilical cord cells, wherein a plurality of said isolated umbilical cord cells are positive for aldehyde dehydrogenase, as assessed by an aldehyde dehydrogenase activity assay that uses ALDEFLUOR® as an indicator of aldehyde dehydrogenase activity. In a specific embodiment, said plurality is between about 3% and about 25% of cells in said population of cells. In another embodiment, said population of isolated placental adherent cells or isolated umbilical cord cells shows at least three-fold, or at least five-fold, higher ALDH activity than a population of bone marrow-derived mesenchymal stem cells having about the same number of cells and cultured under the same conditions.

In certain embodiments of any of the populations of cells comprising the isolated placental adherent cells described herein, the placental adherent cells in said populations of cells are substantially free of cells having a maternal genotype; e.g., at least 40%, 45%, 50%, 55%, 60%, 65%, 70%, 75%, 80%, 85%, 90%, 95%, 98% or 99% of the placental adherent cells in said population have a fetal genotype. In certain other embodiments of any of the populations of cells comprising the isolated placental adherent cells described herein, the populations of cells comprising said placental adherent cells are substantially free of cells having a maternal genotype; e.g., at least 40%, 45%, 50%, 55%, 60%, 65%, 70%, 75%, 80%, 85%, 90%, 95%, 98% or 99% of the cells in said population have a fetal genotype.

In a specific embodiment of any of the above isolated placental adherent cells or cell populations of isolated placental adherent cells, the karyotype of the cells, or at least about 95% or about 99% of the cells in said population, is normal. In another specific embodiment of any of the above placental adherent cells or cell populations, the cells, or cells in the population of cells, are non-maternal in origin.

Isolated placental adherent cells, or populations of isolated placental adherent cells, bearing any of the above combinations of markers, can be combined in any ratio. Any two or more of the above isolated placental cell populations can be combined to form an isolated placental cell population. For example, an population of isolated placental adherent cells can comprise a first population of isolated placental adherent cells defined by one of the marker combinations described above, and a second population of isolated placental adherent cells defined by another of the marker combinations described above, wherein said first and second populations are combined in a ratio of about 1:99, 2:98, 3:97, 4:96, 5:95, 10:90, 20:80, 30:70, 40:60, 50:50, 60:40, 70:30, 80:20, 90:10, 95:5, 96:4, 97:3, 98:2, or about 99:1. In like fashion, any three, four, five or more of the above-described isolated placental adherent cells or isolated placental adherent cells populations can be combined.

Isolated placental adherent cells useful in the methods and compositions described herein can be obtained, e.g., by disruption of placental tissue, with or without enzymatic digestion (see Section 5.4.3) or perfusion (see Section 5.4.4). For example, populations of isolated placental adherent cells can be produced according to a method comprising perfusing a mammalian placenta that has been drained of cord blood and perfused to remove residual blood; perfusing said placenta with a perfusion solution; and collecting said perfusion solution, wherein said perfusion solution after perfusion comprises a population of placental adherent cells that comprises isolated placental adherent cells; and isolating a plurality of said isolated placental adherent cells from said population of cells. In a specific embodiment, the perfusion solution is passed through both the umbilical vein and umbilical arteries and collected after it exudes from the placenta. In another specific embodiment, the perfusion solution is passed through the umbilical vein and collected from the umbilical arteries, or passed through the umbilical arteries and collected from the umbilical vein.

In various embodiments, the isolated placental adherent cells, contained within a population of cells obtained from perfusion of a placenta, are at least 50%, 60%, 70%, 80%, 90%, 95%, 99% or at least 99.5% of said population of placental adherent cells. In another specific embodiment, the isolated placental adherent cells collected by perfusion comprise fetal and maternal cells. In another specific embodiment, the isolated placental adherent cells collected by perfusion are at least 50%, 60%, 70%, 80%, 90%, 95%, 99% or at least 99.5% fetal cells.

In another specific embodiment, provided herein is a composition comprising a population of the isolated placental adherent cells, as described herein, collected by perfusion, wherein said composition comprises at least a portion of the perfusion solution used to collect the isolated placental adherent cells.

Isolated populations of the isolated placental adherent cells described herein can be produced by digesting placental tissue with a tissue-disrupting enzyme to obtain a population of placental adherent cells comprising the cells, and isolating, or substantially isolating, a plurality of the placental adherent cells from the remainder of said placental adherent cells. The whole, or any part of, the placenta can be digested to obtain the isolated placental adherent cells described herein. In specific embodiments, for example, said placental tissue can be a whole placenta, an amniotic membrane, chorion, a combination of amnion and chorion, or a combination of any of the foregoing. In other specific embodiment, the tissue-disrupting enzyme is trypsin or collagenase. In various embodiments, the isolated placental adherent cells, contained within a population of cells obtained from digesting a placenta, are at least 50%, 60%, 70%, 80%, 90%, 95%, 99% or at least 99.5% of said population of placental adherent cells.

The isolated populations of placental adherent cells described above, and populations of isolated placental adherent cells generally, can comprise about, at least, or no more than, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more of the isolated placental adherent cells. Populations of isolated placental adherent cells useful in the methods of treatment described herein comprise at least 50%, 55%, 60%, 65%, 70%, 75%, 80%, 85%, 90%, 95%, 98%, or 99% viable isolated placental adherent cells, e.g., as determinable by, e.g., trypan blue exclusion.

### 5.3.3 Growth in Culture

The growth of the isolated placental adherent cells described herein in Section 5.3, as for any mammalian cell, depends in part upon the particular medium selected for growth. Under optimum conditions, the isolated placental adherent cells typically double in number in about 1 day. During culture, the isolated placental adherent cells described herein adhere to a substrate in culture, e.g. the surface of a tissue culture container (e.g., tissue culture dish plastic, fibronectin-coated plastic, and the like) and form a monolayer.

Populations of placental adherent cells that comprise the isolated placental adherent cells described herein, when cultured under appropriate conditions, can form embryoid-like bodies, that is, three-dimensional clusters of cells grow atop the adherent cell layer. Cells within the embryoid-like bodies can express markers associated with very early stem cells, e.g., OCT-4, Nanog, SSEA3 and SSEA4. Cells within the embryoid-like bodies are typically not adherent to the culture substrate, as are the isolated placental adherent cells described herein, but tend to remain attached to the adherent cells during culture. Embryoid-like body cells are dependent upon the adherent isolated placental adherent cells for viability, as embryoid-like bodies do not form in the absence of the adherent isolated placental adherent cells. The adherent isolated placental adherent cells thus facilitate the growth of one or more embryoid-like bodies in a population of placental adherent cells that comprise the adherent isolated placental adherent cells. Without wishing to be bound by theory, the cells of the embryoid-like bodies are thought to grow on the adherent isolated placental adherent cells much as embryonic stem cells grow on a feeder layer of cells.

### 5.4 Methods of Obtaining Isolated Placental Cells

### 5.4.1 Stem Cell Collection Composition

Further provided herein are methods of collecting and isolating placental adherent cells, e.g., the isolated placental adherent cells described in Section 5.3, above. Generally, such cells are obtained from a mammalian placenta using a physiologically-acceptable solution, e.g., a cell collection composition. An exemplary cell collection composition is described in detail in related U.S. Patent Application Publication No. 2007/0190042, entitled “Improved Medium for Collecting Placental Stem Cells and Preserving Organs,” the disclosure of which is incorporated herein by reference in its entirety.

The cell collection composition can comprise any physiologically-acceptable solution suitable for the collection and/or culture of cells, e.g., the isolated placental adherent cells described herein, for example, a saline solution (e.g., phosphate-buffered saline, Kreb's solution, modified Kreb's solution, Eagle's solution, 0.9% NaCl etc.), a culture medium (e.g., DMEM, H.DMEM, etc.), and the like.

The cell collection composition can comprise one or more components that tend to preserve isolated placental adherent cells, that is, prevent the isolated placental adherent cells from dying, or delay the death of the isolated placental adherent cells, reduce the number of isolated placental adherent cells in a population of cells that die, or the like, from the time of collection to the time of culturing. Such components can be, e.g., an apoptosis inhibitor (e.g., a caspase inhibitor or JNK inhibitor); a vasodilator (e.g., magnesium sulfate, an antihypertensive drug, atrial natriuretic peptide (ANP), adrenocorticotropin, corticotropin-releasing hormone, sodium nitroprusside, hydralazine, adenosine triphosphate, adenosine, indomethacin or magnesium sulfate, a phosphodiesterase inhibitor, etc.); a necrosis inhibitor (e.g., 2-(1H-Indol-3-yl)-3-pentylamino-maleimide, pyrrolidine dithiocarbamate, or clonazepam); a TNF-α inhibitor; and/or an oxygen-carrying perfluorocarbon (e.g., perfluorooctyl bromide, perfluorodecyl bromide, etc.).

The cell collection composition can comprise one or more tissue-degrading enzymes, e.g., a metalloprotease, a serine protease, a neutral protease, an RNase, or a DNase, or the like. Such enzymes include, but are not limited to, collagenases (e.g., collagenase I, II, III or IV, a collagenase from Clostridium histolyticum, etc.); dispase, thermolysin, elastase, trypsin, LIBERASE, hyaluronidase, and the like.

The cell collection composition can comprise a bacteriocidally or bacteriostatically effective amount of an antibiotic. In certain non-limiting embodiments, the antibiotic is a macrolide (e.g., tobramycin), a cephalosporin (e.g., cephalexin, cephradine, cefuroxime, cefprozil, cefaclor, cefixime or cefadroxil), a clarithromycin, an erythromycin, a penicillin (e.g., penicillin V) or a quinolone (e.g., ofloxacin, ciprofloxacin or norfloxacin), a tetracycline, a streptomycin, etc. In a particular embodiment, the antibiotic is active against Gram(+) and/or Gram(−) bacteria, e.g., Pseudomonas aeruginosa, Staphylococcus aureus, and the like. In one embodiment, the antibiotic is gentamycin, e.g., about 0.005% to about 0.01% (w/v) in culture medium. The cell collection composition can also comprise one or more of the following compounds: adenosine (about 1 mM to about 50 mM); D-glucose (about 20 mM to about 100 mM); magnesium ions (about 1 mM to about 50 mM); a macromolecule of molecular weight greater than 20,000 daltons, in one embodiment, present in an amount sufficient to maintain endothelial integrity and cellular viability (e.g., a synthetic or naturally occurring colloid, a polysaccharide such as dextran or a polyethylene glycol present at about 25 g/l to about 100 g/l, or about 40 g/l to about 60 g/1); an antioxidant (e.g., butylated hydroxyanisole, butylated hydroxytoluene, glutathione, vitamin C or vitamin E present at about 25 μM to about 100 μM); a reducing agent (e.g., N-acetylcysteine present at about 0.1 mM to about 5 mM); an agent that prevents calcium entry into cells (e.g., verapamil present at about 2 μM to about 25 μM); nitroglycerin (e.g., about 0.05 g/L to about 0.2 g/L); an anticoagulant, in one embodiment, present in an amount sufficient to help prevent clotting of residual blood (e.g., heparin or hirudin present at a concentration of about 1000 units/1 to about 100,000 units/1); or an amiloride containing compound (e.g., amiloride, ethyl isopropyl amiloride, hexamethylene amiloride, dimethyl amiloride or isobutyl amiloride present at about 1.0 μM to about 5 μM).

### 5.4.2 Collection and Handling of Placenta

Generally, a human placenta is recovered shortly after its expulsion after birth. In a preferred embodiment, the placenta is recovered from a patient after informed consent and after a complete medical history of the patient is taken and is associated with the placenta. Preferably, the medical history continues after delivery. Such a medical history can be used to coordinate subsequent use of the placenta or the isolated placental adherent cells harvested therefrom. For example, isolated human placental adherent cells can be used, in light of the medical history, for personalized medicine for the infant associated with the placenta, or for parents, siblings or other relatives of the infant.

Prior to recovery of isolated placental adherent cells, the umbilical cord blood and placental blood are preferably removed. In certain embodiments, after delivery, the cord blood in the placenta is recovered. The placenta can be subjected to a conventional cord blood recovery process. Typically a needle or cannula is used, with the aid of gravity, to exsanguinate the placenta (see, e.g., Anderson, U.S. Pat. No. 5,372,581; Hessel et al., U.S. Pat. No. 5,415,665). The needle or cannula is usually placed in the umbilical vein and the placenta can be gently massaged to aid in draining cord blood from the placenta. Such cord blood recovery may be performed commercially, e.g., LifeBank USA, Cedar Knolls, N.J. Preferably, the placenta is gravity drained without further manipulation so as to minimize tissue disruption during cord blood recovery.

Typically, a placenta is transported from the delivery or birthing room to another location, e.g., a laboratory, for recovery of cord blood and collection of stem cells by, e.g., perfusion or tissue dissociation. The placenta is preferably transported in a sterile, thermally insulated transport device (maintaining the temperature of the placenta between 20-28° C.), for example, by placing the placenta, with clamped proximal umbilical cord, in a sterile zip-lock plastic bag, which is then placed in an insulated container. In another embodiment, the placenta is transported in a cord blood collection kit substantially as described in pending U.S. Pat. No. 7,147,626, the disclosure of which is incorporated by reference herein. Preferably, the placenta is delivered to the laboratory four to twenty-four hours following delivery. In certain embodiments, the proximal umbilical cord is clamped, preferably within 4-5 cm (centimeter) of the insertion into the placental disc prior to cord blood recovery. In other embodiments, the proximal umbilical cord is clamped after cord blood recovery but prior to further processing of the placenta.

The placenta, prior to cell collection, can be stored under sterile conditions and at either room temperature or at a temperature of 5° C. to 25° C. The placenta may be stored for a period of for a period of four to twenty-four hours, up to forty-eight hours, or longer than forty eight hours, prior to perfusing the placenta to remove any residual cord blood. In one embodiment, the placenta is harvested from between about zero hours to about two hours post-expulsion. The placenta is preferably stored in an anticoagulant solution at a temperature of 5° C. to 25° C. Suitable anticoagulant solutions are well known in the art. For example, a solution of heparin or warfarin sodium can be used. In a preferred embodiment, the anticoagulant solution comprises a solution of heparin (e.g., 1% w/w in 1:1000 solution). The exsanguinated placenta is preferably stored for no more than 36 hours before placental adherent cells are collected.

The mammalian placenta or a part thereof, once collected and prepared generally as above, can be treated in any art-known manner, e.g., can be perfused or disrupted, e.g., digested with one or more tissue-disrupting enzymes, to obtain isolated placental adherent cells.

### 5.4.3 Physical Disruption and Enzymatic Digestion of Placental Tissue

In one embodiment, stem cells are collected from a mammalian placenta by physical disruption of part of all of the organ. For example, the placenta, or a portion thereof, may be, e.g., crushed, sheared, minced, diced, chopped, macerated or the like. The tissue can then be cultured to obtain a population of isolated placental adherent cells. Typically, the placental tissue is disrupted using, e.g., culture medium, a saline solution, or a stem cell collection composition (see Section 5.4.1 and below).

The placenta can be dissected into components prior to physical disruption and/or enzymatic digestion and stem cell recovery. Isolated placental adherent cells can be obtained from all or a portion of the amniotic membrane, chorion, umbilical cord, placental cotyledons, or any combination thereof, including from a whole placenta. Preferably, isolated placental adherent cells are obtained from placental tissue comprising amnion and chorion. Typically, isolated placental adherent cells can be obtained by disruption of a small block of placental tissue, e.g., a block of placental tissue that is about 1, 2, 3, 4, 5, 6, 7, 8, 9, 10, 20, 30, 40, 50, 60, 70, 80, 90, 100, 200, 300, 400, 500, 600, 700, 800, 900 or about 1000 cubic millimeters in volume. Any method of physical disruption can be used, provided that the method of disruption leaves a plurality, more preferably a majority, and more preferably at least 60%, 70%, 80%, 90%, 95%, 98%, or 99% of the cells in said organ viable, as determinable by, e.g., trypan blue exclusion.

The isolated adherent placental adherent cells can generally be collected from a placenta, or portion thereof, at any time within about the first three days post-expulsion, but preferably between about 8 hours and about 18 hours post-expulsion.

In a specific embodiment, the disrupted tissue is cultured in tissue culture medium suitable for the proliferation of isolated placental adherent cells (see, e.g., Section 5.5, below, describing the culture of placental adherent cells, e.g., placental adherent cells).

In another specific embodiment, isolated placental adherent cells are collected by physical disruption of placental tissue, wherein the physical disruption includes enzymatic digestion, which can be accomplished by use of one or more tissue-digesting enzymes. The placenta, or a portion thereof, may also be physically disrupted and digested with one or more enzymes, and the resulting material then immersed in, or mixed into, a cell collection composition.

A preferred cell collection composition comprises one or more tissue-disruptive enzyme(s). Enzymes that can be used to disrupt placenta tissue include papain, deoxyribonucleases, serine proteases, such as trypsin, chymotrypsin, collagenase, dispase or elastase. Serine proteases may be inhibited by alpha 2 microglobulin in serum and therefore the medium used for digestion is usually serum-free. EDTA and DNase are commonly used in enzyme digestion procedures to increase the efficiency of cell recovery. The digestate is preferably diluted so as to avoid trapping cells within the viscous digest.

Any combination of tissue digestion enzymes can be used. Typical concentrations for digestion using trypsin include, 0.1% to about 2% trypsin, e.g., about 0.25% trypsin. Proteases can be used in combination, that is, two or more proteases in the same digestion reaction, or can be used sequentially in order to liberate placental adherent cells, e.g., placental stem cells and placental multipotent cells. For example, in one embodiment, a placenta, or part thereof, is digested first with an appropriate amount of collagenase I at about 1 to about 2 mg/ml for, e.g., 30 minutes, followed by digestion with trypsin, at a concentration of about 0.25%, for, e.g., 10 minutes, at 37° C. Serine proteases are preferably used consecutively following use of other enzymes.

In another embodiment, the tissue can further be disrupted by the addition of a chelator, e.g., ethylene glycol bis(2-aminoethyl ether)-N,N,N′N′-tetraacetic acid (EGTA) or ethylenediaminetetraacetic acid (EDTA) to the stem cell collection composition comprising the stem cells, or to a solution in which the tissue is disrupted and/or digested prior to isolation of the stem cells with the stem cell collection composition.

Following digestion, the digestate is washed, for example, three times with culture medium, and the washed cells are seeded into culture flasks. The cells are then isolated by differential adherence, and characterized for, e.g., viability, cell surface markers, differentiation, and the like.

It will be appreciated that where an entire placenta, or portion of a placenta comprising both fetal and maternal cells (for example, where the portion of the placenta comprises the chorion or cotyledons), the placental adherent cells isolated can comprise a mix of placental adherent cells derived from both fetal and maternal sources. Where a portion of the placenta that comprises no, or a negligible number of, maternal cells (for example, amnion), the placental adherent cells isolated therefrom will comprise almost exclusively fetal placental adherent cells (that is, placental adherent cells having the genotype of the fetus).

Placental cells, e.g., the placental adherent cells described in Section 5.3, above, can be isolated from disrupted placental tissue by differential trypsinization (see Section 5.3.5, below) followed by culture in one or more new culture containers in fresh proliferation medium, optionally followed by a second differential trypsinization step.

### 5.4.4 Placental Perfusion

Placental cells, e.g., the placental adherent cells described in Section 5.3, above, can also be obtained by perfusion of the mammalian placenta. Methods of perfusing mammalian placenta to obtain placental adherent cells are disclosed, e.g., in Hariri, U.S. Pat. Nos. 7,045,148 and 7,255,729, and in U.S. Patent Application Publication Nos. 2007/0275362 and 2007/0190042, the disclosures of each of which are incorporated herein by reference in their entireties.

Placental cells can be collected by perfusion, e.g., through the placental vasculature, using, e.g., a cell collection composition as a perfusion solution. In one embodiment, a mammalian placenta is perfused by passage of perfusion solution through either or both of the umbilical artery and umbilical vein. The flow of perfusion solution through the placenta may be accomplished using, e.g., gravity flow into the placenta. Preferably, the perfusion solution is forced through the placenta using a pump, e.g., a peristaltic pump. The umbilical vein can be, e.g., cannulated with a cannula, e.g., a TEFLON® or plastic cannula, that is connected to a sterile connection apparatus, such as sterile tubing. The sterile connection apparatus is connected to a perfusion manifold.

In preparation for perfusion, the placenta is preferably oriented (e.g., suspended) in such a manner that the umbilical artery and umbilical vein are located at the highest point of the placenta. The placenta can be perfused by passage of a perfusion fluid through the placental vasculature and surrounding tissue. The placenta can also be perfused by passage of a perfusion fluid into the umbilical vein and collection from the umbilical arteries, or passage of a perfusion fluid into the umbilical arteries and collection from the umbilical vein.

In one embodiment, for example, the umbilical artery and the umbilical vein are connected simultaneously, e.g., to a pipette that is connected via a flexible connector to a reservoir of the perfusion solution. The perfusion solution is passed into the umbilical vein and artery. The perfusion solution exudes from and/or passes through the walls of the blood vessels into the surrounding tissues of the placenta, and is collected in a suitable open vessel from the surface of the placenta that was attached to the uterus of the mother during gestation. The perfusion solution may also be introduced through the umbilical cord opening and allowed to flow or percolate out of openings in the wall of the placenta which interfaced with the maternal uterine wall. Placental cells that are collected by this method, which can be referred to as a “pan” method, are typically a mixture of fetal and maternal cells.

In another embodiment, the perfusion solution is passed through the umbilical veins and collected from the umbilical artery, or is passed through the umbilical artery and collected from the umbilical veins. Placental cells collected by this method, which can be referred to as a “closed circuit” method, are typically almost exclusively fetal.

It will be appreciated that perfusion using the pan method, that is, whereby perfusate is collected after it has exuded from the maternal side of the placenta, results in a mix of fetal and maternal cells. As a result, the cells collected by this method can comprise a mixed population of placental adherent cells, e.g., placental stem cells or placental multipotent cells, of both fetal and maternal origin. In contrast, perfusion solely through the placental vasculature in the closed circuit method, whereby perfusion fluid is passed through one or two placental vessels and is collected solely through the remaining vessel(s), results in the collection of a population of placental adherent cells almost exclusively of fetal origin.

The closed circuit perfusion method can, in one embodiment, be performed as follows. A post-partum placenta is obtained within about 48 hours after birth. The umbilical cord is clamped and cut above the clamp. The umbilical cord can be discarded, or can processed to recover, e.g., umbilical cord stem cells, and/or to process the umbilical cord membrane for the production of a biomaterial. The amniotic membrane can be retained during perfusion, or can be separated from the chorion, e.g., using blunt dissection with the fingers. If the amniotic membrane is separated from the chorion prior to perfusion, it can be, e.g., discarded, or processed, e.g., to obtain stem cells by enzymatic digestion, or to produce, e.g., an amniotic membrane biomaterial, e.g., the biomaterial described in U.S. Application Publication No. 2004/0048796, the disclosure of which is incorporated by reference herein in its entirety. After cleaning the placenta of all visible blood clots and residual blood, e.g., using sterile gauze, the umbilical cord vessels are exposed, e.g., by partially cutting the umbilical cord membrane to expose a cross-section of the cord. The vessels are identified, and opened, e.g., by advancing a closed alligator clamp through the cut end of each vessel. The apparatus, e.g., plastic tubing connected to a perfusion device or peristaltic pump, is then inserted into each of the placental arteries. The pump can be any pump suitable for the purpose, e.g., a peristaltic pump. Plastic tubing, connected to a sterile collection reservoir, e.g., a blood bag such as a 250 mL collection bag, is then inserted into the placental vein. Alternatively, the tubing connected to the pump is inserted into the placental vein, and tubes to a collection reservoir(s) are inserted into one or both of the placental arteries. The placenta is then perfused with a volume of perfusion solution, e.g., about 750 ml of perfusion solution. Cells in the perfusate are then collected, e.g., by centrifugation. In certain embodiments, the placenta is perfused with perfusion solution, e.g., 100-300 mL perfusion solution, to remove residual blood prior to perfusion to collect placental adherent cells, e.g., placental stem cells and/or placental multipotent cells. In another embodiment, the placenta is not perfused with perfusion solution to remove residual blood prior to perfusion to collect placental adherent cells.

In one embodiment, the proximal umbilical cord is clamped during perfusion, and more preferably, is clamped within 4-5 cm (centimeter) of the cord's insertion into the placental disc.

The first collection of perfusion fluid from a mammalian placenta during the exsanguination process is generally colored with residual red blood cells of the cord blood and/or placental blood. The perfusion fluid becomes more colorless as perfusion proceeds and the residual cord blood cells are washed out of the placenta. Generally from 30 to 100 ml (milliliter) of perfusion fluid is adequate to initially exsanguinate the placenta, but more or less perfusion fluid may be used depending on the observed results.

The volume of perfusion liquid used to isolate placental adherent cells may vary depending upon the number of cells to be collected, the size of the placenta, the number of collections to be made from a single placenta, etc. In various embodiments, the volume of perfusion liquid may be from 50 mL to 5000 mL, 50 mL to 4000 mL, 50 mL to 3000 mL, 100 mL to 2000 mL, 250 mL to 2000 mL, 500 mL to 2000 mL, or 750 mL to 2000 mL. Typically, the placenta is perfused with 700-800 mL of perfusion liquid following exsanguination.

The placenta can be perfused a plurality of times over the course of several hours or several days. Where the placenta is to be perfused a plurality of times, it may be maintained or cultured under aseptic conditions in a container or other suitable vessel, and perfused with the cell collection composition, or a standard perfusion solution (e.g., a normal saline solution such as phosphate buffered saline (“PBS”)) with or without an anticoagulant (e.g., heparin, warfarin sodium, coumarin, bishydroxycoumarin), and/or with or without an antimicrobial agent (e.g., β-mercaptoethanol (0.1 mM); antibiotics such as streptomycin (e.g., at 40-100 μg/ml), penicillin (e.g., at 40 U/ml), amphotericin B (e.g., at 0.5 μg/ml). In one embodiment, an isolated placenta is maintained or cultured for a period of time without collecting the perfusate, such that the placenta is maintained or cultured for 1, 2, 3, 4, 5, 6, 7, 8, 9, 10, 11, 12, 13, 14, 15, 16, 17, 18, 19, 20, 21, 22, 23, or 24 hours, or 2 or 3 or more days before perfusion and collection of perfusate. The perfused placenta can be maintained for one or more additional time(s), e.g., 1, 2, 3, 4, 5, 6, 7, 8, 9, 10, 11, 12, 13, 14, 15, 16, 17, 18, 19, 20, 21, 22, 23, 24 or more hours, and perfused a second time with, e.g., 700-800 mL perfusion fluid. The placenta can be perfused 1, 2, 3, 4, 5 or more times, for example, once every 1, 2, 3, 4, 5 or 6 hours. In a preferred embodiment, perfusion of the placenta and collection of perfusion solution, e.g., cell collection composition, is repeated until the number of recovered nucleated cells falls below 100 cells/ml. The perfusates at different time points can be further processed individually to recover time-dependent populations of cells, e.g., stem cells. Perfusates from different time points can also be pooled. In a preferred embodiment, placental adherent cells are collected at a time or times between about 8 hours and about 18 hours post-expulsion.

Perfusion preferably results in the collection of significantly more placental adherent cells than the number obtainable from a mammalian placenta not perfused with said solution, and not otherwise treated to obtain placental adherent cells (e.g., by tissue disruption, e.g., enzymatic digestion). In this context, “significantly more” means at least 10% more. Perfusion yields significantly more placental adherent cells than, e.g., the number of placental adherent cells isolatable from culture medium in which a placenta, or portion thereof, has been cultured.

Placental cells can be isolated from placenta by perfusion with a solution comprising one or more proteases or other tissue-disruptive enzymes. In a specific embodiment, a placenta or portion thereof (e.g., amniotic membrane, amnion and chorion, placental lobule or cotyledon, umbilical cord, or combination of any of the foregoing) is brought to 25-37° C., and is incubated with one or more tissue-disruptive enzymes in 200 mL of a culture medium for 30 minutes. Cells from the perfusate are collected, brought to 4° C., and washed with a cold inhibitor mix comprising 5 mM EDTA, 2 mM dithiothreitol and 2 mM beta-mercaptoethanol. The placental adherent cells are washed after several minutes with a cold (e.g., 4° C.) stem cell collection composition.

### 5.4.5 Isolation, Sorting, and Characterization of Placental Cells

The isolated placental adherent cells, e.g., the cells described in Section 5.3, above, whether obtained by perfusion or physical disruption, e.g., by enzymatic digestion, can initially be purified from (i.e., be isolated from) other cells by Ficoll gradient centrifugation. Such centrifugation can follow any standard protocol for centrifugation speed, etc. In one embodiment, for example, cells collected from the placenta are recovered from perfusate by centrifugation at 5000×g for 15 minutes at room temperature, which separates cells from, e.g., contaminating debris and platelets. In another embodiment, placental perfusate is concentrated to about 200 ml, gently layered over Ficoll, and centrifuged at about 1100×g for 20 minutes at 22° C., and the low-density interface layer of cells is collected for further processing.

Cell pellets can be resuspended in fresh stem cell collection composition, or a medium suitable for cell maintenance, e.g., stem cell maintenance, for example, IMDM serum-free medium containing 2 U/ml heparin and 2 mM EDTA (GibcoBRL, N.Y.). The total mononuclear cell fraction can be isolated, e.g., using Lymphoprep (Nycomed Pharma, Oslo, Norway) according to the manufacturer's recommended procedure.

Placental cells obtained by perfusion or digestion can, for example, be further, or initially, isolated by differential trypsinization using, e.g., a solution of 0.05% trypsin with 0.2% EDTA (Sigma, St. Louis Mo.). Differential trypsinization is possible because the isolated placental adherent cells, which are tissue culture plastic-adherent, typically detach from the plastic surfaces within about five minutes whereas other adherent populations typically require more than 20-30 minutes incubation. The detached placental adherent cells can be harvested following trypsinization and trypsin neutralization, using, e.g., Trypsin Neutralizing Solution (TNS, Cambrex). In one embodiment of isolation of adherent cells, aliquots of, for example, about 5-10×10⁶ cells are placed in each of several T-75 flasks, preferably fibronectin-coated T75 flasks. In such an embodiment, the cells can be cultured with commercially available Mesenchymal Stem Cell Growth Medium (MSCGM) (Cambrex), and placed in a tissue culture incubator (37° C., 5% CO₂). After 10 to 15 days, non-adherent cells are removed from the flasks by washing with PBS. The PBS is then replaced by MSCGM. Flasks are preferably examined daily for the presence of various adherent cell types and in particular, for identification and expansion of clusters of fibroblastoid cells.

The number and type of cells collected from a mammalian placenta can be monitored, for example, by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. For example, using antibodies to CD34, one can determine, using the techniques above, whether a cell comprises a detectable amount of CD34; if so, the cell is CD34⁺. Likewise, if a cell produces enough OCT-4 RNA to be detectable by RT-PCR, or significantly more OCT-4 RNA than an adult cell, the cell is OCT-4⁺. Antibodies to cell surface markers (e.g., CD markers such as CD34) and the sequence of stem cell-specific genes, such as OCT-4, are well-known in the art.

Placental cells, particularly cells that have been isolated by Ficoll separation, differential adherence, or a combination of both, may be sorted using a fluorescence activated cell sorter (FACS). Fluorescence activated cell sorting (FACS) is a well-known method for separating particles, including cells, based on the fluorescent properties of the particles (Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In one sorting scheme, cells from placenta, e.g., placental adherent cells are sorted on the basis of expression of one or more of the markers CD34, CD38, CD44, CD45, CD73, CD105, OCT-4 and/or HLA-G. This can be accomplished in connection with procedures to select such cells on the basis of their adherence properties in culture. For example, tissue culture plastic adherence selection can be accomplished before or after sorting on the basis of marker expression. In one embodiment, for example, cells are sorted first on the basis of their expression of CD34; CD34⁻ cells are retained, and CD34⁻ cells that are additionally CD200⁺ and HLA-G⁻ are separated from all other CD34⁻ cells. In another embodiment, cells from placenta are sorted based on their expression of markers CD200 and/or HLA-G; for example, cells displaying CD200 and lacking HLA-G are isolated for further use. Cells that express, e.g., CD200 and/or lack, e.g., HLA-G can, in a specific embodiment, be further sorted based on their expression of CD73 and/or CD105, or epitopes recognized by antibodies SH2, SH3 or SH4, or lack of expression of CD34, CD38 or CD45. For example, in another embodiment, placental adherent cells are sorted by expression, or lack thereof, of CD200, HLA-G, CD73, CD105, CD34, CD38 and CD45, and placental adherent cells that are CD200⁺, HLA-G⁻, CD73⁺, CD105⁺, CD34⁻, CD38⁻ and CD45⁻ are isolated from other placental adherent cells for further use.

In specific embodiments of any of the above embodiments of sorted placental adherent cells, at least 50%, 60%, 70%, 80%, 90% or 95% of the cells in a cell population remaining after sorting are said isolated placental adherent cells. Placental cells can be sorted by one or more of any of the markers described in Section 5.3, above.

In a specific embodiment, for example, placental adherent cells that are (1) adherent to tissue culture plastic, and (2) CD10⁺, CD34⁻ and CD105⁺ are sorted from (i.e., isolated from) other placental adherent cells. In another specific embodiment, placental adherent cells that are (1) adherent to tissue culture plastic, and (2) CD10⁺, CD34⁻, CD105⁺ and CD200⁺ are sorted from (i.e., isolated from) other placental adherent cells. In another specific embodiment, placental adherent cells that are (1) adherent to tissue culture plastic, and (2) CD10⁺, CD34⁻, CD45⁻, CD90⁺, CD105⁺ and CD200⁺ are sorted from (i.e., isolated from) other placental adherent cells.

With respect to nucleotide sequence-based detection of placental adherent cells, sequences for the markers listed herein are readily available in publicly-available databases such as GenBank or EMBL.

With respect to antibody-mediated detection and sorting of placental adherent cells, e.g., placental stem cells or placental multipotent cells, any antibody, specific for a particular marker, can be used, in combination with any fluorophore or other label suitable for the detection and sorting of cells (e.g., fluorescence-activated cell sorting). Antibody/fluorophore combinations to specific markers include, but are not limited to, fluorescein isothiocyanate (FITC) conjugated monoclonal antibodies against HLA-G (available from Serotec, Raleigh, N.C.), CD10 (available from BD Immunocytometry Systems, San Jose, Calif.), CD44 (available from BD Biosciences Pharmingen, San Jose, Calif.), and CD105 (available from R&D Systems Inc., Minneapolis, Minn.); phycoerythrin (PE) conjugated monoclonal antibodies against CD44, CD200, CD117, and CD13 (BD Biosciences Pharmingen); phycoerythrin-Cy7 (PE Cy7) conjugated monoclonal antibodies against CD33 and CD10 (BD Biosciences Pharmingen); allophycocyanin (APC) conjugated streptavidin and monoclonal antibodies against CD38 (BD Biosciences Pharmingen); and Biotinylated CD90 (BD Biosciences Pharmingen). Other antibodies that can be used include, but are not limited to, CD133-APC (Miltenyi), KDR-Biotin (CD309, Abcam), CytokeratinK-Fitc (Sigma or Dako), HLA ABC-Fitc (BD), HLA DR,DQ,DP-PE (BD), β-2-microglobulin-PE (BD), CD80-PE (BD) and CD86-APC (BD). Other antibody/label combinations that can be used include, but are not limited to, CD45-PerCP (peridin chlorophyll protein); CD44-PE; CD19-PE; CD10-F (fluorescein); HLA-G-F and 7-amino-actinomycin-D (7-AAD); HLA-ABC-F; and the like. This list is not exhaustive, and other antibodies from other suppliers are also commercially available.

The isolated placental adherent cells provided herein can be assayed for CD117 or CD133 using, for example, phycoerythrin-Cy5 (PE Cy5) conjugated streptavidin and biotin conjugated monoclonal antibodies against CD117 or CD133; however, using this system, the cells can appear to be positive for CD117 or CD133, respectively, because of a relatively high background.

The isolated placental adherent cells can be labeled with an antibody to a single marker and detected and/sorted. Placental cells can also be simultaneously labeled with multiple antibodies to different markers.

In another embodiment, magnetic beads can be used to separate cells. The cells may be sorted using a magnetic activated cell sorting (MACS) technique, a method for separating particles based on their ability to bind magnetic beads (0.5-100 μm diameter). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

Isolated placental adherent cells can also be characterized and/or sorted based on cell morphology and growth characteristics. For example, isolated placental adherent cells can be characterized as having, and/or selected on the basis of, e.g., a fibroblastoid appearance in culture. The isolated placental adherent cells can also be characterized as having, and/or be selected, on the basis of their ability to form embryoid-like bodies. In one embodiment, for example, placental adherent cells that are fibroblastoid in shape, express CD73 and CD105, and produce one or more embryoid-like bodies in culture are isolated from other placental adherent cells. In another embodiment, OCT-4⁺ placental adherent cells that produce one or more embryoid-like bodies in culture are isolated from other placental adherent cells.

In another embodiment, isolated placental adherent cells can be identified and characterized by a colony forming unit assay. Colony forming unit assays are commonly known in the art, such as MESENCULT™ medium (Stem Cell Technologies, Inc., Vancouver British Columbia).

The isolated placental adherent cells can be assessed for viability, proliferation potential, and longevity using standard techniques known in the art, such as trypan blue exclusion assay, fluorescein diacetate uptake assay, propidium iodide uptake assay (to assess viability); and thymidine uptake assay, MTT (3-(4,5-Dimethylthiazol-2-yl)-2,5-diphenyltetrazolium bromide) cell proliferation assay (to assess proliferation). Longevity may be determinable by methods well known in the art, such as by determining the maximum number of population doubling in an extended culture.

Isolated placental adherent cells, e.g., the isolated placental adherent cells described in Section 5.3, above, can also be separated from other placental adherent cells using other techniques known in the art, e.g., selective growth of desired cells (positive selection), selective destruction of unwanted cells (negative selection); separation based upon differential cell agglutinability in the mixed population as, for example, with soybean agglutinin; freeze-thaw procedures; filtration; conventional and zonal centrifugation; centrifugal elutriation (counter-streaming centrifugation); unit gravity separation; countercurrent distribution; electrophoresis; and the like.

### 5.5 Culture of Isolated Placental Cells

### 5.5.1 Culture Media

Isolated placental adherent cells, or populations of isolated placental adherent cells, or cells or placental tissue from which placental adherent cells grow out, can be used to initiate, or seed, cell cultures. Cells are generally transferred to sterile tissue culture vessels either uncoated or coated with extracellular matrix or ligands such as laminin, collagen (e.g., native or denatured), gelatin, fibronectin, ornithine, vitronectin, polylysine, CELLSTART™, and/or extracellular membrane protein (e.g., MATRIGEL® (BD Discovery Labware, Bedford, Mass.)), or other suitable biomolecule or synthetic mimetic agent.

Isolated placental adherent cells can be cultured in any medium, and under any conditions, recognized in the art as acceptable for the culture of cells, e.g., stem cells. Preferably, the culture medium comprises serum. The isolated placental adherent cells can be cultured in, for example, DMEM-LG (Dulbecco's Modified Essential Medium, low glucose)/MCDB 201 (chick fibroblast basal medium) containing ITS (insulin-transferrin-selenium), LA+BSA (linoleic acid-bovine serum albumin), dexamethasone L-ascorbic acid, PDGF, EGF, IGF-1, and penicillin/streptomycin; DMEM-HG (high glucose) comprising 10% fetal bovine serum (FBS); DMEM-HG comprising 15% FBS; IMDM (Iscove's modified Dulbecco's medium) comprising 10% FBS, 10% horse serum, and hydrocortisone; M199 comprising 1% to 20% FBS, EGF, and heparin; .alpha.-MEM (minimal essential medium) comprising 10% FBS, GLUTAMAX™ and gentamicin; DMEM comprising 10% FBS, GLUTAMAX™ and gentamicin, etc.

Other media in that can be used to culture placental adherent cells include DMEM (high or low glucose), Eagle's basal medium, Ham's F10 medium (F10), Ham's F-12 medium (F12), Iscove's modified Dulbecco's medium, Mesenchymal Stem Cell Growth Medium (MSCGM), Liebovitz's L-15 medium, MCDB, DMEM/F12, RPMI 1640, advanced DMEM (Gibco), DMEM/MCDB201 (Sigma), and CELL-GRO FREE.

The culture medium can be supplemented with one or more components including, for example, serum (e.g., fetal bovine serum (FBS), preferably about 2-15% (v/v); equine (horse) serum (ES); human serum (HS)); beta-mercaptoethanol (BME), preferably about 0.001% (v/v); one or more growth factors, for example, platelet-derived growth factor (PDGF), epidermal growth factor (EGF), basic fibroblast growth factor (bFGF), insulin-like growth factor-1 (IGF-1), leukemia inhibitory factor (LIF), vascular endothelial growth factor (VEGF), and erythropoietin (EPO); amino acids, including L-valine; and one or more antibiotic and/or antimycotic agents to control microbial contamination, such as, for example, penicillin G, streptomycin sulfate, amphotericin B, gentamicin, and nystatin, either alone or in combination.

The isolated placental adherent cells can be cultured in standard tissue culture conditions, e.g., in tissue culture dishes or multiwell plates. The isolated placental adherent cells can also be cultured using a hanging drop method. In this method, isolated placental adherent cells are suspended at about 1×10⁴ cells per mL in about 5 mL of medium, and one or more drops of the medium are placed on the inside of the lid of a tissue culture container, e.g., a 100 mL Petri dish. The drops can be, e.g., single drops, or multiple drops from, e.g., a multichannel pipetter. The lid is carefully inverted and placed on top of the bottom of the dish, which contains a volume of liquid, e.g., sterile PBS sufficient to maintain the moisture content in the dish atmosphere, and the stem cells are cultured.

In one embodiment, isolated placental adherent cells are cultured in the presence of a compound that acts to maintain an undifferentiated phenotype in the isolated placental adherent cells. In a specific embodiment, the compound is a substituted 3,4-dihydropyridimol[4,5-d]pyrimidine. In another specific embodiment, the compound is a compound having the following chemical structure:

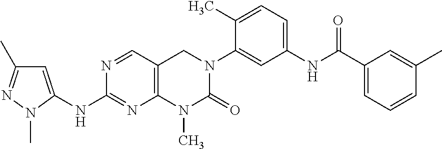

The compound can be contacted with isolated placental adherent cells, or a population of isolated placental adherent cells, at a concentration of, for example, between about 1 μM to about 10 μM.

### 5.5.2 Expansion and Proliferation of Placental Cells

Once an isolated placental cell, or population of isolated placental adherent cells (e.g., a placental cell or population of placental adherent cells separated from at least 50% of the placental adherent cells with which the stem cell or population of stem cells is normally associated in vivo), the cell or population of cells can be proliferated and expanded in vitro. For example, a population of the isolated placental adherent cells can be cultured in tissue culture containers, e.g., dishes, flasks, multiwell plates, or the like, for a sufficient time for the cells to proliferate to 70-90% confluence, that is, until the cells and their progeny occupy 70-90% of the culturing surface area of the tissue culture container.

The isolated placental adherent cells can be seeded in culture vessels at a density that allows cell growth. For example, the cells may be seeded at low density (e.g., about 1,000 to about 5,000 cells/cm²) to high density (e.g., about 50,000 or more cells/cm²). In a preferred embodiment, the cells are cultured in the presence of about 0 to about 5 percent by volume CO₂ in air. In some preferred embodiments, the cells are cultured at about 2 to about 25 percent 02 in air, preferably about 5 to about 20 percent 02 in air. The cells preferably are cultured at about 25° C. to about 40° C., preferably 37° C. The cells are preferably cultured in an incubator. The culture medium can be static or agitated, for example, using a bioreactor. Placental cells, e.g., placental stem cells or placental multipotent cells, preferably are grown under low oxidative stress (e.g., with addition of glutathione, ascorbic acid, catalase, tocopherol, N-acetylcysteine, or the like).

Once confluence of less than 100%, for example, 70% to 90% is obtained, the cells may be passaged. For example, the cells can be enzymatically treated, e.g., trypsinized, using techniques well-known in the art, to separate them from the tissue culture surface. After removing the cells by pipetting and counting the cells, about 10,000-100,000 cells/cm² are passaged to a new culture container containing fresh culture medium. Typically, the new medium is the same type of medium from which the isolated placental adherent cells were removed. The isolated placental adherent cells can be passaged about, at least, or no more than 1, 2, 3, 4, 5, 6, 7, 8, 9, 10, 12, 14, 16, 18, or 20 times, or more.

### 5.5.3 Populations of Isolated Placental Cells

Also provided herein are populations of isolated placental adherent cells, e.g., the isolated placental adherent cells described in Section 5.3, above, useful in the methods and compositions described herein. Populations of isolated placental adherent cells can be isolated directly from one or more placentas; that is, the cell population can be a population of placental adherent cells comprising the isolated placental adherent cells, wherein the isolated placental adherent cells are obtained from, or contained within, perfusate, or obtained from, or contained within, disrupted placental tissue, e.g., placental tissue digestate (that is, the collection of cells obtained by enzymatic digestion of a placenta or part thereof). The isolated placental adherent cells described herein can also be cultured and expanded to produce populations of the isolated placental adherent cells. Populations of placental adherent cells comprising the isolated placental adherent cells can also be cultured and expanded to produce placental cell populations.

Placental cell populations useful in the methods of treatment provided herein comprise the isolated placental adherent cells, for example, the isolated placental adherent cells as described in Section 5.3 herein. In various embodiments, at least 10%, 20%, 30%, 40%, 50%, 60%, 70%, 80%, 90%, 95%, or 99% of the cells in a placental cell population are the isolated placental adherent cells. That is, a population of the isolated placental adherent cells can comprise, e.g., as much as 1%, 5%, 10%, 20%, 30%, 40%, 50%, 60%, 70%, 80%, 90% cells that are not the isolated placental adherent cells.

Isolated placental cell populations useful in the methods and compositions described herein can be produced by, e.g., selecting isolated placental adherent cells, whether derived from enzymatic digestion or perfusion, that express particular markers and/or particular culture or morphological characteristics. In one embodiment, for example, provided herein is a method of producing a cell population by selecting placental adherent cells that (a) adhere to a substrate, and (b) express CD200 and lack expression of HLA-G; and isolating said cells from other cells to form a cell population. In another embodiment, a cell population is produced by selecting placental adherent cells that express CD200 and lack expression of HLA-G, and isolating said cells from other cells to form a cell population. In another embodiment, a cell population is produced by selecting placental adherent cells that (a) adhere to a substrate, and (b) express CD73, CD105, and CD200; and isolating said cells from other cells to form a cell population. In another embodiment, a cell population is produced by identifying placental adherent cells that express CD73, CD105, and CD200, and isolating said cells from other cells to form a cell population. In another embodiment, a cell population is produced by selecting placental adherent cells that (a) adhere to a substrate and (b) express CD200 and OCT-4; and isolating said cells from other cells to form a cell population. In another embodiment, a cell population is produced by selecting placental adherent cells that express CD200 and OCT-4, and isolating said cells from other cells to form a cell population. In another embodiment, a cell population is produced by selecting placental adherent cells that (a) adhere to a substrate, (b) express CD73 and CD105, and (c) facilitate the formation of one or more embryoid-like bodies in a population of placental adherent cells comprising said stem cell when said population is cultured under conditions that allow for the formation of an embryoid-like body; and isolating said cells from other cells to form a cell population. In another embodiment, a cell population is produced by selecting placental adherent cells that express CD73 and CD105, and facilitate the formation of one or more embryoid-like bodies in a population of placental adherent cells comprising said stem cell when said population is cultured under conditions that allow for the formation of an embryoid-like body, and isolating said cells from other cells to form a cell population. In another embodiment, a cell population is produced by selecting placental adherent cells that (a) adhere to a substrate, and (b) express CD73 and CD105, and lack expression of HLA-G; and isolating said cells from other cells to form a cell population. In another embodiment, a cell population is produced by selecting placental adherent cells that express CD73 and CD105 and lack expression of HLA-G, and isolating said cells from other cells to form a cell population. In another embodiment, the method of producing a cell population comprises selecting placental adherent cells that (a) adhere to a substrate, (b) express OCT-4, and (c) facilitate the formation of one or more embryoid-like bodies in a population of placental adherent cells comprising said stem cell when said population is cultured under conditions that allow for the formation of an embryoid-like body; and isolating said cells from other cells to form a cell population. In another embodiment, a cell population is produced by selecting placental adherent cells that express OCT-4, and facilitate the formation of one or more embryoid-like bodies in a population of placental adherent cells comprising said stem cell when said population is cultured under conditions that allow for the formation of an embryoid-like body, and isolating said cells from other cells to form a cell population.

In another embodiment, a cell population is produced by selecting placental adherent cells that (a) adhere to a substrate, and (b) express CD10 and CD105, and do not express CD34; and isolating said cells from other cells to form a cell population. In another embodiment, a cell population is produced by selecting placental adherent cells that express CD10 and CD105, and do not express CD34, and isolating said cells from other cells to form a cell population. In another embodiment, a cell population is produced by selecting placental adherent cells that (a) adhere to a substrate, and (b) express CD10, CD105, and CD200, and do not express CD34; and isolating said cells from other cells to form a cell population. In another embodiment, a cell population is produced by selecting placental adherent cells that express CD10, CD105, and CD200, and do not express CD34, and isolating said cells from other cells to form a cell population. In another specific embodiment, a cell population is produced by selecting placental adherent cells that (a) adhere to a substrate, and (b) express CD10, CD90, CD105 and CD200, and do not express CD34 and CD45; and isolating said cells from other cells to form a cell population. In another specific embodiment, a cell population is produced by selecting placental adherent cells that express CD10, CD90, CD105 and CD200, and do not express CD34 and CD45, and isolating said cells from other cells to form a cell population.

Selection of cell populations comprising placental adherent cells having any of the marker combinations described in Section 5.3, above, can be isolated or obtained in similar fashion.

In any of the above embodiments, selection of the isolated cell populations can additionally comprise selecting placental adherent cells that express ABC-p (a placenta-specific ABC transporter protein; see, e.g., Allikmets et al., Cancer Res. 58(23):5337-9 (1998)). The method can also comprise selecting cells exhibiting at least one characteristic specific to, e.g., a mesenchymal stem cell, for example, expression of CD44, expression of CD90, or expression of a combination of the foregoing.

In the above embodiments, the substrate can be any surface on which culture and/or selection of cells, e.g., isolated placental adherent cells, can be accomplished. Typically, the substrate is plastic, e.g., tissue culture dish or multiwell plate plastic. Tissue culture plastic can be coated with a biomolecule, e.g., laminin or fibronectin.

Cells, e.g., isolated placental adherent cells, can be selected for a placental cell population by any means known in the art of cell selection. For example, cells can be selected using an antibody or antibodies to one or more cell surface markers, for example, in flow cytometry or FACS. Selection can be accomplished using antibodies in conjunction with magnetic beads. Antibodies that are specific for certain stem cell-related markers are known in the art. For example, antibodies to OCT-4 (Abcam, Cambridge, Mass.), CD200 (Abcam), HLA-G (Abeam), CD73 (BD Biosciences Pharmingen, San Diego, Calif), CD105 (Abeam; BioDesign International, Saco, Me.), etc. Antibodies to other markers are also available commercially, e.g., CD34, CD38 and CD45 are available from, e.g., StemCell Technologies or BioDesign International.

The isolated placental cell populations can comprise placental adherent cells that are not stem cells, or cells that are not placental adherent cells.

The isolated cell populations comprising placental derived adherent cells described herein can comprise a second cell type, e.g., placental adherent cells that are not placental derived adherent cells, or, e.g., cells that are not placental adherent cells. For example, an isolated population of placental derived adherent cells can comprise, e.g., can be combined with, a population of a second type of cells, wherein said second type of cell are, e.g., embryonic stem cells, blood cells (e.g., placental blood, placental blood cells, umbilical cord blood, umbilical cord blood cells, peripheral blood, peripheral blood cells, nucleated cells from placental blood, umbilical cord blood, or peripheral blood, and the like), stem cells isolated from blood (e.g., stem cells isolated from placental blood, umbilical cord blood or peripheral blood), nucleated cells from placental perfusate, e.g., total nucleated cells from placental perfusate; umbilical cord stem cells, populations of blood-derived nucleated cells, bone marrow-derived mesenchymal stromal cells, bone marrow-derived mesenchymal stem cells, bone marrow-derived hematopoietic stem cells, crude bone marrow, adult (somatic) stem cells, populations of stem cells contained within tissue, cultured cells, e.g., cultured stem cells, populations of fully-differentiated cells (e.g., chondrocytes, fibroblasts, amniotic cells, osteoblasts, muscle cells, cardiac cells, etc.), pericytes, and the like. In a specific embodiment, a population of cells comprising placental derived adherent cells comprises placental stem cells or stem cells from umbilical cord. In certain embodiments in which the second type of cell is blood or blood cells, erythrocytes have been removed from the population of cells.

In a specific embodiment, the second type of cell is a hematopoietic stem cell. Such hematopoietic stem cells can be, for example, contained within unprocessed placental, umbilical cord blood or peripheral blood; in total nucleated cells from placental blood, umbilical cord blood or peripheral blood; in an isolated population of CD34⁺ cells from placental blood, umbilical cord blood or peripheral blood; in unprocessed bone marrow; in total nucleated cells from bone marrow; in an isolated population of CD34⁺ cells from bone marrow, or the like.

In another embodiment, an isolated population of placental derived adherent cells is combined with a plurality of adult or progenitor cells from the vascular system. In various embodiments, the cells are endothelial cells, endothelial progenitor cells, myocytes, cardiomyocytes, pericytes, angioblasts, myoblasts or cardiomyoblasts.

In a another embodiment, the second cell type is a non-embryonic cell type manipulated in culture in order to express markers of pluripotency and functions associated with embryonic stem cells.

In specific embodiments of the above isolated populations of placental derived adherent cells, either or both of the placental derived adherent cells and cells of a second type are autologous, or are allogeneic, to an intended recipient of the cells.

In another specific embodiment, the composition comprises placental derived adherent cells, and embryonic stem cells. In another specific embodiment, the composition comprises placental derived adherent cells and mesenchymal stromal or stem cells, e.g., bone marrow-derived mesenchymal stromal or stem cells. In another specific embodiment, the composition comprises bone marrow-derived hematopoietic stem cells. In another specific embodiment, the composition comprises placental derived adherent cells and hematopoietic progenitor cells, e.g., hematopoietic progenitor cells from bone marrow, fetal blood, umbilical cord blood, placental blood, and/or peripheral blood. In another specific embodiment, the composition comprises placental derived adherent cells and somatic stem cells. In a more specific embodiment, said somatic stem cell is a neural stem cell, a hepatic stem cell, a pancreatic stem cell, an endothelial stem cell, a cardiac stem cell, or a muscle stem cell.

In other specific embodiments, the second type of cells comprise about, at least, or no more than, 10%, 15%, 20%, 25%, 30%, 35%, 40%, 45%, or 50% of cells in said population. In other specific embodiments, the placental adherent cell in said composition comprise at least 50%, 55%, 60%, 65%, 70%, 75%, 80%, 85% or 90% of cells in said composition. In other specific embodiments, the placental derived adherent cells comprise about, at least, or no more than, 10%, 15%, 20%, 25%, 30%, 35%, 40%, or 45% of cells in said population.

Cells in an isolated population of placental derived adherent cells can be combined with a plurality of cells of another type, e.g., with a population of stem cells, in a ratio of about 100,000,000:1, 50,000,000:1, 20,000,000:1, 10,000,000:1, 5,000,000:1, 2,000,000:1, 1,000,000:1, 500,000:1, 200,000:1, 100,000:1, 50,000:1, 20,000:1, 10,000:1, 5,000:1, 2,000:1, 1,000:1, 500:1, 200:1, 100:1, 50:1, 20:1, 10:1, 5:1, 2:1, 1:1; 1:2; 1:5; 1:10; 1:100; 1:200; 1:500; 1:1,000; 1:2,000; 1:5,000; 1:10,000; 1:20,000; 1:50,000; 1:100,000; 1:500,000; 1:1,000,000; 1:2,000,000; 1:5,000,000; 1:10,000,000; 1:20,000,000; 1:50,000,000; or about 1:100,000,000, comparing numbers of total nucleated cells in each population. Cells in an isolated population of placental derived adherent cells can be combined with a plurality of cells of a plurality of cell types, as well.

In other embodiments, a population of the placental adherent cells described herein, e.g., the placental adherent cells described above, are combined with osteogenic placental adherent cells (OPACs), e.g., the OPACs described in patent application Ser. No. 12/546,556, filed Aug. 24, 2009, entitled “Methods and Compositions for Treatment of Bone Defects With Placental Stem Cells,” or combined with amnion-derived angiogenic cells (AMDACs), e.g., the AMDACs described in U.S. patent application Ser. No. 12/622,352, entitled “Amnion Derived Angiogenic Cells”, the disclosure of which is hereby incorporated by reference in its entirety.

### 5.6 Production of a Placental Cell Bank

Isolated cells from postpartum placentas, e.g., the isolated placental adherent cells described in Section 5.3, above, can be cultured in a number of different ways to produce a set of lots, e.g., wherein a lot is a set of individually-administrable doses, of isolated placental adherent cells. Such lots can, for example, be obtained from cells from placental perfusate or from cells from enzyme-digested placental tissue. Sets of lots of placental adherent cells, obtained from a plurality of placentas, can be arranged in a bank of isolated placental adherent cells for, e.g., long-term storage. Generally, tissue culture plastic-adherent placental adherent cells are obtained from an initial culture of placental material to form a seed culture, which is expanded under controlled conditions to form populations of cells from approximately equivalent numbers of doublings. Lots are preferably derived from the tissue of a single placenta, but can be derived from the tissue of a plurality of placentas.

In one embodiment, placental cell lots are obtained as follows. Placental tissue is first disrupted, e.g., by mincing, digested with a suitable enzyme, e.g., trypsin or collagenase (see Section 5.4.3, above). The placental tissue preferably comprises, e.g., the entire amnion, entire chorion, or both, from a single placenta, but can comprise only a part of either the amnion or chorion. The digested tissue is cultured, e.g., for about 1-3 weeks, preferably about 2 weeks. After removal of non-adherent cells, high-density colonies that form are collected, e.g., by trypsinization. These cells are collected and resuspended in a convenient volume of culture medium, and are then used to seed expansion cultures. Expansion cultures can be any arrangement of separate cell culture apparatuses, e.g., a Cell Factory by NUNC™. Cells can be subdivided to any degree so as to seed expansion cultures with, e.g., 1×10³, 2×10³, 3×10³, 4×10³, 5×10³, 6×10³, 7×10³, 8×10³, 9×10³, 1×10⁴, 1×10⁴, 2×10⁴, 3×10⁴, 4×10⁴, 5×10⁴, 6×10⁴, 7×10⁴, 8×10⁴, 9×10⁴, or 10×10⁴ cells/cm². Preferably, from about 1×10³ to about 1×10⁴ cells/cm² are used to seed each expansion culture. The number of expansion cultures may be greater or fewer in number depending upon the particular placenta(s) from which the cells are obtained.

Expansion cultures are grown until the density of cells in culture reaches a certain value, e.g., about 1×10⁵ cells/cm². Cells can either be collected and cryopreserved at this point, or passaged into new expansion cultures as described above. Cells can be passaged, e.g., 2, 3, 4, 5, 6, 7, 8, 9, 10, 11, 12, 13, 14, 15, 16, 17, 18, 19 or 20 times prior to use. A record of the cumulative number of population doublings is preferably maintained during expansion culture(s). The cells from a culture can be expanded for 2, 3, 4, 5, 6, 7, 8, 9, 10, 12, 14, 16, 18, 20, 22, 24, 26, 28, 30, 32, 34, 36, 38 or 40 doublings, or up to 60 doublings. Preferably, however, the number of population doublings, prior to dividing the population of cells into individual doses, is from about 15 to about 30. The cells can be culture continuously throughout the expansion process, or can be frozen at one or more points during expansion.

Cells to be used for individual doses can be frozen, e.g., cryopreserved for later use. Individual doses can comprise, e.g., about 1 million to about 50 million cells per ml, and can comprise between about 10⁶ and about 10¹⁰ cells in total.

In one embodiment, therefore, a placental cell bank can be made by a method comprising: expanding primary culture placental adherent cells from a human post-partum placenta for a first plurality of population doublings; cryopreserving said placental adherent cells to form a Master Cell Bank; expanding a plurality of placental adherent cells from the Master Cell Bank for a second plurality of population doublings; cryopreserving said placental adherent cells to form a Working Cell Bank; expanding a plurality of placental adherent cells from the Working Cell Bank for a third plurality of population doublings; and cryopreserving said placental adherent cells in individual doses, wherein said individual doses collectively compose a placental cell bank. Optionally, a plurality of placental adherent cells from said third plurality of population doublings can be expanded for a fourth plurality of population doublings and cryopreserved in individual doses, wherein said individual doses collectively compose a placental cell bank.

In another specific embodiment, said primary culture placental adherent cells comprise placental adherent cells from placental perfusate. In another specific embodiment, said primary culture placental adherent cells comprise placental adherent cells from digested placental tissue. In another specific embodiment, said primary culture placental adherent cells comprise placental adherent cells from placental perfusate and from digested placental tissue. In another specific embodiment, all of said placental adherent cells in said placental cell primary culture are from the same placenta. In another specific embodiment, the method further comprises the step of selecting CD200⁺ or HLA-G⁻ placental adherent cells from said plurality of said placental adherent cells from said Working Cell Bank to form individual doses. In another specific embodiment, said individual doses comprise from about 10⁴ to about 10⁵ placental adherent cells. In another specific embodiment, said individual doses comprise from about 10⁵ to about 10⁶ placental adherent cells. In another specific embodiment, said individual doses comprise from about 10⁶ to about 10⁷ placental adherent cells. In another specific embodiment, said individual doses comprise from about 10⁷ to about 10⁸ placental adherent cells. In another specific embodiment, said individual doses comprise from about 10⁸ to about 10⁹ placental adherent cells. In another specific embodiment, said individual doses comprise from about 10⁹ to about 10¹⁰ placental adherent cells.

In a preferred embodiment, the donor from which the placenta is obtained (e.g., the mother) is tested for at least one pathogen. If the mother tests positive for a tested pathogen, the entire lot from the placenta is discarded. Such testing can be performed at any time during production of placental cell lots, e.g., during expansion culture. Pathogens for which the presence is tested can include, without limitation, hepatitis A, hepatitis B, hepatitis C, hepatitis D, hepatitis E, human immunodeficiency virus (types I and II), cytomegalovirus, herpesvirus, and the like.

### 5.7 Preservation of Placental Cells

Isolated placental adherent cells, e.g., the isolated placental adherent cells described in Section 5.3, above, can be preserved, that is, placed under conditions that allow for long-term storage, or conditions that inhibit cell death by, e.g., apoptosis or necrosis.

Placental cells can be preserved using, e.g., a composition comprising an apoptosis inhibitor, necrosis inhibitor and/or an oxygen-carrying perfluorocarbon, as described in related U.S. Application Publication No. 2007/0190042, the disclosure of which is incorporated herein by reference in its entirety. In one embodiment, a method of preserving a population of cells, useful in the methods and compositions described herein, comprises contacting said population of cells with a cell collection composition comprising an inhibitor of apoptosis and an oxygen-carrying perfluorocarbon, wherein said inhibitor of apoptosis is present in an amount and for a time sufficient to reduce or prevent apoptosis in the population of cells, as compared to a population of cells not contacted with the inhibitor of apoptosis. In a specific embodiment, said inhibitor of apoptosis is a caspase inhibitor. In another specific embodiment, said inhibitor of apoptosis is a JNK inhibitor. In another specific embodiment, said JNK inhibitor does not modulate differentiation or proliferation of said cells. In another embodiment, said cell collection composition comprises said inhibitor of apoptosis and said oxygen-carrying perfluorocarbon in separate phases. In another embodiment, said cell collection composition comprises said inhibitor of apoptosis and said oxygen-carrying perfluorocarbon in an emulsion. In another embodiment, the cell collection composition additionally comprises an emulsifier, e.g., lecithin. In another embodiment, said apoptosis inhibitor and said perfluorocarbon are between about 0° C. and about 25° C. at the time of contacting the cells. In another specific embodiment, said apoptosis inhibitor and said perfluorocarbon are between about 2° C. and 10° C., or between about 2° C. and about 5° C., at the time of contacting the cells. In another specific embodiment, said contacting is performed during transport of said population of cells. In another specific embodiment, said contacting is performed during freezing and thawing of said population of cells.

Populations of placental adherent cells can be preserved, e.g., by a method comprising contacting said population of cells with an inhibitor of apoptosis and an organ-preserving compound, wherein said inhibitor of apoptosis is present in an amount and for a time sufficient to reduce or prevent apoptosis in the population of cells, as compared to a population of cells not contacted with the inhibitor of apoptosis. In a specific embodiment, the organ-preserving compound is UW solution (described in U.S. Pat. No. 4,798,824; also known as ViaSpan; see also Southard et al., Transplantation 49(2):251-257 (1990)) or a solution described in Stem et al., U.S. Pat. No. 5,552,267, the disclosures of which are hereby incorporated by reference in their entireties. In another embodiment, said organ-preserving compound is hydroxyethyl starch, lactobionic acid, raffinose, or a combination thereof. In another embodiment, the cell collection composition additionally comprises an oxygen-carrying perfluorocarbon, either in two phases or as an emulsion.

In another embodiment of the method, placental adherent cells are contacted with a cell collection composition comprising an apoptosis inhibitor and oxygen-carrying perfluorocarbon, organ-preserving compound, or combination thereof, during perfusion. In another embodiment, said cells are contacted during a process of tissue disruption, e.g., enzymatic digestion. In another embodiment, placental adherent cells are contacted with said cell collection compound after collection by perfusion, or after collection by tissue disruption, e.g., enzymatic digestion.

Typically, during placental cell collection, enrichment and isolation, it is preferable to minimize or eliminate cell stress due to hypoxia and mechanical stress. In another embodiment of the method, therefore, a cell, or population of cells, is exposed to a hypoxic condition during collection, enrichment or isolation for less than six hours during said preservation, wherein a hypoxic condition is a concentration of oxygen that is less than normal blood oxygen concentration. In another specific embodiment, said population of cells is exposed to said hypoxic condition for less than two hours during said preservation. In another specific embodiment, said population of cells is exposed to said hypoxic condition for less than one hour, or less than thirty minutes, or is not exposed to a hypoxic condition, during collection, enrichment or isolation. In another specific embodiment, said population of cells is not exposed to shear stress during collection, enrichment or isolation.

Placental cells can be cryopreserved, e.g., in cryopreservation medium in small containers, e.g., ampoules. Suitable cryopreservation medium includes, but is not limited to, culture medium including, e.g., growth medium, or cell freezing medium, for example commercially available cell freezing medium, e.g., C2695, C2639 or C6039 (Sigma). Cryopreservation medium preferably comprises DMSO (dimethylsulfoxide), at a concentration of about 2% to about 15% (v/v), e.g., about 10% (v/v). Cryopreservation medium may comprise additional agents, for example, methylcellulose and/or glycerol. Placental cells are preferably cooled at about 1° C./min during cryopreservation. A preferred cryopreservation temperature is about −80° C. to about −180° C., preferably about −125° C. to about −140° C. Cryopreserved cells can be transferred to liquid nitrogen prior to thawing for use. In some embodiments, for example, once the ampoules have reached about −90° C., they are transferred to a liquid nitrogen storage area. Cryopreservation can also be done using a controlled-rate freezer. Cryopreserved cells preferably are thawed at a temperature of about 25° C. to about 40° C., preferably to a temperature of about 37° C.

### 5.8 Compositions Comprising Isolated Placental Cells

The placental adherent cells described herein, e.g., at Section 5.3, can be combined with any physiologically-acceptable or medically-acceptable compound, composition or device for use in the methods and compositions described herein. Compositions useful in the methods of treatment provided herein can comprise any one or more of the placental adherent cells described herein (see Section 5.3, above). In certain embodiments, the composition is a pharmaceutically-acceptable composition, e.g., a composition comprising placental adherent cells in a pharmaceutically-acceptable carrier.

In certain embodiments, a composition comprising the isolated placental adherent cells additionally comprises a matrix, e.g., a decellularized matrix or a synthetic matrix. In another specific embodiment, said matrix is a three-dimensional scaffold. In another specific embodiment, said matrix comprises collagen, gelatin, laminin, fibronectin, pectin, ornithine, or vitronectin. In another ore specific embodiment, the matrix is an amniotic membrane or an amniotic membrane-derived biomaterial. In another specific embodiment, said matrix comprises an extracellular membrane protein. In another specific embodiment, said matrix comprises a synthetic compound. In another specific embodiment, said matrix comprises a bioactive compound. In another specific embodiment, said bioactive compound is a growth factor, cytokine, antibody, or organic molecule of less than 5,000 daltons.

In another embodiment, a composition useful in the methods of treatment provided herein comprises medium conditioned by any of the foregoing placental adherent cells, or any of the foregoing placental cell populations.

### 5.8.1 Cryopreserved Isolated Placental Cells

The isolated placental cell populations useful in the methods and compositions described herein can be preserved, for example, cryopreserved for later use. Methods for cryopreservation of cells, such as stem cells, are well known in the art. Isolated placental cell populations can be prepared in a form that is easily administrable to an individual, e.g., an isolated placental cell population that is contained within a container that is suitable for medical use. Such a container can be, for example, a syringe, sterile plastic bag, flask, jar, or other container from which the isolated placental cell population can be easily dispensed. For example, the container can be a blood bag or other plastic, medically-acceptable bag suitable for the intravenous administration of a liquid to a recipient. The container, in certain embodiments, is one that allows for cryopreservation of the combined cell population.

The cryopreserved isolated placental cell population can comprise isolated placental cell derived from a single donor, or from multiple donors. The isolated placental cell population can be completely HLA-matched to an intended recipient, or partially or completely HLA-mismatched.

Thus, in one embodiment, isolated placental adherent cells can be used in the methods and described herein in the form of a composition comprising a tissue culture plastic-adherent placental cell population in a container. In a specific embodiment, the isolated placental adherent cells are cryopreserved. In another specific embodiment, the container is a bag, flask, or jar. In another specific embodiment, said bag is a sterile plastic bag. In another specific embodiment, said bag is suitable for, allows or facilitates intravenous administration of said isolated placental cell population, e.g., by intravenous infusion. The bag can comprise multiple lumens or compartments that are interconnected to allow mixing of the isolated placental adherent cells and one or more other solutions, e.g., a drug, prior to, or during, administration. In another specific embodiment, the composition comprises one or more compounds that facilitate cryopreservation of the combined cell population. In another specific embodiment, said isolated placental cell population is contained within a physiologically-acceptable aqueous solution. In another specific embodiment, said physiologically-acceptable aqueous solution is a 0.9% NaCl solution. In another specific embodiment, said isolated placental cell population comprises placental adherent cells that are HLA-matched to a recipient of said cell population. In another specific embodiment, said combined cell population comprises placental adherent cells that are at least partially HLA-mismatched to a recipient of said cell population. In another specific embodiment, said isolated placental adherent cells are derived from a plurality of donors.

In certain embodiments, the isolated placental adherent cells in the container are isolated CD10⁺, CD34⁻, CD105⁺ placental adherent cells, wherein said cells have been cryopreserved, and are contained within a container. In a specific embodiment, said CD10⁺, CD34⁻, CD105⁺ placental adherent cells are also CD200⁺. In another specific embodiment, said CD10⁺, CD34⁻, CD105⁺, CD200⁺ placental adherent cells are also CD45⁻ or CD90⁺. In another specific embodiment, said CD10⁺, CD34⁻, CD105⁺, CD200⁺ placental adherent cells are also CD45⁻ and CD90⁺. In another specific embodiment, the CD34⁻, CD10⁺, CD105⁺ placental adherent cells are additionally one or more of CD13⁺, CD29⁺, CD33⁺, CD38⁻, CD44⁺, CD45⁻, CD54⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁺), CD106/VCAM⁺, CD117⁻, CD144/VE-cadherin^{dim}, CD184/CXCR4⁻, CD200⁺, CD133⁻, OCT-4⁺, SSEA4⁻, SSEA4⁻, ABC-p⁺, KDR⁻ (VEGFR2⁻), HLA-A,B,C⁺, HLA-DP,DQ,DR⁻, HLA-G⁻, or Programmed Death-1 Ligand (PDL1)⁺, or any combination thereof. In another specific embodiment, the CD34⁻, CD10⁺, CD105⁺ placental adherent cells are additionally CD13⁺, CD29⁺, CD33⁺, CD38⁻, CD44⁺, CD45⁻, CD54/ICAM⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁺), CD106/VCAM⁺, CD117⁻, CD144/VE-cadherin^{dim}, CD184/CXCR4⁺, CD200⁺, CD133⁻, OCT-4⁺, SSEA3⁻, SSEA4⁻, ABC-p⁺, KDR⁻ (VEGFR2⁻), HLA-A,B,C⁺, HLA-DP,DQ,DR⁻, HLA-G⁻, and Programmed Death-1 Ligand (PDL1)⁺.

In certain other embodiments, the above-referenced isolated placental adherent cells are isolated CD200⁺, HLA-G⁻ placental adherent cells, wherein said cells have been cryopreserved, and are contained within a container. In another embodiment, the isolated placental adherent cells are CD73⁺, CD105⁺, CD200⁺ cells that have been cryopreserved, and are contained within a container. In another embodiment, the isolated placental adherent cells are CD200⁺, OCT-4⁺ stem cells that have been cryopreserved, and are contained within a container. In another embodiment, the isolated placental adherent cells are CD73⁺, CD105⁺ cells that have been cryopreserved, and are contained within a container, and wherein said isolated placental adherent cells facilitate the formation of one or more embryoid-like bodies when cultured with a population of placental adherent cells under conditions that allow for the formation of embryoid-like bodies. In another embodiment, the isolated placental adherent cells are CD73⁺, CD105⁺, HLA-G⁻ cells that have been cryopreserved, and are contained within a container. In another embodiment, the isolated placental adherent cells are OCT-4⁺ placental adherent cells that have been cryopreserved, and are contained within a container, and wherein said cells facilitate the formation of one or more embryoid-like bodies when cultured with a population of placental adherent cells under conditions that allow for the formation of embryoid-like bodies.

In another specific embodiment, the above-referenced isolated placental adherent cells are placental stem cells or placental multipotent cells that are CD34⁻, CD10⁺ and CD105⁺ as detectable by flow cytometry (e.g., placental adherent cells). In another specific embodiment, the isolated CD34⁻, CD10⁺, CD105⁺ placental adherent cells have the potential to differentiate into cells of a neural phenotype, cells of an osteogenic phenotype, or cells of a chondrogenic phenotype. In another specific embodiment, the isolated CD34⁻, CD10⁺, CD105⁺ placental adherent cells are additionally CD200⁺. In another specific embodiment, the isolated CD34⁻, CD10⁺, CD105⁺ placental adherent cells are additionally CD90⁺ or CD45⁻, as detectable by flow cytometry. In another specific embodiment, the isolated CD34⁻, CD10⁺, CD105⁺ placental adherent cells are additionally CD90⁺ or CD45⁻, as detectable by flow cytometry. In another specific embodiment, the CD34⁻, CD10⁺, CD105⁺, CD200⁺ placental adherent cells are additionally CD90⁺ or CD45⁻, as detectable by flow cytometry. In another specific embodiment, the CD34⁻, CD10⁺, CD105⁺, CD200⁺ cells are additionally CD90⁺ and CD45⁻, as detectable by flow cytometry. In another specific embodiment, the CD34⁻, CD10⁺, CD105⁺, CD200⁺, CD90⁺, CD45⁻ cells are additionally CD80⁻ and CD86⁻, as detectable by flow cytometry. In another specific embodiment, the CD34⁻, CD10⁺, CD105⁺ cells are additionally one or more of CD29⁺, CD38⁻, CD44⁺, CD54⁺, CD80⁻, CD86⁻, SH3⁺ or SH4⁺. In another specific embodiment, the cells are additionally CD44⁺. In a specific embodiment of any of the isolated CD34⁻, CD10⁺, CD105⁺ placental adherent cells above, the cells are additionally one or more of CD117⁻, CD133⁻, KDR⁻ (VEGFR2⁻), HLA-A,B,C⁺, HLA-DP,DQ,DR⁻, and/or PDL1⁺.

In a specific embodiment of any of the foregoing cryopreserved isolated placental adherent cells, said container is a bag. In various specific embodiments, said container comprises about, at least, or at most 1×10⁶ said isolated placental adherent cells, 5×10⁶ said isolated placental adherent cells, 1×10⁷ said isolated placental adherent cells, 5×10⁷ said isolated placental adherent cells, 1×10⁸ said isolated placental adherent cells, 5×10⁸ said isolated placental adherent cells, 1×10⁹ said isolated placental adherent cells, 5×10⁹ said isolated placental adherent cells, 1×10¹⁰ said isolated placental adherent cells, or 5×10¹⁰ said isolated placental adherent cells. In other specific embodiments of any of the foregoing cryopreserved populations, said isolated placental adherent cells have been passaged about, at least, or no more than 5 times, no more than 10 times, no more than 15 times, or no more than 20 times. In another specific embodiment of any of the foregoing cryopreserved isolated placental adherent cells, said isolated placental adherent cells have been expanded within said container.

In certain embodiments, a single unit dose of placental derived adherent cells can comprise, in various embodiments, about, at least, or no more than 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more placental derived adherent cells. In certain embodiments, the pharmaceutical compositions provided herein comprises populations of placental derived adherent cells, that comprise 50% viable cells or more (that is, at least 50% of the cells in the population are functional or living). Preferably, at least 60% of the cells in the population are viable. More preferably, at least 70%, 80%, 90%, 95%, or 99% of the cells in the population in the pharmaceutical composition are viable.

### 5.8.2 Genetically Engineered Placental Cells

Further provided herein are placental adherent cells, e.g., any of the placental multipotent cells or placental adherent cells described in Section 5.3, above, or pharmaceutical compositions comprising such placental adherent cells, wherein the placental adherent cells have been genetically engineered to produce recombinant or exogenous cytokines associated with, or which promote, angiogenesis. In certain embodiments, said proteins that facilitate angiogenesis are one or more of hepatocyte growth factor (HGF), vascular endothelial growth factor (VEGF) (e.g., VEGFD), fibroblast growth factor (FGF) (e.g., FGF2), angiogenin (ANG), epidermal growth factor (EGF), epithelial-neutrophil-activating protein 78 (ENA-78), follistatin, granulocyte colony-stimulating factor (G-CSF), growth-regulated ongogene protein (GRO), interleukin-6 (IL-6), IL-8, leptin, monocyte chemotactic protein-1 (MCP-1), MCP-3, platelet-derived growth factor submunit B (PDGFB), rantes, transforming growth factor beta 1 (TGF-01), thrombopoitein (Tpo), tissue inhibitor of metalloproteinases 1 (TIMP1), TIMP2, and/or urokinase plasminogen activator receptor (uPAR).

Methods for genetically engineering cells, for example with retroviral vectors, adenoviral vectors, adeno-associated viral vectors, polyethylene glycol, or other methods known to those skilled in the art, can be used. These include using expression vectors which transport and express nucleic acid molecules in the cells. (See Goeddel; Gene Expression Technology: Methods in Enzymology 185, Academic Press, San Diego, Calif. (1990)). Vector DNA can be introduced into prokaryotic or eukaryotic cells via conventional transformation or transfection techniques. Suitable methods for transforming or transfecting host cells can be found in Sambrook et al. Molecular Cloning: A Laboratory Manual, 2nd Edition, Cold Spring Harbor Laboratory press (1989), and other laboratory textbooks.

Placental cells, e.g., the placental adherent cells described in Section 5.3, above, can be genetically modified by introducing DNA or RNA into the cell, e.g., DNA or RNA encoding a protein of interest, by methods including viral transfer, including the use of DNA or RNA viral vectors, such as retroviruses (including lentiviruses), Simian virus 40 (SV40), adenovirus, Sindbis virus, and bovine papillomavirus for example; chemical transfer, including calcium phosphate transfection and DEAE dextran transfection methods; membrane fusion transfer, using DNA-loaded membrane vesicles such as liposomes, red blood cell ghosts, and protoplasts, for example; or physical transfer techniques, such as microinjection, electroporation, or naked DNA transfer. The placental adherent cells can be genetically altered by insertion of exogenous DNA, or by substitution of a segment of the cellular genome with exogenous DNA. Insertion of exogenous DNA sequence(s) can be accomplished, e.g., by homologous recombination or by viral integration into the host cell genome, or by incorporating the DNA into the cell, particularly into its nucleus, using a plasmid expression vector and a nuclear localization sequence. The DNA can comprise one or more promoters that allow positive or negative induction of expression of the protein of interest using certain chemicals/drugs, e.g., tetracycline; the promoters can, in other embodiments, be constitutive.

Calcium phosphate transfection can be used to introduce, e.g., plasmid DNA containing a polynucleotide sequence encoding the protein of interest, into a cell. In certain embodiments, DNA is combined with a solution of calcium chloride, then added to a phosphate-buffered solution. Once a precipitate has formed, the solution is added directly to cultured cells. Treatment with DMSO or glycerol can be used to improve transfection efficiency, and levels of stable transfectants can be improved using bis-hydroxyethylamino ethanesulfonate (BES). Calcium phosphate transfection systems are commercially available (e.g., PROFECTION®, Promega Corp., Madison, Wis.). DEAE-dextran transfection may also be used.

Isolated placental adherent cells may also be genetically engineered by microinjection. In certain embodiments, a glass micropipette is guided into the nucleus of cells under a light microscope to inject DNA or RNA.

Placental cells can also be genetically modified using electroporation. In certain embodiments, DNA or RNA is added to a suspension of cultured cells, and the DNA/RNA-cell suspension is placed between two electrodes and subjected to an electrical pulse, causing a transient permeability in the cell's outer membrane that is manifested by the appearance of pores across the membrane.

Liposomal delivery of DNA or RNA to genetically modify the cells can be performed using cationic liposomes, optionally including dioleoyl phosphatidylethanolamine (DOPE) or dioleoyl phosphatidylcholine (DOPC), e.g., LIPOFECTIN® (Life Technologies, Inc.). Other commercially-available delivery systems include EFFECTENE™ (Qiagen), DOTAP (Roche Molecular Biochemicals), FUGENE 6™ (Roche Molecular Biochemicals), and TRANSFECTAM® (Promega).

Viral vectors can be used to genetically alter placental adherent cells by delivery of, e.g., target genes, polynucleotides, antisense molecules, or ribozyme sequences into the cells. Retroviral vectors are effective for transducing rapidly-dividing cells, although a number of retroviral vectors have been developed to effectively transfer DNA into non-dividing cells as well. Packaging cell lines for retroviral vectors are known to those of skill in the art. In certain embodiments, a retroviral DNA vector contains two retroviral LTRs such that a first LTR is located 5′ to the SV40 promoter, which is operationally linked to the target gene sequence cloned into a multicloning site, followed by a 3′ second LTR. Once formed, the retroviral DNA vector is transferred into a packaging cell line using calcium phosphate-mediated transfection, as previously described. Following approximately 48 hours of virus production, the viral vector, now containing the target gene sequence, is harvested. Methods of transfecting cells using lentiviral vectors, recombinant herpes viruses, adenoviral vectors, or alphavirus vectors are known in the art.

Successful transfection or transduction of target cells can be demonstrated using genetic markers, in a technique that is known to those of skill in the art. The green fluorescent protein of Aequorea victoria, for example, has been shown to be an effective marker for identifying and tracking genetically modified hematopoietic cells. Alternative selectable markers include the R-Gal gene, truncated nerve growth factor receptor, or drug selectable markers (including but not limited to NEO, MTX, or hygromycin).

### 5.8.3 Pharmaceutical Compositions

Populations of isolated placental adherent cells, e.g., placental adherent cells, or populations of cells comprising the isolated placental adherent cells, can be formulated into pharmaceutical compositions for use in vivo, e.g., in the methods of treatment provided herein. Such pharmaceutical compositions comprise a population of isolated placental adherent cells, or a population of cells comprising isolated placental adherent cells, in a pharmaceutically-acceptable carrier, e.g., a saline solution or other accepted physiologically-acceptable solution for in vivo administration. Pharmaceutical compositions comprising the isolated placental adherent cells described herein can comprise any, or any combination, of the isolated placental cell populations, or isolated placental adherent cells, described elsewhere herein. The pharmaceutical compositions can comprise fetal, maternal, or both fetal and maternal isolated placental adherent cells. The pharmaceutical compositions provided herein can further comprise isolated placental adherent cells obtained from a single individual or placenta, or from a plurality of individuals or placentae.

The pharmaceutical compositions provided herein can comprise any number of isolated placental adherent cells. For example, a single unit dose of isolated placental adherent cells can comprise, in various embodiments, about, at least, or no more than 1×10×5, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more isolated placental adherent cells.

The pharmaceutical compositions provided herein comprise populations of cells that comprise 50% viable cells or more (that is, at least 50% of the cells in the population are functional or living). Preferably, at least 60% of the cells in the population are viable. More preferably, at least 70%, 80%, 90%, 95%, or 99% of the cells in the population in the pharmaceutical composition are viable.

The pharmaceutical compositions provided herein can comprise one or more compounds that, e.g., facilitate engraftment (e.g., anti-T-cell receptor antibodies, an immunosuppressant, or the like); stabilizers such as albumin, dextran 40, gelatin, hydroxyethyl starch, plasmalyte, and the like.

When formulated as an injectable solution, in one embodiment, the pharmaceutical composition comprises about 1% to 1.5% HSA and about 2.5% dextran. In a preferred embodiment, the pharmaceutical composition comprises from about 5×10⁶ cells per milliliter to about 2×10⁷ cells per milliliter in a solution comprising 5% HSA and 10% dextran, optionally comprising an immunosuppressant, e.g., cyclosporine A at, e.g., 10 mg/kg.

In other embodiments, the pharmaceutical composition, e.g., a solution, comprises a plurality of cells, e.g., isolated placental adherent cells, for example, placental stem cells or placental multipotent cells, wherein said pharmaceutical composition comprises between about 1.0±0.3×10⁶ cells per milliliter to about 5.0±1.5×10⁶ cells per milliliter. In other embodiments, the pharmaceutical composition comprises between about 1.5×10⁶ cells per milliliter to about 3.75×10⁶ cells per milliliter. In other embodiments, the pharmaceutical composition comprises between about 1×10⁶ cells/mL to about 50×10⁶ cells/mL, about 1×10⁶ cells/mL to about 40×10⁶ cells/mL, about 1×10⁶ cells/mL to about 30×10⁶ cells/mL, about 1×10⁶ cells/mL to about 20×10⁶ cells/mL, about 1×10⁶ cells/mL to about 15×10⁶ cells/mL, or about 1×10⁶ cells/mL to about 10×10⁶ cells/mL. In certain embodiments, the pharmaceutical composition comprises no visible cell clumps (i.e., no macro cell clumps), or substantially no such visible clumps. As used herein, “macro cell clumps” means an aggregation of cells visible without magnification, e.g., visible to the naked eye, and generally refers to a cell aggregation larger than about 150 microns In some embodiments, the pharmaceutical composition comprises about 2.5%, 3.0%, 3.5%, 4.0%, 4.5%, 5.0%, 5.5%, 6.0%, 6.5%, 7.0%, 7.5% 8.0%, 8.5%, 9.0%, 9.5% or 10% dextran, e.g., dextran-40. In a specific embodiment, said composition comprises about 7.5% to about 9% dextran-40. In a specific embodiment, said composition comprises about 5.5% dextran-40. In certain embodiments, the pharmaceutical composition comprises from about 1% to about 15% human serum albumin (HSA). In specific embodiments, the pharmaceutical composition comprises about 1%, 2%, 3%, 4%, 5%, 65, 75, 8%, 9%, 10%, 11%, 12%, 13%, 14% or 15% HSA. In a specific embodiment, said cells have been cryopreserved and thawed. In another specific embodiment, said cells have been filtered through a 70 μM to 100 μM filter. In another specific embodiment, said composition comprises no visible cell clumps. In another specific embodiment, said composition comprises fewer than about 200 cell clumps per 10⁶ cells, wherein said cell clumps are visible only under a microscope, e.g., a light microscope. In another specific embodiment, said composition comprises fewer than about 150 cell clumps per 10⁶ cells, wherein said cell clumps are visible only under a microscope, e.g., a light microscope. In another specific embodiment, said composition comprises fewer than about 100 cell clumps per 10⁶ cells, wherein said cell clumps are visible only under a microscope, e.g., a light microscope.

In a specific embodiment, the pharmaceutical composition comprises about 1.0±0.3×10⁶ cells per milliliter, about 5.5% dextran-40 (w/v), about 10% HSA (w/v), and about 5% DMSO (v/v).

In other embodiments, the pharmaceutical composition comprises a plurality of cells, e.g., a plurality of isolated placental adherent cells in a solution comprising 10% dextran-40, wherein the pharmaceutical composition comprises between about ±0.3×10⁶ cells per milliliter to about 5.0±1.5×10⁶ cells per milliliter, and wherein said composition comprises no cell clumps visible with the unaided eye (i.e., comprises no macro cell clumps). In some embodiments, the pharmaceutical composition comprises between about 1.5×10⁶ cells per milliliter to about 3.75×10⁶ cells per milliliter. In a specific embodiment, said cells have been cryopreserved and thawed. In another specific embodiment, said cells have been filtered through a 70 μm to 100 μm filter. In another specific embodiment, said composition comprises fewer than about 200 micro cell clumps (that is, cell clumps visible only with magnification) per 10⁶ cells. In another specific embodiment, the pharmaceutical composition comprises fewer than about 150 micro cell clumps per 10⁶ cells. In another specific embodiment, the pharmaceutical composition comprises fewer than about 100 micro cell clumps per 10⁶ cells. In another specific embodiment, the pharmaceutical composition comprises less than 15%, 14%, 13%, 12%, 11%, 10%, 9%, 8%, 7%, 6%, 5%, 4%, 3% or 2% DMSO, or less than 1%, 0.9%, 0.8%, 0.7%, 0.6%, 0.5%, 0.4%, 0.3%, 0.2%, or 0.1% DMSO.

Further provided herein are compositions comprising cells, wherein said compositions are produced by one of the methods disclosed herein. For example, in one embodiment, the pharmaceutical composition comprises cells, wherein the pharmaceutical composition is produced by a method comprising filtering a solution comprising placental adherent cells, e.g., placental stem cells or placental multipotent cells, to form a filtered cell-containing solution; diluting the filtered cell-containing solution with a first solution to about 1 to 50×10⁶, 1 to 40×10⁶, 1 to 30×10⁶, 1 to 20×10⁶, 1 to 15×10⁶, or 1 to 10×10⁶ cells per milliliter, e.g., prior to cryopreservation; and diluting the resulting filtered cell-containing solution with a second solution comprising dextran, but not comprising human serum albumin (HSA) to produce said composition. In certain embodiments, said diluting is to no more than about 15×10⁶ cells per milliliter. In certain embodiments, said diluting is to no more than about 10±3×10⁶ cells per milliliter. In certain embodiments, said diluting is to no more than about 7.5×10⁶ cells per milliliter. In other certain embodiments, if the filtered cell-containing solution, prior to the dilution, comprises less than about 15×10⁶ cells per milliliter, filtration is optional. In other certain embodiments, if the filtered cell-containing solution, prior to the dilution, comprises less than about 10±3×10⁶ cells per milliliter, filtration is optional. In other certain embodiments, if the filtered cell-containing solution, prior to the dilution, comprises less than about 7.5×10⁶ cells per milliliter, filtration is optional.

In a specific embodiment, the cells are cryopreserved between said diluting with a first dilution solution and said diluting with said second dilution solution. In another specific embodiment, the first dilution solution comprises dextran and HSA. The dextran in the first dilution solution or second dilution solution can be dextran of any molecular weight, e.g., dextran having a molecular weight of from about 10 kDa to about 150 kDa. In some embodiments, said dextran in said first dilution solution or said second solution is about 2.5%, 3.0%, 3.5%, 4.0%, 4.5%, 5.0%, 5.5%, 6.0%, 6.5%, 7.0%, 7.5% 8.0%, 8.5%, 9.0%, 9.5% or 10% dextran. In another specific embodiment, the dextran in said first dilution solution or said second dilution solution is dextran-40. In another specific embodiment, the dextran in said first dilution solution and said second dilution solution is dextran-40. In another specific embodiment, said dextran-40 in said first dilution solution is 5.0% dextran-40. In another specific embodiment, said dextran-40 in said first dilution solution is 5.5% dextran-40. In another specific embodiment, said dextran-40 in said second dilution solution is 10% dextran-40. In another specific embodiment, said HSA in said solution comprising HSA is 1 to 15% HSA. In another specific embodiment, said HSA in said solution comprising HSA is about 1%, 2%, 3%, 4%, 5%, 6%, 7%, 8%, 9%, 10%, 11%, 12%, 13%, 14% or 15% HSA. In another specific embodiment, said HSA in said solution comprising HSA is 10% HSA. In another specific embodiment, said first dilution solution comprises HSA. In another specific embodiment, said HSA in said first dilution solution is 10% HSA. In another specific embodiment, said first dilution solution comprises a cryoprotectant. In another specific embodiment, said cryoprotectant is DMSO. In another specific embodiment, said dextran-40 in said second dilution solution is about 10% dextran-40. In another specific embodiment, said composition comprising cells comprises about 7.5% to about 9% dextran. In another specific embodiment, the pharmaceutical composition comprises from about 1.0±0.3×10⁶ cells per milliliter to about 5.0±1.5×10⁶ cells per milliliter. In another specific embodiment, the pharmaceutical composition comprises from about 1.5×10⁶ cells per milliliter to about 3.75×10⁶ cells per milliliter.

In another embodiment, the pharmaceutical composition is made by a method comprising (a) filtering a cell-containing solution comprising placental adherent cells, e.g., placental stem cells or placental multipotent cells, prior to cryopreservation to produce a filtered cell-containing solution; (b) cryopreserving the cells in the filtered cell-containing solution at about 1 to 50×10⁶, 1 to 40×10⁶, 1 to 30×10⁶, 1 to 20×10⁶, 1 to 15×10⁶, or 1 to 10×10⁶ cells per milliliter; (c) thawing the cells; and (d) diluting the filtered cell-containing solution about 1:1 to about 1:11 (v/v) with a dextran-40 solution. In certain embodiments, if the number of cells is less than about 10±3×10⁶ cells per milliliter prior to step (a), filtration is optional. In another specific embodiment, the cells in step (b) are cryopreserved at about 10±3×10⁶ cells per milliliter. In another specific embodiment, the cells in step (b) are cryopreserved in a solution comprising about 5% to about 10% dextran-40 and HSA. In certain embodiments, said diluting in step (b) is to no more than about 15×10⁶ cells per milliliter.

In another embodiment, the pharmaceutical composition is made by a method comprising: (a) suspending placental adherent cells, e.g., placental stem cells or placental multipotent cells, in a 5.5% dextran-40 solution that comprises 10% HSA to form a cell-containing solution; (b) filtering the cell-containing solution through a 70 μm filter; (c) diluting the cell-containing solution with a solution comprising 5.5% dextran-40, 10% HSA, and 5% DMSO to about 1 to 50×10⁶, 1 to 40×10⁶, 1 to 30×10⁶, 1 to 20×10⁶, 1 to 15×10⁶, or 1 to 10×10⁶ cells per milliliter; (d) cryopreserving the cells; (e) thawing the cells; and (f) diluting the cell-containing solution 1:1 to 1:11 (v/v) with 10% dextran-40. In certain embodiments, said diluting in step (c) is to no more than about 15×10⁶ cells per milliliter. In certain embodiments, said diluting in step (c) is to no more than about 10±3×10⁶ cells/mL. In certain embodiments, said diluting in step (c) is to no more than about 7.5×10⁶ cells/mL.

In another embodiment, the composition comprising cells is made by a method comprising: (a) centrifuging a plurality of cells to collect the cells; (b) resuspending the cells in 5.5% dextran-40; (c) centrifuging the cells to collect the cells; (d) resuspending the cells in a 5.5% dextran-40 solution that comprises 10% HSA; (e) filtering the cells through a 70 μm filter; (f) diluting the cells in 5.5% dextran-40, 10% HSA, and 5% DMSO to about 1 to 50×10⁶, 1 to 40×10⁶, 1 to 30×10⁶, 1 to 20×10⁶, 1 to 15×10⁶, or 1 to 10×10⁶ cells per milliliter; (g) cryopreserving the cells; (h) thawing the cells; and (i) diluting the cells 1:1 to 1:11 (v/v) with 10% dextran-40. In certain embodiments, said diluting in step (f) is to no more than about 15×10⁶ cells per milliliter. In certain embodiments, said diluting in step (f) is to no more than about 10±3×10⁶ cells/mL. In certain embodiments, said diluting in step (f) is to no more than about 7.5×10⁶ cells/mL. In other certain embodiments, if the number of cells is less than about 10±3×10⁶ cells per milliliter, filtration is optional.

The compositions, e.g., pharmaceutical compositions comprising the isolated placental adherent cells, described herein can comprise any of the isolated placental adherent cells described herein.

Other injectable formulations, suitable for the administration of cellular products, may be used.

In one embodiment, the pharmaceutical composition comprises isolated placental adherent cells that are substantially, or completely, non-maternal in origin, that is, have the fetal genotype; e.g., at least about 90%, 95%, 98%, 99% or about 100% are non-maternal in origin. For example, in one embodiment a pharmaceutical composition comprises a population of isolated placental adherent cells that are CD200⁺ and HLA-G⁻; CD73⁺, CD105⁺, and CD200⁺; CD200⁺ and OCT-4⁺; CD73⁺, CD105⁺ and HLA-G⁻; CD73⁺ and CD105⁺ and facilitate the formation of one or more embryoid-like bodies in a population of placental adherent cells comprising said population of isolated placental cell when said population of placental adherent cells is cultured under conditions that allow the formation of an embryoid-like body; or OCT-4⁺ and facilitate the formation of one or more embryoid-like bodies in a population of placental adherent cells comprising said population of isolated placental cell when said population of placental adherent cells is cultured under conditions that allow the formation of an embryoid-like body; or a combination of the foregoing, wherein at least 70%, 80%, 90%, 95% or 99% of said isolated placental adherent cells are non-maternal in origin. In another embodiment, a pharmaceutical composition comprises a population of isolated placental adherent cells that are CD10⁺, CD105⁺ and CD34⁻; CD10⁺, CD105⁺, CD200⁺ and CD34⁻; CD10⁺, CD105⁺, CD200⁺, CD34⁻ and at least one of CD90⁺ or CD45⁻; CD10⁺, CD90⁺, CD105⁺, CD200⁺, CD34⁻ and CD45⁻; CD10⁺, CD90⁺, CD105⁺, CD200⁺, CD34⁻ and CD45⁻; CD200⁺ and HLA-G⁻; CD73⁺, CD105⁺, and CD200⁺; CD200⁺ and OCT-4⁺; CD73⁺, CD105⁺ and HLA-G⁻; CD73⁺ and CD105⁺ and facilitate the formation of one or more embryoid-like bodies in a population of placental adherent cells comprising said isolated placental adherent cells when said population of placental adherent cells is cultured under conditions that allow the formation of an embryoid-like body; OCT-4⁺ and facilitate the formation of one or more embryoid-like bodies in a population of placental adherent cells comprising said isolated placental adherent cells when said population of placental adherent cells is cultured under conditions that allow the formation of an embryoid-like body; or one or more of CD117⁻, CD133⁻, KDR⁻, CD80⁻, CD86⁻, HLA-A,B,C⁺, HLA-DP,DQ,DR⁻ and/or PDL1⁺; or a combination of the foregoing, wherein at least 70%, 80%, 90%, 95% or 99% of said isolated placental adherent cells are non-maternal in origin. In a specific embodiment, the pharmaceutical composition additionally comprises a stem cell that is not obtained from a placenta.

Isolated placental adherent cells in the compositions, e.g., pharmaceutical compositions, provided herein, can comprise placental adherent cells derived from a single donor, or from multiple donors. The isolated placental adherent cells can be completely HLA-matched to an intended recipient, or partially or completely HLA-mismatched.

### 5.8.4 Matrices Comprising Isolated Placental Cells

Further provided herein are compositions comprising matrices, hydrogels, scaffolds, and the like that comprise a placental cell, or a population of isolated placental adherent cells. Such compositions can be used in the place of, or in addition to, cells in liquid suspension.

The isolated placental adherent cells described herein can be seeded onto a natural matrix, e.g., a placental biomaterial such as an amniotic membrane material. Such an amniotic membrane material can be, e.g., amniotic membrane dissected directly from a mammalian placenta; fixed or heat-treated amniotic membrane, substantially dry (i.e., <20% H₂O) amniotic membrane, chorionic membrane, substantially dry chorionic membrane, substantially dry amniotic and chorionic membrane, and the like. Preferred placental biomaterials on which isolated placental adherent cells can be seeded are described in Hariri, U.S. Application Publication No. 2004/0048796, the disclosure of which is incorporated herein by reference in its entirety.

The isolated placental adherent cells described herein can be suspended in a hydrogel solution suitable for, e.g., injection. Suitable hydrogels for such compositions include self-assembling peptides, such as RAD16. In one embodiment, a hydrogel solution comprising the cells can be allowed to harden, for instance in a mold, to form a matrix having cells dispersed therein for implantation. Isolated placental adherent cells in such a matrix can also be cultured so that the cells are mitotically expanded prior to implantation. The hydrogel is, e.g., an organic polymer (natural or synthetic) that is cross-linked via covalent, ionic, or hydrogen bonds to create a three-dimensional open-lattice structure that entraps water molecules to form a gel. Hydrogel-forming materials include polysaccharides such as alginate and salts thereof, peptides, polyphosphazines, and polyacrylates, which are crosslinked ionically, or block polymers such as polyethylene oxide-polypropylene glycol block copolymers which are crosslinked by temperature or pH, respectively. In some embodiments, the hydrogel or matrix is biodegradable.

In some embodiments, the formulation comprises an in situ polymerizable gel (see, e.g., U.S. Patent Application Publication 2002/0022676, the disclosure of which is incorporated herein by reference in its entirety; Anseth et al., J. Control Release, 78(1-3):199-209 (2002); Wang et al., Biomaterials, 24(22):3969-80 (2003).

In some embodiments, the polymers are at least partially soluble in aqueous solutions, such as water, buffered salt solutions, or aqueous alcohol solutions, that have charged side groups, or a monovalent ionic salt thereof. Examples of polymers having acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

In a specific embodiment, the matrix is a felt, which can be composed of a multifilament yarn made from a bioabsorbable material, e.g., PGA, PLA, PCL copolymers or blends, or hyaluronic acid. The yarn is made into a felt using standard textile processing techniques consisting of crimping, cutting, carding and needling. In another preferred embodiment the cells of the invention are seeded onto foam scaffolds that may be composite structures. In addition, the three-dimensional framework may be molded into a useful shape, such as a specific structure in the body to be repaired, replaced, or augmented. Other examples of scaffolds that can be used include nonwoven mats, porous foams, or self assembling peptides. Nonwoven mats can be formed using fibers comprised of a synthetic absorbable copolymer of glycolic and lactic acids (e.g., PGA/PLA) (VICRYL, Ethicon, Inc., Somerville, N.J.). Foams, composed of, e.g., poly(.epsilon.-caprolactone)/poly(glycolic acid) (PCL/PGA) copolymer, formed by processes such as freeze-drying, or lyophilization (see, e.g., U.S. Pat. No. 6,355,699), can also be used as scaffolds.

The isolated placental adherent cells described herein or co-cultures thereof can be seeded onto a three-dimensional framework or scaffold and implanted in vivo. Such a framework can be implanted in combination with any one or more growth factors, cells, drugs or other components that, e.g., stimulate tissue formation.

Examples of scaffolds that can be used include nonwoven mats, porous foams, or self assembling peptides. Nonwoven mats can be formed using fibers comprised of a synthetic absorbable copolymer of glycolic and lactic acids (e.g., PGA/PLA) (VICRYL, Ethicon, Inc., Somerville, N.J.). Foams, composed of, e.g., poly(ε-caprolactone)/poly(glycolic acid) (PCL/PGA) copolymer, formed by processes such as freeze-drying, or lyophilization (see, e.g., U.S. Pat. No. 6,355,699), can also be used as scaffolds.

In another embodiment, isolated placental adherent cells can be seeded onto, or contacted with, a felt, which can be, e.g., composed of a multifilament yarn made from a bioabsorbable material such as PGA, PLA, PCL copolymers or blends, or hyaluronic acid.

The isolated placental adherent cells provided herein can, in another embodiment, be seeded onto foam scaffolds that may be composite structures. Such foam scaffolds can be molded into a useful shape, such as that of a portion of a specific structure in the body to be repaired, replaced or augmented. In some embodiments, the framework is treated, e.g., with 0.1M acetic acid followed by incubation in polylysine, PBS, and/or collagen, prior to inoculation of the cells in order to enhance cell attachment. External surfaces of a matrix may be modified to improve the attachment or growth of cells and differentiation of tissue, such as by plasma-coating the matrix, or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate, etc.), a cellular matrix, and/or other materials such as, but not limited to, gelatin, alginates, agar, agarose, and plant gums, and the like.

In some embodiments, the scaffold comprises, or is treated with, materials that render it non-thrombogenic. These treatments and materials may also promote and sustain endothelial growth, migration, and extracellular matrix deposition. Examples of these materials and treatments include but are not limited to natural materials such as basement membrane proteins such as laminin and Type IV collagen, synthetic materials such as EPTFE, and segmented polyurethaneurea silicones, such as PURSPAN™ (The Polymer Technology Group, Inc., Berkeley, Calif.). The scaffold can also comprise anti-thrombotic agents such as heparin; the scaffolds can also be treated to alter the surface charge (e.g., coating with plasma) prior to seeding with isolated placental adherent cells.

The placental adherent cells (e.g., placental adherent cells) provided herein can also be seeded onto, or contacted with, a physiologically-acceptable ceramic material including, but not limited to, mono-, di-, tri-, alpha-tri-, beta-tri-, and tetra-calcium phosphate, hydroxyapatite, fluoroapatites, calcium sulfates, calcium fluorides, calcium oxides, calcium carbonates, magnesium calcium phosphates, biologically active glasses such as BIOGLASS®, and mixtures thereof. Porous biocompatible ceramic materials currently commercially available include SURGIBONE® (CanMedica Corp., Canada), ENDOBON® (Merck Biomaterial France, France), CEROS® (Mathys, AG, Bettlach, Switzerland), and mineralized collagen bone grafting products such as HEALOS™ (DePuy, Inc., Raynham, Mass.) and VITOSS®, RHAKOSS™, and CORTOSS® (Orthovita, Malvern, Pa.). The framework can be a mixture, blend or composite of natural and/or synthetic materials.

In one embodiment, the isolated placental adherent cells are seeded onto, or contacted with, a suitable scaffold at about 0.5×10⁶ to about 8×10⁶ cells/mL.

### 5.9 Immortalized Placental Cell Lines

Mammalian placental adherent cells, e.g., placental adherent cells, can be conditionally immortalized by transfection with any suitable vector containing a growth-promoting gene, that is, a gene encoding a protein that, under appropriate conditions, promotes growth of the transfected cell, such that the production and/or activity of the growth-promoting protein is regulatable by an external factor. In a preferred embodiment the growth-promoting gene is an oncogene such as, but not limited to, v-myc, N-myc, c-myc, p53, SV40 large T antigen, polyoma large T antigen, Ela adenovirus or E7 protein of human papillomavirus.

External regulation of the growth-promoting protein can be achieved by placing the growth-promoting gene under the control of an externally-regulatable promoter, e.g., a promoter the activity of which can be controlled by, for example, modifying the temperature of the transfected cells or the composition of the medium in contact with the cells. In one embodiment, a tetracycline (tet)-controlled gene expression system can be employed (see Gossen et al., Proc. Natl. Acad. Sci. USA 89:5547-5551, 1992; Hoshimaru et al., Proc. Natl. Acad. Sci. USA 93:1518-1523, 1996). In the absence of tet, a tet-controlled transactivator (tTA) within this vector strongly activates transcription from ph_{CMV*1}, a minimal promoter from human cytomegalovirus fused to tet operator sequences. tTA is a fusion protein of the repressor (tetR) of the transposon-10-derived tet resistance operon of Escherichia coli and the acidic domain of VP16 of herpes simplex virus. Low, non-toxic concentrations of tet (e.g., 0.01-1.0 μg/mL) almost completely abolish transactivation by tTA.

In one embodiment, the vector further contains a gene encoding a selectable marker, e.g., a protein that confers drug resistance. The bacterial neomycin resistance gene (neo^{R}) is one such marker that may be employed within the present methods. Cells carrying neo^{R} may be selected by means known to those of ordinary skill in the art, such as the addition of, e.g., 100-200 μg/mL G418 to the growth medium.

Transfection can be achieved by any of a variety of means known to those of ordinary skill in the art including, but not limited to, retroviral infection. In general, a cell culture may be transfected by incubation with a mixture of conditioned medium collected from the producer cell line for the vector and DMEM/F12 containing N2 supplements. For example, a placental cell culture prepared as described above may be infected after, e.g., five days in vitro by incubation for about 20 hours in one volume of conditioned medium and two volumes of DMEM/F12 containing N2 supplements. Transfected cells carrying a selectable marker may then be selected as described above.

Following transfection, cultures are passaged onto a surface that permits proliferation, e.g., allows at least 30% of the cells to double in a 24 hour period. Preferably, the substrate is a polyornithine/laminin substrate, consisting of tissue culture plastic coated with polyornithine (10 μg/mL) and/or laminin (10 μg/mL), a polylysine/laminin substrate or a surface treated with fibronectin. Cultures are then fed every 3-4 days with growth medium, which may or may not be supplemented with one or more proliferation-enhancing factors. Proliferation-enhancing factors may be added to the growth medium when cultures are less than 50% confluent.

The conditionally-immortalized placental cell lines can be passaged using standard techniques, such as by trypsinization, when 80-95% confluent. Up to approximately the twentieth passage, it is, in some embodiments, beneficial to maintain selection (by, for example, the addition of G418 for cells containing a neomycin resistance gene). Cells may also be frozen in liquid nitrogen for long-term storage.

Clonal cell lines can be isolated from a conditionally-immortalized human placental cell line prepared as described above. In general, such clonal cell lines may be isolated using standard techniques, such as by limit dilution or using cloning rings, and expanded. Clonal cell lines may generally be fed and passaged as described above.

Conditionally-immortalized human placental cell lines, which may, but need not, be clonal, may generally be induced to differentiate by suppressing the production and/or activity of the growth-promoting protein under culture conditions that facilitate differentiation. For example, if the gene encoding the growth-promoting protein is under the control of an externally-regulatable promoter, the conditions, e.g., temperature or composition of medium, may be modified to suppress transcription of the growth-promoting gene. For the tetracycline-controlled gene expression system discussed above, differentiation can be achieved by the addition of tetracycline to suppress transcription of the growth-promoting gene. In general, 1 μg/mL tetracycline for 4-5 days is sufficient to initiate differentiation. To promote further differentiation, additional agents may be included in the growth medium.

### 5.10 Kits

In another aspect, provided herein are kits, suitable for the treatment of an individual who has a disease or disorder of the circulatory system, comprising, in a container separate from remaining kit contents, tissue culture plastic adherent multipotent placental adherent cells, e.g., placental stem cells or placental multipotent cells, e.g., the cells described in Section 5.3, above (placental adherent cells), and instructions for use. Preferably, the placental adherent cells are provided in a pharmaceutically-acceptable solution, e.g., a solution suitable for intralesional administration or a solution suitable for intravenous administration. In certain embodiments, the placental stem cells or placental multipotent cells are any of the CD10⁺, CD34⁻, CD105⁺ placental adherent cells described herein, e.g., CD10⁺, CD34⁻, CD105⁺, CD200⁺ placental adherent cells or CD10⁺, CD34⁻, CD45⁻, CD90⁺, CD105⁺, CD200⁺ placental adherent cells.

In certain embodiments, the kits comprise one or more components that facilitate delivery of the placental adherent cells to the individual. For example, in certain embodiments, the kit comprises components that facilitate intralesional delivery of the placental adherent cells to the individual. In such embodiments, the kit can comprise, e.g., syringes and needles suitable for delivery of cells to the individual, and the like. In such embodiments, the placental adherent cells may be contained in the kit in a bag, or in one or more vials. In certain other embodiments, the kit comprises components that facilitate intravenous or intra-arterial delivery of the placental adherent cells to the individual. In such embodiments, the placental adherent cells may be contained, e.g., within a bottle or bag (for example, a blood bag or similar bag able to contain up to about 1.5 L solution comprising the cells), and the kit additionally comprises tubing and needles suitable for the delivery of cells to the individual.

Additionally, the kit may comprise one or more compounds that reduce pain or inflammation in the individual (e.g., an analgesic, steroidal or non-steroidal anti-inflammatory compound, or the like. The kit may also comprise an antibacterial or antiviral compound (e.g., one or more antibiotics), a compound to reduce anxiety in the individual (e.g., alaprazolam), a compound that reduces an immune response in the individual (e.g., cyclosporine A), an antihistamine (diphenhydramine, loratadine, desloratadine, quetiapine, fexofenadine, cetirizine, promethazine, chlorepheniramine, levocetirizine, cimetidine, famotidine, ranitidine, nizatidine, roxatidine, lafutidine, or the like).

Additionally, the kit can comprise disposables, e.g., sterile wipes, disposable paper goods, gloves, or the like, which facilitate preparation of the individual for delivery, or which reduce the likelihood of infection in the individual as a result of the administration of the placental adherent cells.

### 6. EXAMPLES

### 6.1 Example 1: Phenotypic Characterization of Placental Derived Adherent Cells

This example demonstrates secretion of angiogenic factors by placental adherent cells (CD10⁺, CD34⁻, CD105⁺, CD200⁺ placental stem cells, also called placental adherent cells).

Secretome Profiling for Evaluation of Angiogenic Potency of Placental Derived Adherent Cells

MulitplexBead Assay: Placental derived adherent cells at passage 6 were plated at equal cell numbers in growth medium and conditioned media were collected after 48 hours. Simultaneous qualitative analysis of multiple angiogenic cytokines/growth factors in cell-conditioned media was performed using magnetic bead-based multiplex assays (Bio-Plex Pro™, Bio-Rad, CA) assays are that allow the measurement of angiogenic biomarkers in diverse matrices including serum, plasma, and cell/tissue culture supernatants. The principle of these 96-well plate-formatted, bead-based assays is similar to a capture sandwich immunoassay. An antibody directed against the desired angiogenesis target is covalently coupled to internally dyed beads. The coupled beads are allowed to react with a sample containing the angiogenesis target. After a series of washes to remove unbound protein, a biotinylated detection antibody specific for a different epitope is added to the reaction. The result is the formation of a sandwich of antibodies around the angiogenesis target. Streptavidin-PE is then added to bind to the biotinylated detection antibodies on the bead surface. In brief, Multiplex assays were performed according to manufacturer's instructions and the amount of the respective angiogenic growth factors in the conditioned media was evaluated.

ELISAs: Quantitative analysis of single angiogenic cytokines/growth factors in cell-conditioned media was performed using commercially available kits from R&D Systems (Minneapolis, Minn.). In brief, ELISA assays were performed according to manufacturer's instructions and the amount of the respective angiogenic growth factors in the conditioned media was evaluated.

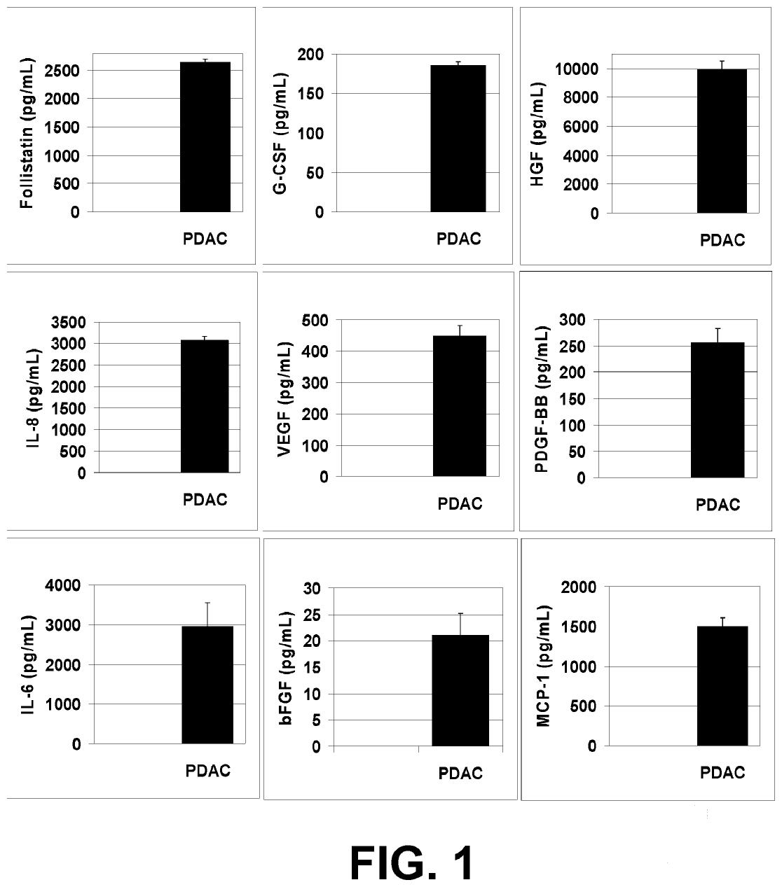

The level of secretion of various angiogenic proteins by placental adherent cell is shown in FIG. 1.

TABLE 1
Multiplex and ELISA results for angiogenic markers

| Placental Adherent | | | Secretome Analysis |
|---|---|---|---|
| Cell Marker | Positive | Negative | ELISA, Multiplex |
| ANG | X | | X |
| EGF | X | | X |
| ENA-78 | X | | X |
| FGF2 | X | | X |
| Follistatin | X | | X |
| G-CSF | X | | X |
| GRO | X | | X |
| HGF | X | | X |
| IL-6 | X | | X |
| IL-8 | X | | X |
| Leptin | X | | X |
| MCP-1 | X | | X |
| MCP-3 | X | | X |
| PDGFB | X | | X |
| PLGF | X | | X |
| Rantes | X | | X |
| TGFB1 | X | | X |
| Thrombopoietin | X | | X |
| TIMP1 | X | | X |
| TIMP2 | X | | X |
| uPAR | X | | X |
| VEGF | X | | X |
| VEGFD | X | | X |

In a separate experiment, placental adherent cells were confirmed to also secrete angiopoietin-1, angiopoietin-2, PECAM-1 (CD31; platelet endothelial cell adhesion molecule), laminin and fibronectin.

### 6.2 Example 2: Functional Characterization of Placental Cells

This Example demonstrates different characteristics of placental adherent cells (CD10⁺, CD34⁻, CD105⁺, CD200⁺ placental stem cells, also called placental adherent cells) associated with angiogenesis and differentiation capability.

### 6.2.1 HUVEC Tube Formation for Evaluation of Angiogenic Potency of Placental Adherent Cells

Human Umbilical Vein Endothelial Cells (HUVEC) were subcultured at passage 3 or less in EGM-2 medium (Cambrex, Fast Rutherford, N.J.) for 3 days, and harvested at a confluency of approximately 70%-80%. HUVEC were washed once with basal medium/antibiotics (DMEM/F12 (Gibco)) and resuspended in the same medium at the desired concentration. HUVEC were used within 1 hour of preparation. Human placental collagen (HPC) was brought to a concentration of 1.5 mg/mL in 10 mM HCl (pH 2.25), was neutralized with buffer to pH 7.2, and kept on ice until used. The HPC was combined with the HUVEC suspension at a final cell concentration of 4000 cells/μL. The resulting HUVEC/HPC suspension was immediately pipetted into 96-well plates at 3 μL per well (plate perimeter must be pre-filled with sterile PBS to avoid evaporation, n=5 per condition). HUVEC drops were incubated at 37° C. and 5% CO₂ for 75-90 minutes without medium addition to allow for collagen polymerization. Upon completion of “dry” incubation, each well was gently filled with 200 μL of conditioned placental adherent cell medium (n=2 cell lines) or control medium (e.g., DMEM/F12 as the negative control, and EGM-2 as the positive control) and incubated at 37° C. and 5% CO₂ for 20 hrs. Conditioned medium was prepared by incubating placental adherent cells at passage 6 in growth medium for 4-6 hours; after attachment and spreading, the medium was changed to DMEM/F12 for 24 hours. After incubation, the medium was removed from the wells without disturbing the HUVEC drops and the wells were washed once with PBS. The HUVEC drops were then fixed for 10 seconds and stained for 1 minute using a Diff-Quik cell staining kit and subsequently rinsed 3× times with sterile water. The stained drops were allowed to air dry and images of each well were acquired using the Zeiss SteReo Discovery V8 microscope. The images were then analyzed using the computer software package ImageJ and/or MatLab. Images were converted from color to 8-bit grayscale images and thresholded to convert to a black and white image. The image was then analyzed using the particle analysis features, which provided pixel density data, including count (number of individual particles), total area, average size (of individual particles), and area fraction, which equates to the amount endothelial tube formation in the assay.

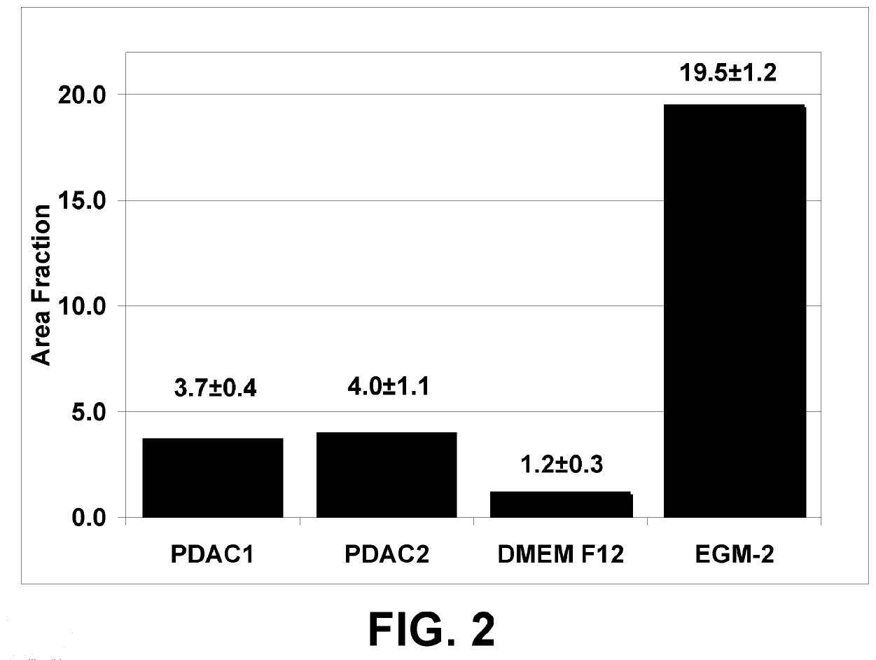

The conditioned medium exerted an angiogenic effect on endothelial cells, as demonstrated by the induction of tube formation (see FIG. 2).

### 6.2.2 HUVEC Migration Assay

This experiment demonstrated the angiogenic capacity of placental derived adherent cells. HUVECs were grown to monolayer confluence in a fibronectin (FN)-coated 12-well plate and the monolayer was “wounded” with a 1 mL plastic pipette tip to create an acellular line across the well. HUVEC migration was tested by incubating the “wounded” cells with serum-free conditioned medium (EBM2; Cambrex) obtained from placental adherent cells after 3 days of growth. EBM2 medium without cells was used as the control. After 15 hours, the cell migration into the acellular area was recorded (n=3) using an inverted microscope. The pictures were then analyzed using the computer software package ImageJ and/or MatLab. Images were converted from color to 8-bit grayscale images and thresholded to convert to a black and white image. The image was then analyzed using the particle analysis features, which provided pixel density data, including count (number of individual particles), total area, average size (of individual particles), and area fraction, which equates to the amount endothelial migration in the assay. The degree of cell migration was scored against the size of the initially recorded wound line and the results were normalized to 1×10⁶ cells.

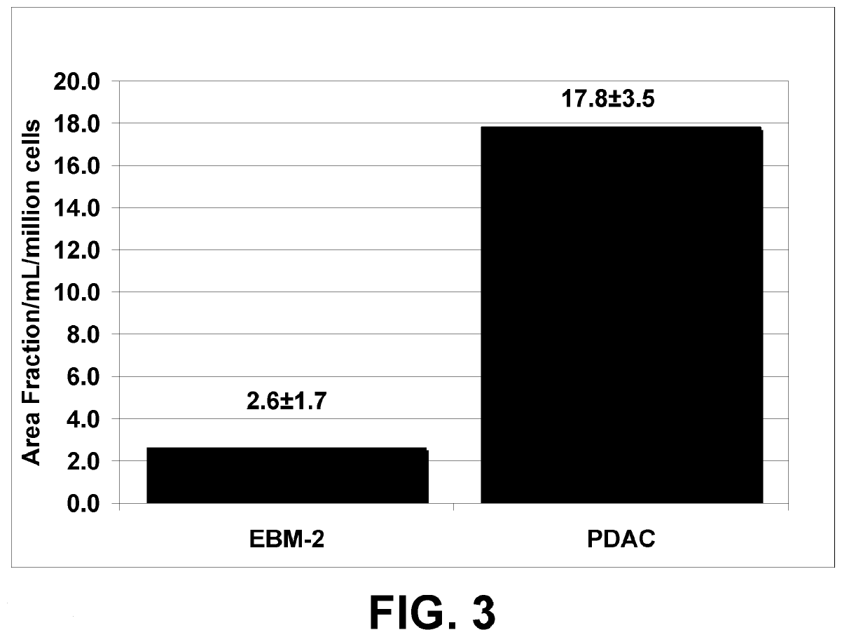

The trophic factors secreted by placental derived adherent cells exerted angiogenic effects on endothelial cells, as demonstrated by the induction of cell migration (FIG. 3).

In a separate experiment, HUVECs were cultured in the bottom of 24 well-plates for overnight establishment in EGM2, followed by a half-day starvation in EBM. Concurrently, media-cultured placental adherent cell were thawed and cultured in transwells (8 μM) overnight. After the EC starvation, the conditioned serum-free DMEM, along with the transwell, was transferred over to the ECs for overnight proliferation. 4 replicates were included in each experiment, and proliferation after 24 hrs was assessed with Promega's Cell Titer Glo Assay. EBM-2 medium was used as the negative control, and EGM-2 was used as the positive control. Error bars denote standard deviations of analytical replicates (n=3).

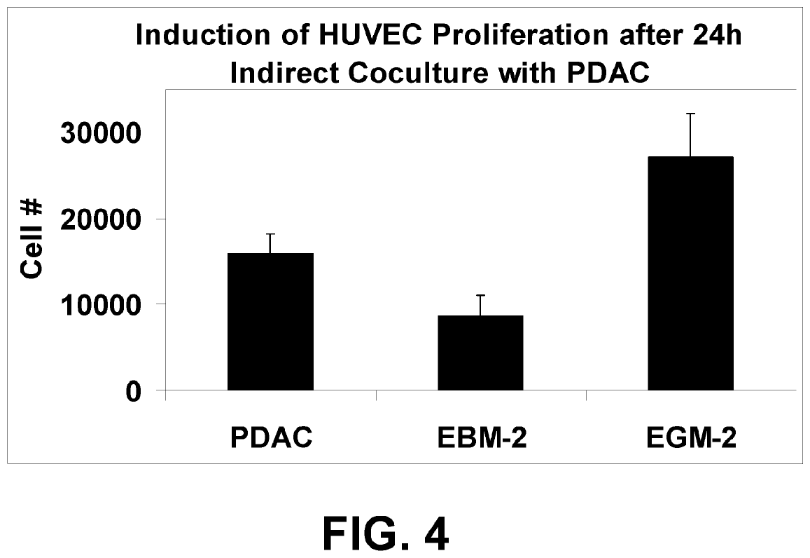

The trophic factors secreted by placental adherent cells resulted in an increase in HUVEC cell number, which is indicative of HUVEC proliferation. See FIG. 4.

### 6.2.3 Tube Formation for Evaluation of Angiogenic Potency of Placental Derived Adherent Cells

placental adherent cells were grown either in growth medium without VEGF or EGM2-MV with VEGF to evaluate the angiogenic potency of the cells in general, as well as the effect of VEGF on the differentiation potential of the cells. HUVECs, as control cells for tube formation, were grown in EGM2-MV. The cells were cultured in the respective media for 4 to 7 days until they reached 70-80% confluence. Cold (4° C.) MATRIGEL™ solution (50A; BD Biosciences) was dispensed into wells of a 12-well plate and the plate was incubated for 60 min at 37° C. to allow the solution to gel. The placental adherent cell and HUVEC cells were trypsinized, resuspended in the appropriate media (with and without VEGF) and 100 μL of diluted cells (1 to 3×10⁴ cells) were added to each of the MATRIGEL™-containing wells. The cells on the polymerized MATRIGEL™, in the presence or absence of 0.5 to 100 ng VEGF, were placed for 4 to 24 hours in a 5% CO₂ incubator at 37° C. After incubation the cells were evaluated for signs of tube formation using standard light microscopy.

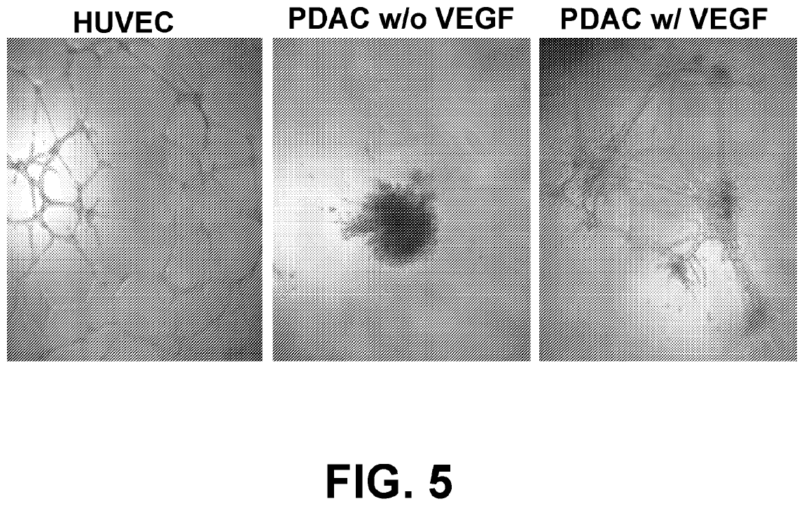

placental adherent cells displayed minimal tube formation in the absence of VEGF, but were induced/differentiated to form tube-like structures through stimulation with VEGF. See FIG. 5.

### 6.2.4 Hypoxia Responsiveness for Evaluation of Angiogenic Potency of Placental Derived Adherent Cells

To evaluate the angiogenic functionality of endothelial cells and/or endothelial progenitors, cells can be assessed in regard to their capability to secrete angiogenic growth factors under hypoxic and normoxic conditions. Culture under hypoxic conditions usually induces an increased secretion of angiogenic growth factors by either endothelial cells or endothelial progenitor cells, which can be measured in the conditioned media. Placental derived adherent cells were plated at equal cell numbers in their standard growth medium and grown to approximately 70-80% confluence. Subsequently, the cells were switched to serum-free medium (EBM-2) and incubated under normoxic (21% 02) or hypoxic (1% 02) conditions for 24 h. The conditioned media were collected and the secretion of angiogenic growth factors was analyzed using commercially available ELISA kits from R&D Systems. The ELISA assays were performed according to manufacturer's instructions and the amount of the respective angiogenic growth factors (VEGF and IL-8) in the conditioned media was normalized to 1×10⁶ cells.

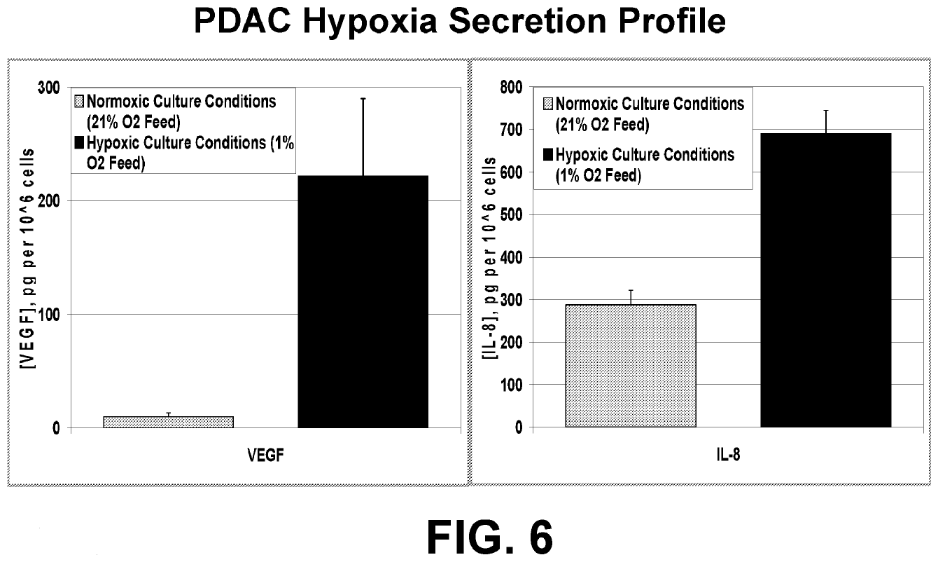

Placental derived adherent cells displayed elevated secretion of various angiogenic growth factors under hypoxic conditions. See FIG. 6.

### 6.2.5 HUVEC Response to Placental Adherent Cell-Conditioned Medium

Placental adherent cells were cultured for 48 hours in growth medium containing 60% DMEM-LG (Gibco); 40% MCBD-201 (Sigma); 2% FBS (Hyclone Labs), 1× insulin-transferrin-selenium (ITS); 10 ng/mL linoleic acid-bovine serum albumin (LA-BSA); 1 n-dexamethasone (Sigma); 100 μM ascorbic acid 2-phosphate (Sigma); 10 ng/mL epidermal growth factor (R & D Systems); and 10 ng/mL platelet-derived growth factor (PDGF-BB) (R & D Systems), and then cultured for an additional 48 hrs in serum-free media. Conditioned medium from placental adherent cell culture was collected and used to stimulate serum-starved HUVECs for 5, 15, and 30 minutes. The HUVECs were subsequently lysed and stained with a BD™ CBA (Cytometric Bead Assay) Cell Signaling Flex Kit (BD Biosciences) for phosphoproteins known to play a role in angiogenic pathway signaling. placental adherent cells were found to be strong activators of AKT-1 (which inhibits apoptotic processes), AKT-2 (which is an important signaling protein in the insulin signaling pathway, and ERK 1/2 cell proliferation pathways in HUVECs. These results further demonstrate the angiogenic capability of placental adherent cells.

### 6.3 Example 3: Induction of Angiogenesis by Placental Adherent Cells

This Example demonstrates that CD34⁻, CD10⁺, CD105⁺, CD200⁺ placental adherent cells, as described in Example 1 above, promote angiogenesis in an in vivo assay using chick chorioallantoic membrane (CAM).

Two separate CAM assays were conducted. In the first CAM assay, intact cell pellets from different preparations of placental adherent cells were evaluated. In the second CAM assay, supernatants of different placental adherent cell preparations were evaluated. Fibroblast growth factor (bFGF) was used as a positive control, and MDA-MB-231 human breast cancer cells as a reference, vehicle and medium controls were used as negative controls. The endpoint of the study was to determine the blood vessel densities of all treatment and control groups.

### 6.3.1 CAM Assay Using Placental Adherent Cells

Placental adherent cells, prepared as described in Example 1 above and cryopreserved, were used. Placental adherent cells were thawed for dosing and the number of cells dosed on the CAM was determined.

Study Design: The study included 5 groups with 10 embryos in each group. The design of the study is described in Table 2.

TABLE 2
Study groups, chick chorioallantoic membrane angiogenesis assay

| Group No. | # of embryos | Treatment | End Point |
|---|---|---|---|
| 1 | 10 | Vehicle control | Blood vessel density |
| | | (40 μl of PBS/ | score |
| | | MATRIGEL ™ | |
| | | mixture, 1:1 by | |
| | | volume) | |
| 2 | 10 | Positive control, | Same as group 1 |
| | | treated with bFGF | |
| | | (100 ng/CAM in | |
| | | 40 μl of DMEM/ | |
| | | MATRIGEL ™ | |
| | | mixture, 1:1) | |
| 3 | 10 | Medium control | Same as group 1 |
| | | (40 μl of DMEM) | |
| 4 | 10 | Placental adherent | Same as group 1 |
| | | cells (40 μl) | |
| 5 | 10 | MDA-MB-231 cells | Same as group 1 |
| | | P34, Lot No. 092608 | |

CAM Assay Procedure: Fresh fertile eggs were incubated for 3 days in a standard egg incubator at 37° C. for 3 days. On Day 3, eggs were cracked under sterile conditions and embryos were placed into twenty 100 mm plastic plates and cultivated at 37° C. in an embryo incubator with a water reservoir on the bottom shelf Air was continuously bubbled into the water reservoir using a small pump so that the humidity in the incubator was kept constant. On Day 6, a sterile silicon “O” ring was placed on each CAM, and then placental adherent cells at a density of 7.69×10⁵ cells/40 μL of medium/MATRIGEL™ mixture (1:1) were delivered into each “O” ring in a sterile hood. Table 2 describes the number of cells used and the amount of medium added to each cell preparation for dosing. Vehicle control embryos received 40 μL of vehicle (PBS/MATRIGEL™, 1:1), positive controls received 100 ng/ml bFGF in 40 μL of DMEM medium/MATRIGEL™ mixture (1:1), and medium controls received 40 μL of DMEM medium alone. Embryos were returned to the incubator after each dosing was completed. On Day 8, embryos were removed from the incubator and kept at room temperature while blood vessel density was determined under each “0” ring using an image capturing system at a magnification of 100×

Blood vessel density was measured by an angiogenesis scoring system that used arithmetic numbers 0 to 5, or exponential numbers 1 to 32, to indicate the number of blood vessels present at the treatment sites on the CAM. Higher scoring numbers represented higher vessel density, while 0 represented no angiogenesis. The percent of inhibition at each dosing site was calculated using the score recorded for that site divided by the mean score obtained from control samples for each individual experiment. The percent of inhibition for each dose of a given compound was calculated by pooling all results obtained for that dose from 8-10 embryos.

TABLE 3
Amount of medium added to each cell preparation for
normalization of the final cell suspension for dosing

| | | Normalization with | Final Volume |
|---|---|---|---|
| | | DMEM and | of Cell |
| Cell Line | Pellet size | MATRIGEL ™ | Suspension |
| Placental adherent | 260 uL | 0 μL + 260 μL | 520 μL |
| cell | | MATRIGEL ™ | |
| MDA-MB-231 | 40 uL | 220 μL + 260 μL | 520 μL |
| | | MATRIGEL ™ | |

Placental adherent cells were used at Passage 6.

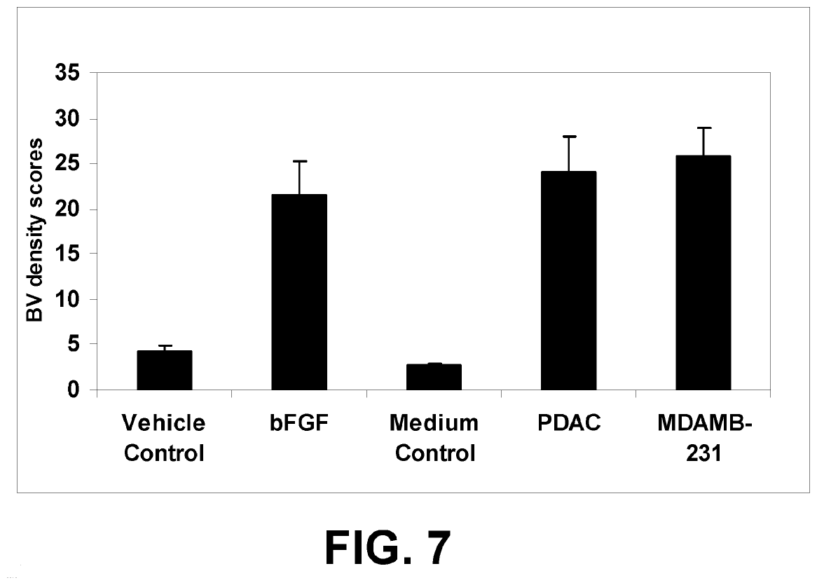

Results: The results of blood vessel density scores are presented in FIG. 7. The results clearly indicate that the blood vessel density scores of chick chorioallantoic membranes treated with placental adherent cell suspensions, or 100 ng/mL of bFGF, or MDAMB231 breast cancer cell suspensions were statistically significantly higher compared to those of the vehicle control CAMs (P<0.001, Student's “t” test). The medium used for culturing placental adherent cells (negative control) did not have any effect on the blood vessel density. Further, the induction of blood vessel density of placental adherent cell preparations showed some variation, but the variations were not statistically significant.

### 6.3.2 CAM Assay Using Placental Adherent Cell Supernatants

Supernatant samples from MDA-MB-231 cells and placental adherent cell were used in a second CAM assay as described above. bFGF and MDA-MB-231 supernatants were used as positive controls, medium and vehicle were used as negative controls.

Study Design: The study included five groups with 10 embryos in each group. The design of the study is described in Table 4.

TABLE 4
Study Design-CAM assay using cell supernatants

| Group | # of | | |
|---|---|---|---|
| No. | Embryos | Treatment | End Point |
| 1 | 10 | Vehicle control (40 μl | Blood vessel |
| | | of PBS/ MATRIGEL ™ | density score |
| | | mixture, 1:1 by volume) | |
| 2 | 10 | Positive control, treated | Same as group 1 |
| | | with bFGF (100 ng/ | |
| | | CAM in 40 μl of | |
| | | DMEM/MATRIGEL ™ | |
| | | mixture, 1:1) | |
| 3 | 10 | Medium control (40 μl | Same as group 1 |
| | | of DMEM) | |
| 4 | 10 | Supernatant of placental | Same as group 1 |
| | | adherent cells | |
| 5 | 10 | Supernatant of | Same as group 1 |
| | | MDAMB231 cells | |
| | | (P34) | |

Placental adherent cell supernatants were obtained from cells at Passage 6.

CAM Assay Procedure: The assay procedure was the same as described in section 5.3.1, above. The only difference was that supernatant from each stem cell preparation or from MDA-MB-231 cells was used as test material. For dosing, each supernatant was mixed with MATRIGEL™ (1:1 by volume) and 40 μL of the mixture was dosed to each embryo.

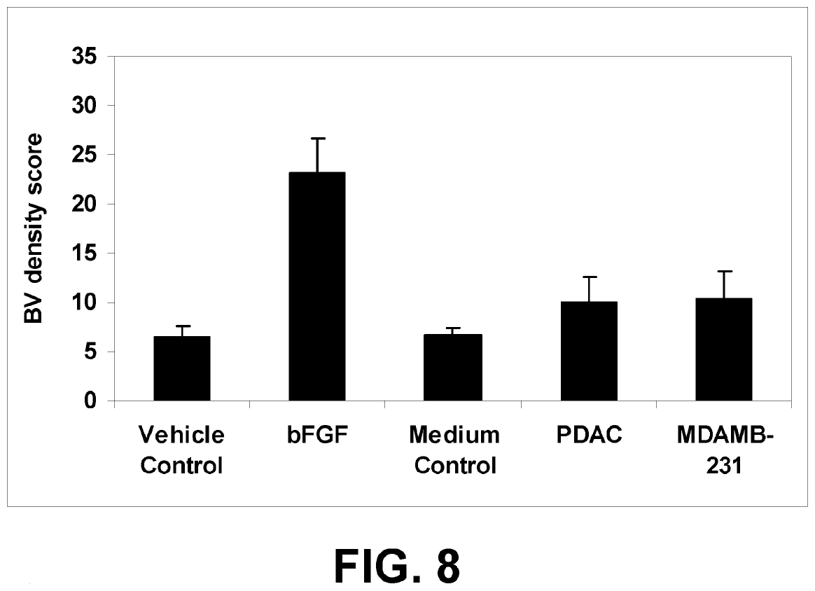

Results: Blood vessel density scores (see FIG. 8) indicate that the induction of blood vessel formation by the supernatant of each stem cell preparation differed. Supernatant samples from placental adherent cell showed significant effect on blood vessel induction with P<0.01, P<0.001, and P<0.02 (Student's “t” test) respectively. As expected, positive control bFGF also showed potent induction of blood vessel formation as seen above in the first CAM assay (P<0.001, Student's “t” test). However, supernatant from MDA-MB-231 human breast cancer cells did not show significant induction on blood vessel formation compared to the vehicle controls. As previously shown, culture medium alone did not have any effect.

### 6.4 Example 4: Systemic Effect of Placental Adherent Cells on Endothelial and Muscle Cells

### 6.4.1 Materials and Methods

Study design: A sample size of at least nine mice per group was used for laser Doppler blood flow analysis and for behavioral testing, four to five mice per group were used for X-ray angiography, and four to five mice per group were used for histological evaluation. Animals were randomly assigned to the experimental groups, and the experimentalists were blinded to the identity of the experimental groups while testing the multiple endpoints in this study. No outliers were excluded and all collected data were used in statistical analyses.

Mice: db/db mice (10-11 weeks old) and Balb/c, wild-type and athymic nude mice (10 to 12 weeks old), were purchased from Charles Rivers (Boston, MA, USA). All animal experiments conformed to National Institute of Health (NIH) and Association for Assessment and Accreditation of Laboratory Animal Care (AAALAC) guidelines.

Preparation of placental adherent cells and human dermal fibroblasts (HDF): CD34⁻, CD10⁺, CD105⁺, CD200⁺ placental adherent cells were prepared by mechanical and enzymatic digestion of human placental tissue obtained from a normal, full-term birth as described in Example 1 and in Liu et al. 2014, Clin. Transl. Immunology 3(e14). Placental adherent cells were expanded until passage 6 in proprietary medium and subsequently used for in vivo studies as previously described (Francki et al. 2015. J. Vasc. Surg. 15:901-915). HDF cells were obtained from Lonza (Walkersville, MD, USA) and were culture-expanded according to the manufacturer's instructions.

Mouse model of hind-limb ischemia (HLI): In the unilateral HLI model in db/db mice, the right femoral artery was ligated twice (proximal to the deep femoral artery) and transected between the ligatures. Vehicle, HDF (3×10⁴ cells), or placental adherent cells (3×10⁴ cells) were administered intramuscularly 24 hours post-ligation at two sites: the proximal and the distal side of the surgical wound (25 μl at each site, for a total of 50 μl per mouse). In the bilateral HLI model in db/db mice, the right femoral artery was ligated twice (distal to the deep femoral artery) and transected between the ligatures. Animals were treated with vehicle, VEGF (3.3 μg/animal; Sigma-Aldrich, St. Louis, MO, USA) or placental adherent cells (5×10⁴ cells) 48 hours post-ligation. The second injury, ligation of femoral artery (proximal to the deep femoral artery), was performed in the left hind-limb 23 days after the ligation and transection of femoral artery of the right hind-limb.

In T cell reconstitution experiments, CD4⁺ T cells were isolated from the spleen of wild-type Balb/c mice, and 5×10⁶ of CD4⁺ T cells were transferred intravenously to athymic nude Balb/c recipient mice. Two weeks after transplantation of CD4⁺ T cells, recipient mice were subjected to unilateral HLI followed by vehicle or placental adherent cells (5×10⁴ cells) treatment 24 hours later.

Blood flow measurement: Repeated hind-limb blood flow measurements over the region of interest (calf area) were obtained at baseline, immediately after surgery, and serially over 6 weeks using a non-contact Laser Doppler. In a unilateral HLI model, blood flow is expressed as a ratio of blood flow in the right (ischemic) to the left (normal) limb at each time point. In a bilateral ligation model, blood flow is expressed as the ratio of blood flow in the hindlimb at specific time points relative to the blood flow at baseline prior to the HLI injury.

Angiography: Mice were anesthetized by ketamine/Xylazine. Barium sulfate solution (2.5 ml) was infused into the infrarenal aorta after ligation of the proximal aorta and inferior vena cava, and the number of contrast-filled blood vessels was determined using X-Ray roentgen assay. Quantification of intersections between contrast-filled vessels and gridlines of the angiographs is presented as an angioscore, defined as the number of blocks on a grid filled by radiopaque dye.

Histology and immunohistochemistry: Hind-limb muscles were removed and fixed in HOPE (Hepes-glutamic acid buffer mediated Organic solvent Protection Effect) fixative (Polysciences, Inc, Warrington, PA) and embedded in paraffin. The paraffin blocks were cooled to 20° C. for 30 min and sectioned using a microtome at 5 μm thickness. Muscle anatomy was visualized by staining two serial sections with hematoxylin and eosin and evaluated microscopically for morphological changes. For immunostaining, sections of thigh muscles were first blocked with 10% horse donkey serum 2× casein, in PBS for 30 min at room temperature, then incubated at 4° C. overnight with primary antibodies and with secondary antibodies for 30 min at room temperature. For primary antibodies, rat anti-CD31 (1:100, Abcam, Cambridge, MA, USA), rabbit anti-αSMA (1:100, Abcam), rat anti-CD68 (1:100, ABD Serotec, Raleigh, NC, USA), and goat anti-arginase 1 (1:100, Santa Cruz, Dallas, TX, USA) were used. Appropriate secondary antibodies included goat-anti-rat IgG conjugated with Alexa Fluor 488 (1:500, Invitrogen, Carlsbad, CA, USA), goat-anti-rabbit IgG Alexa Fluor 594 (1:500, Invitrogen), chicken anti-rat IgY conjugated with Alexa Fluor 488 (1:500, invitrogen), and donkey anti-goat IgG conjugated Alexa Fluor 594 (1:500, Invitrogen). For nuclear staining, 600 nM DAPI solution (Sigma, St. Louis, MO, USA) was applied. Immunohistochemistry images were captured with a Nikon Eclipse microscope model E800 equipped with a high-resolution digital camera Nikon DXM1200F connected to PC equipped with NIS Elements software for image capture and archiving. For vessel density and morphometric analysis, three sections from each of four to five representative animals per treatment group were evaluated. Within each section, three non-overlapping fields were randomly selected and digitally captured at 20× magnification. The average cross-sectional area measured was 0.35 mm² per field.

Human macrophage in vitro differentiation: Human monocytes were isolated from blood using CD14⁺ magnetic beads (Miltenyi, San Diego, CA, USA). Monocytes were plated in 100 cm dishes in RPMI 1640 media supplemented with 100 ng/ml M-CSF and cultured for 7-8 days to derive MO macrophages. Cells were subsequently harvested with ACCUTASE® and plated in 6 well plates at 5×10⁵ cells per well. As assay controls, select wells were treated with 100 ng/ml LPS or 20 ng/ml IL-4 for 48 hours to polarize macrophages to M1 and M2, respectively. MO macrophages were continued in culture with RPMI 1640 media. Placental adherent cells were co-cultured with MO macrophages in transwells at a 1:1 ratio. After 48-hour treatment, macrophages were harvested with ACCUTASE® and analyzed by flow cytometry for mean fluorescent intensity (MFI) of CD206, CD80 and HLA-I.

Flow cytometry: All antibodies for flow cytometry staining were purchased from BD Biosciences (Biosciences, San Jose, CA, USA). Macrophages were washed with FACS wash buffer (PBS with 2% FBS) and labeled with antibodies by incubation at 4° C. for 30 min. Flow cytomety analysis was performed on a FACS Canto II (BD Biosciences). Data were acquired with FACSDiva software (BD Biosciences) and analyzed using FlowJo flow cytometry software (Tree Star, Ashland, OR, USA).

Statistical analysis: Data are expressed as mean±SEM. Statistical significance was determined by 2-tailed Student “t” test in all ex vivo studies. P values <0.05 were considered statistically significant. Two-way analysis of variance (ANOVA) with repeated measures was used to analyze differences between groups of mice in vivo. When significant treatment-related effects were observed (ANOVA; P<0.05), pair wise least significant difference (LSD) post-hoc analyses were performed.

Draining lymph node cell preparation and analysis: In unilateral HLI experiments performed in db/db mice, inguinal dLNs were collected 3, 7 or 14 days post ligation and transection of the femoral artery. Single cell suspension from dLNs were obtained by mechanical dissociation using OctoMACS dissociator (Miltenyi, San Diego, CA, USA) based on manufacturer's instructions. To measure gene expression and cytokine production, dLN cells were stimulated incomplete RPMI with PMA/Ionomycin (eBiosciences, San Diego, CA, USA) for 2 and 24 hours, respectively. Supernatants were analyzed using the Millipore Mouse MAP Cytokine/Chemokine Magnetic Bead Panel (MCYTMAG-70K-PX32).

Quantitative PCR analysis: For gene expression, individual Tagman gene expression assay for mouse were used for Gata3, Tbet (Applied Biosystems). Relative gene expression was normalized using GAPDH expression, and the fold change was calculated using the 2-ΔΔCt method.

Draining lymph node cell transplantation in db/db mice HLI model: In dLN cell transplantation experiments, HLI was created by unilateral ligation and transection of femoral artery in the right hind-limb of db/db mice and animals were treated with vehicle, HDF (5×10⁴ cells) or placental adherent cells (5×10⁴ cells) 24 hours after HLI. Fourteen days after treatment, the animals were sacrificed and inguinal dLNs were isolated from these donor animals. The dLN cells (1×10⁶ cells/animal) were administered intramuscularly into the recipient mice subjected to unilateral HLI 24 hours earlier. Blood flow was measured by laser Doppler, and the density of CD31+ or αSMA+ vessel was determined by immunohistochemistry.

### 6.4.2 Results

CD34⁻, CD10⁻, CD105⁺, CD200⁺ Placental Adherent Cells Improve Blood Perfusion and Collateral Vessel Formation.

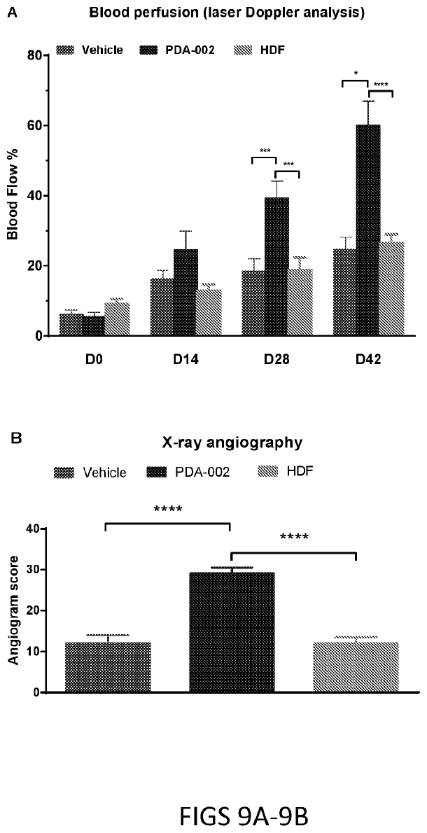

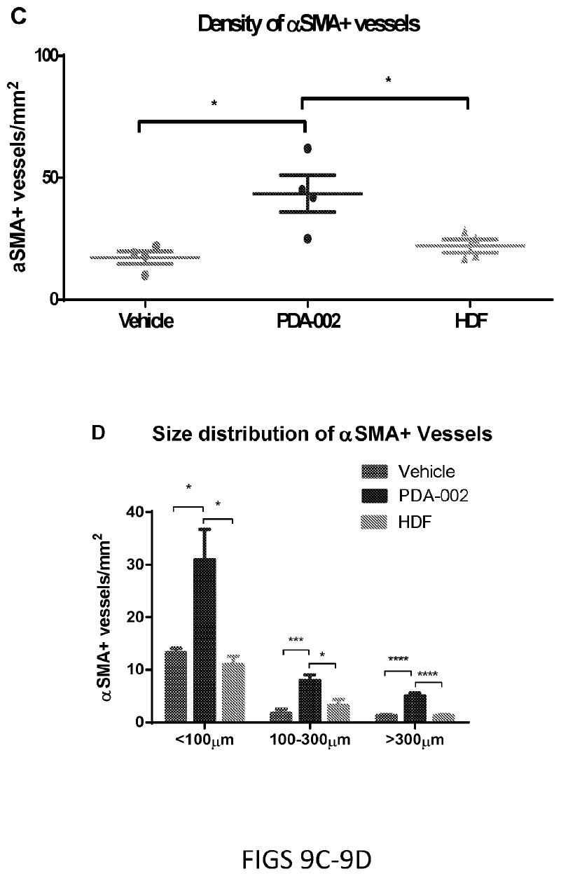

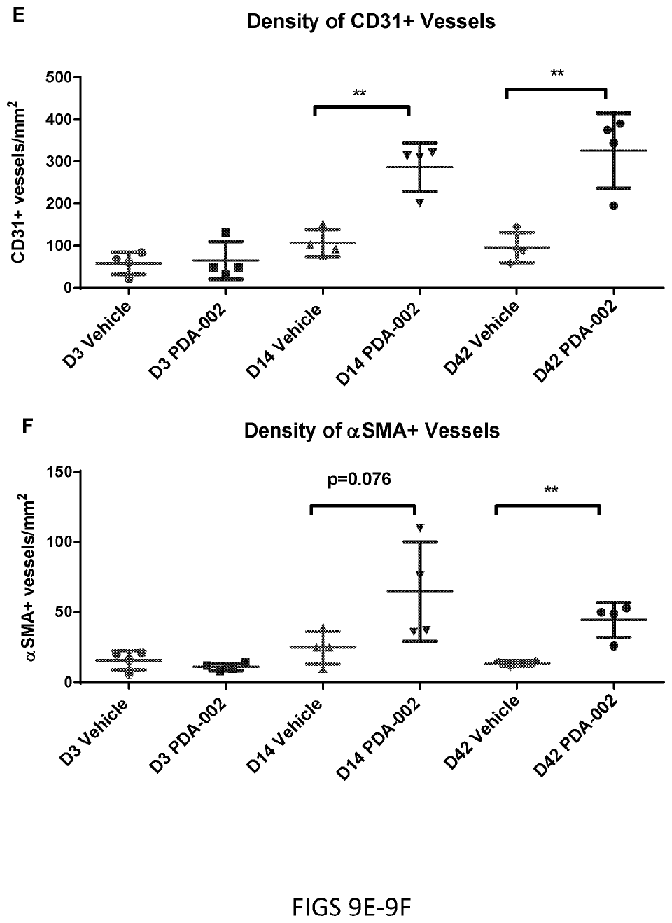

In vehicle-treated mice, blood flow in the injured limb dropped precipitously below 10% after HLI, and recovered to approximately 20% and 35% by days 14 and 42, respectively. Placental adherent cell treatment significantly improved blood flow compared to vehicle by day 28 (FIG. 9A). Human dermal fibroblast cells (HDF), used as a human cell line control, did not improve blood flow at any time point examined compared to vehicle, suggesting that placental adherent cell-induced arteriogenesis was not due to repair caused by a non-specific xenogeneic reaction. X-ray angiography also showed that animals treated with placental adherent cells had higher numbers of blood vessels than vehicle or HDF-treated animals (FIG. 9B).

Histological analysis of the upper limb muscles (quadriceps and adductors) at day 42 revealed that the placental adherent cells significantly increased the number of alpha smooth muscle actin (αSMA)⁺ blood vessels compared to vehicle and HDF (FIG. 9C). Size distribution analysis of αSMA⁺ vessels showed that the placental adherent cells increased not only the number of small vessels (<100 μm), but also large vessels (100-300 μm and >300 μm) (FIG. 9D). To better understand the time-course of the placental adherent cell treatment and collateral blood vessel development, the upper limb muscles from days 3, 14 and 42 were histologically examined for αSMA and for the endothelial marker CD31. On day 3, there was no difference of CD31⁺ or αSMA⁺ vessel density between placental adherent cell-treated and vehicle-treated animals. However, the placental adherent cells increased CD31⁺ or αSMA⁺ vessel density approximately three-fold higher than that in vehicle-treated animals by days 14 and 42 (FIG. 9E-F). Furthermore, the placental adherent cells increased the number of myofibers with centrally-located nuclei—a hallmark of regenerating myofiber—in the injured limb, suggesting that placental adherent cell treatment not only resulted in improvement in blood perfusion, but also enhanced muscle repair.

CD34⁻, CD10⁺, CD105⁺, CD200⁺ Placental Adherent Cells Modulate Macrophage Differentiation Toward an M2 Phenotype

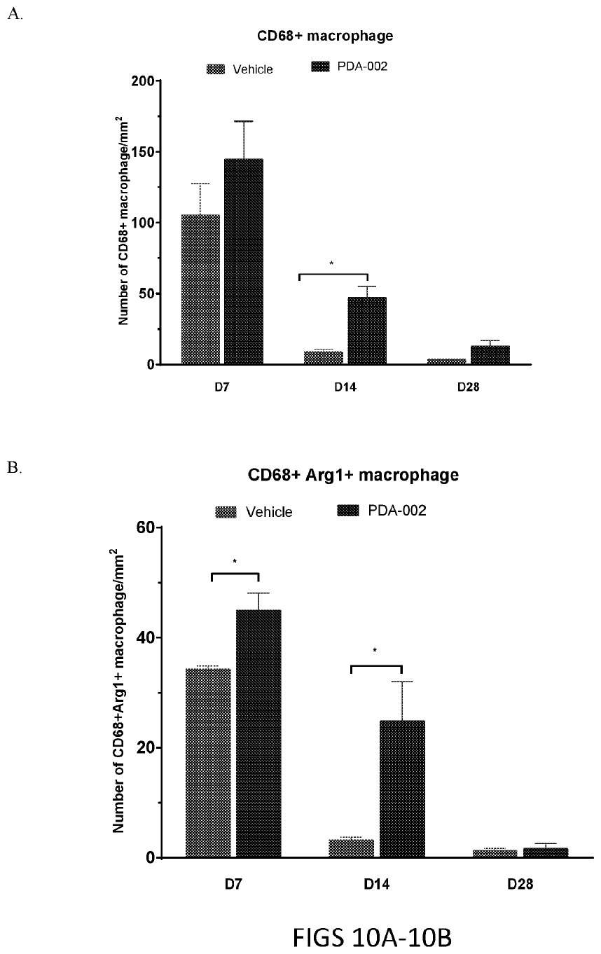

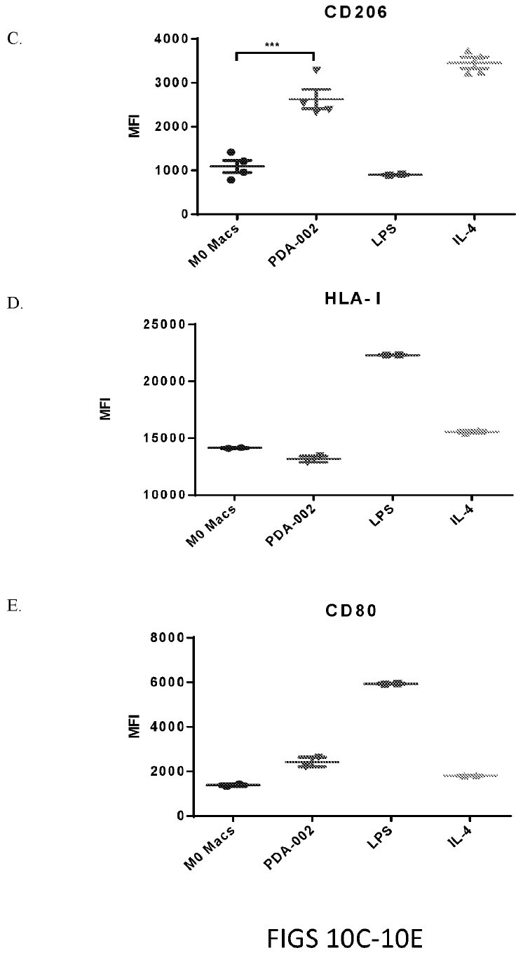

To understand whether placental adherent cell-enhanced arteriogenesis could be associated with the modulation of components of the immune response during ischemic injury, the phenotype and the tissue density of total infiltrating macrophages and M2-like macrophages in the upper limb ischemic tissue of db/db mice by their expression of CD68 and Arginase 1 (Arg1), respectively, was analyzed. No significant difference in the number of CD68⁺ total macrophages was detected in placental adherent cell-treated or vehicle-treated animals on Day 7. The number of CD68⁺ macrophages decreased dramatically from 104±20.4/mm² on Day 7 to 8±2.3/mm² on Day 14 in vehicle-treated animals, whereas a significantly higher number (46±8.3/mm²) of CD68⁺ macrophages was sustained by Day 14 in placental adherent cell-treated animals (FIG. 10A). Moreover, placental adherent cell treatment significantly increased the density of CD68⁺ Arg1⁺ M2-like macrophages by both Day 7 and Day 14 compared to the vehicle control group (FIG. 10B).

The ability of placental adherent cells to promote human macrophage differentiation toward an M2 phonotype in vitro was analyzed. MO macrophages derived from peripheral blood mononuclear cells (PBMC) were cultured with placental adherent cells in transwell plates for 48 hours. After coculture with placental adherent cells, MO macrophages showed a significant increase in CD206 expression similar to that detected in IL-4-induced M2 macrophages (FIG. 10C). Moreover, the expression of HLA-I (FIG. 10D) and CD80 (FIG. 10E) was also similar to IL-4-induced M2 macrophages, and significantly lower levels than those detected in LPS-induced M1 macrophages (FIG. 10D-E).

T Cells Play an Important Role in CD34⁻, CD10⁺, CD105⁺, CD200⁺ Placental Adherent Cell-Mediated Arteriogenesis and Modulation of Macrophages Toward M2 Phenotype

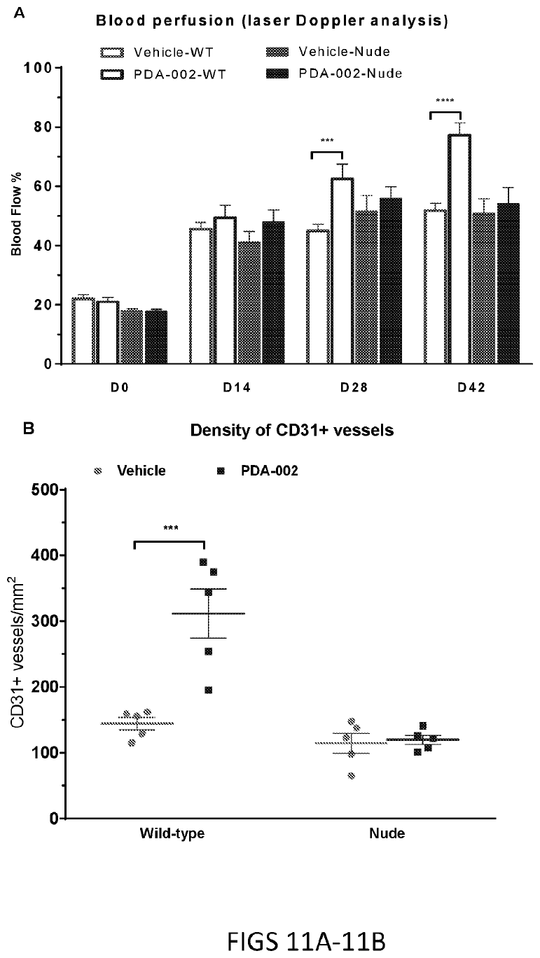

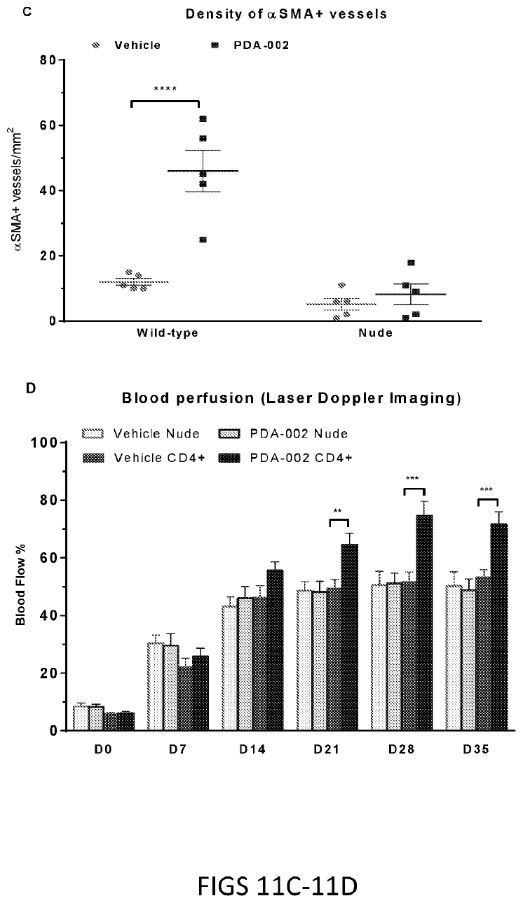

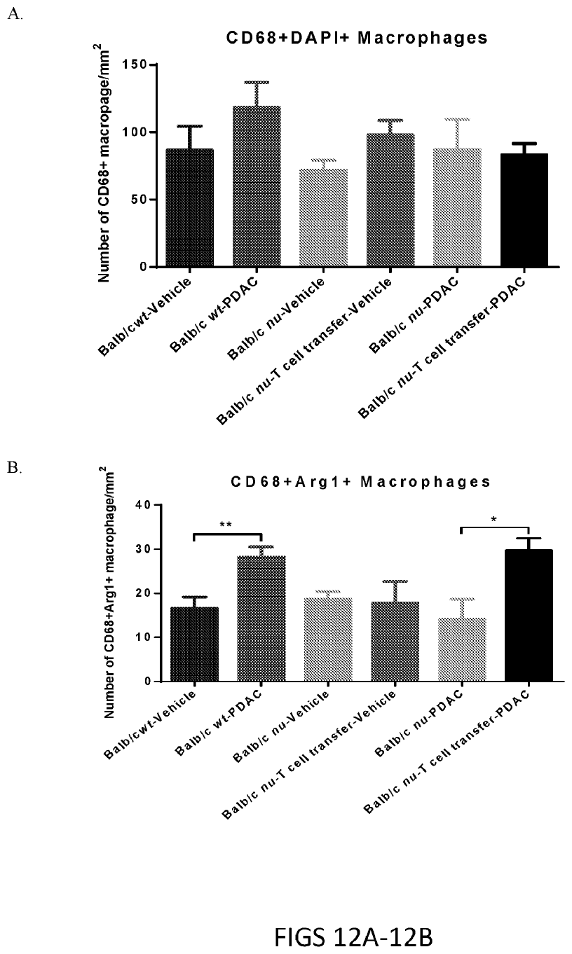

To assess the role of T cells in the results observed above, the arteriogenic effects of placental adherent cells in Balb/c wild-type and nude mice subjected to unilateral HLI was examined. Placental adherent cells significantly increased blood flow in wild-type mice by day 28; however, blood flow was not improved by the placental adherent cells in nude mice (FIG. 11A). Histological analysis confirmed that placental adherent cells were unable to increase CD31⁺ (FIG. 11B) or αSMA⁺ (FIG. 11C) blood vessel density compared to vehicle in nude mice. To further confirm the requirement of T cells in placental adherent cell-mediated arteriogenesis, placental adherent cell efficacy in a T cell reconstituted nude mouse HLI model was evaluated. Laser Doppler analysis showed that placental adherent cells significantly enhanced blood flow from day 21 through day 35 in these animals (FIG. 1 ID) indicating that T cell reconstitution restored placental adherent cells' efficacy in nude mice. Moreover, placental adherent cell treatment significantly increased the number of CD68⁺ Arg1⁺ M2-like macrophages 7 days after HLI compared to vehicle in wild-type and T cell reconstituted nude mice, but had no effect in nude mice. (FIGS. 12A and 12B).

Additional experiments were performed to identify the correlative biomarkers of the placental adherent cells' effect on functional T cell response, by analyzing the changes in functional T cell phenotype in the lymph nodes draining the ischemic tissue, following placental adherent cell treatment. Briefly, the data demonstrated that placental adherent cell treatment is associated with the induction of cytokine and gene expression signature of a Th2 response, and the resulting draining lymph node cells recapitulated pro-arteriogenic effects of placental adherent cells following intramuscular administration in the HLI model.

Overall, these data indicate that T cells are important for placental adherent cell-mediated arteriogenesis and placental adherent cell-induced M2 macrophage differentiation in ischemic tissue, correlated with induction of a pro-arteriogenic lymphocyte phenotype and biomarkers of Th2 type response.

CD34⁻, CD10⁺, CD105⁺, CD200⁺ Placental Adherent Cell Treatment Exerts Systemic Arteriogenesis

These data reveal that immunomodulation of T cells and macrophages is a key mechanism underlying placental adherent cell-mediated arteriogenesis. To evaluate whether placental adherent cells can exert systemic arteriogenic effects through M2 macrophage skewing in response to ischemic injury, a bilateral HLI model where a second HLI was created in the contralateral hind-limb 23 days after the first HLI injury and 21 days after cell injection into the first injured hind-limb was developed. The timing of the second HLI allowed for adequate, but not complete recovery of the first injured limb and ensured that the placental adherent cells were no longer present in the animal, based on the previously-characterized duration of the cells' in vivo persistence (Francki et al. 2015).

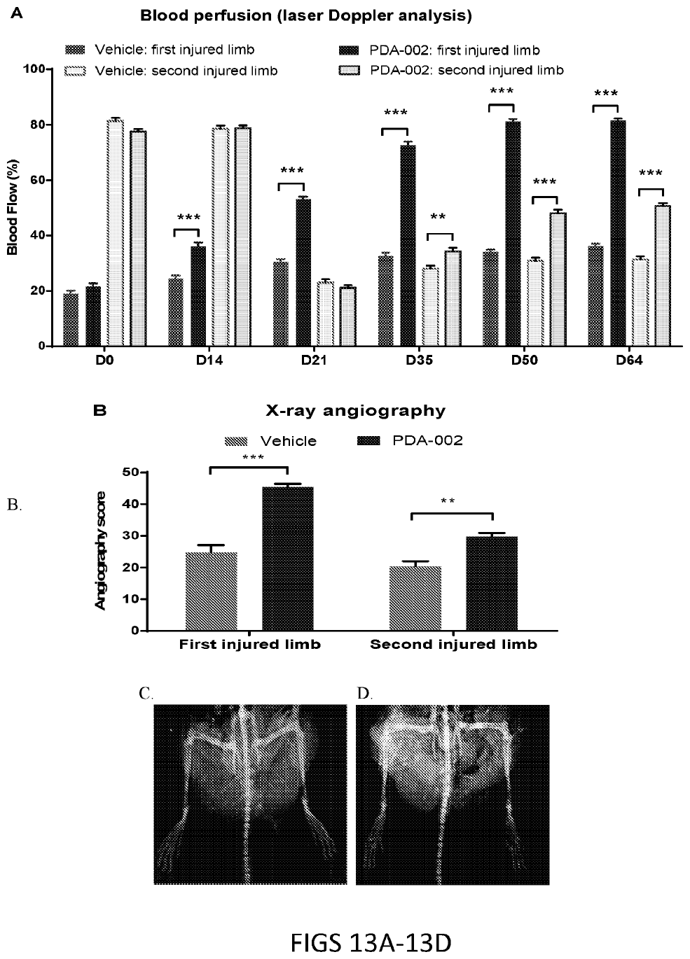

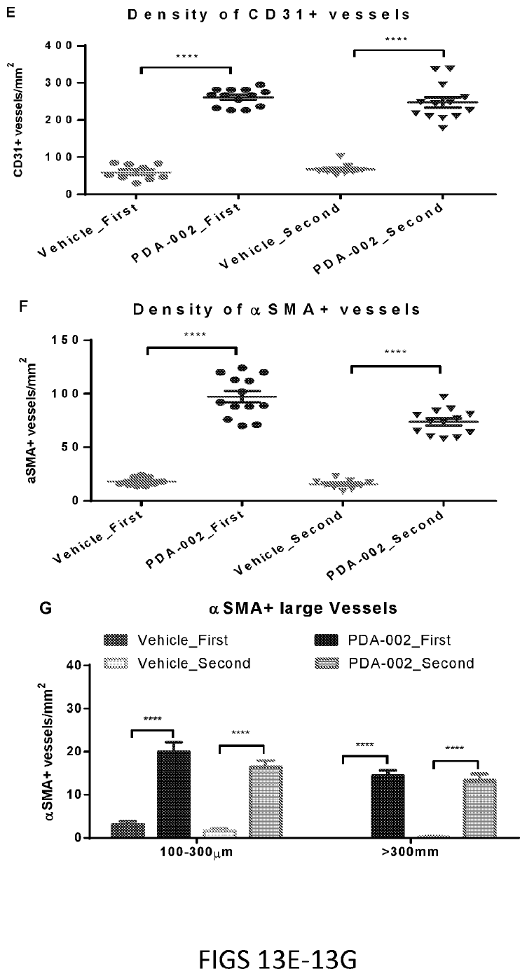

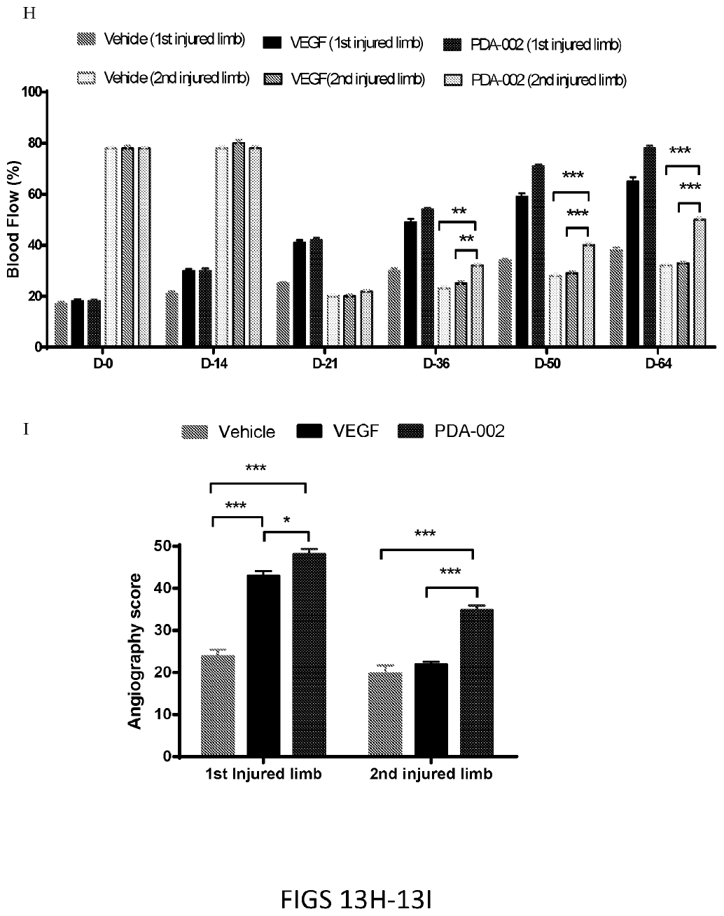

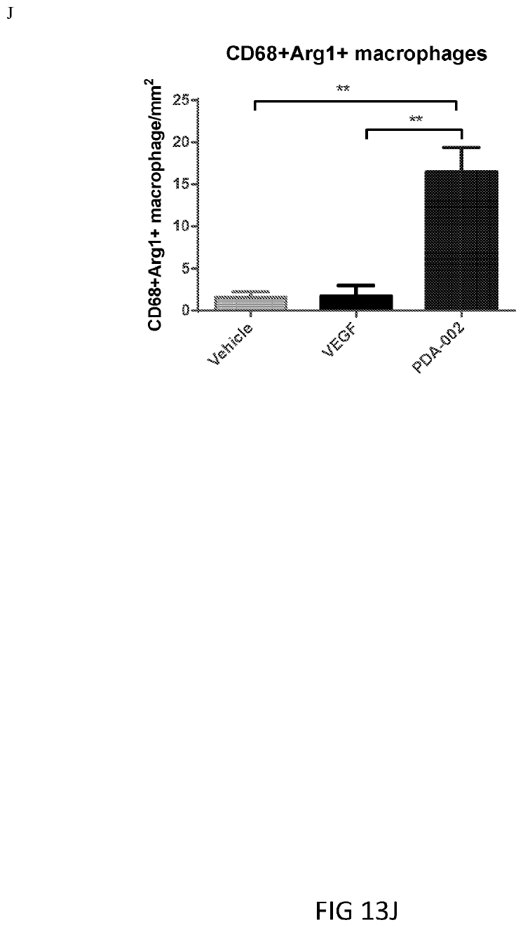

In the first injured limb, the placental adherent cells, injected 48 hours following surgery, significantly increased blood flow from day 14 through day 64 compared to vehicle. Surprisingly, these animals also demonstrated significantly improved blood flow in the second contralateral injury from day 35 (14 days after second HLI) through day 64, although the degree of blood flow restoration was weaker than that in the first injured limb (FIG. 13A). Angiography confirmed that placental adherent cells increased the number of blood vessels in both injured limbs (FIG. 13B-D). Histological analysis showed that the placental adherent cells increased CD31⁺, αSMA⁺ blood vessel density and αSMA⁺ large vessels (FIGS. 13E-G, respectively) in the ischemic tissue of both injured limbs. Arteriogenic efficacy of the placental adherent cells versus VEGF in the bilateral HLI model also was compared. Administration of the cells significantly increased blood flow and angioscore in both injured limbs, whereas the effect of VEGF was limited to the first injured limb where it was injected (FIG. 13H-I). CD68⁺ Arg1⁺ M2-like macrophages were significantly higher in the second injured limb 7 days after HLI in the placental adherent cell-treated animals compared to vehicle- or VEGF-treated animals (FIG. 13J), indicating that arteriogenesis in the second injured limb shares the same mechanisms as the first injured limb.

### 6.5 Example 5: Placental Adherent Cells Induce Lymphangiogenesis

LYVE-1 (lymphatic vessel endothelial hyaluronan receptor-1) is a protein characteristic of lymphatic vessels, which can be used to distinguish lymphatic vessels from blood vessels. db/db mice were subjected to HLI as described above, and 24 h later were injected in the ipsilateral hindlimb with approximately 50,000 CD34⁻, CD10⁺, CD105⁺, CD200⁺ placental adherent cells or vehicle. Inguinal draining lymph nodes were collected at days 3 and 14 for immunohistochemical examination. Mice receiving placental adherent cells demonstrated an increase in expression of LYVE-1 in draining lymph nodes compared to vehicle-treated mice, indicating an increase in endothelial cell activity and lymphangiogenesis.

### EQUIVALENTS

The present disclosure is not to be limited in scope by the specific embodiments described herein. Indeed, various modifications of the subject matter provided herein, in addition to those described, will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

What is claimed is:

1. A method of treating an individual having lymphedema, or a disease or disorder caused by lymphedema, comprising administering to the individual a therapeutically effective amount of placental adherent cells, wherein said therapeutically effective amount is an amount sufficient to reduce or ameliorate one or more symptoms or sequelae of said lymphedema or disease or disorder caused by lymphedema, in said individual, as compared to the individual prior to said administering.

2. The method of claim 1, wherein said one or more symptoms or sequelae of said lymphedema or disease or disorder caused by lymphedema is: swelling of part or all of one or both legs, swelling of part or all of one or both arms, heaviness or tightness in an arm or leg, nontender pitted edema of a leg or arm, restricted motion or a sensation of restricted motion in an arm or leg, aching or discomfort in an arm or leg, infection in or below the skin, cellulitis, lymphangitis, lymphorrhea, erythema of the skin of an affected arm or leg, a positive Stemmer's sign, and/or hardening and/or thickening of the skin of an affected arm or leg.

3. The method of claim 1 or 2, wherein said lymphedema is not caused by, or associated with, venous insufficiency, heart failure, sleep apnea, or kidney failure.

4. The method of claim 1, wherein said one or more symptoms or sequelae of said lymphedema or disease or disorder caused by lymphedema is infection.

5. The method of claim 4, wherein said infection is accompanied by impetigo.

6. The method of claim 1, wherein said one or more symptoms or sequelae of said lymphedema or disease or disorder caused by lymphedema is elephantiasis nostra verrucosa.

7. The method of any one of claims 1-6, wherein said lymphedema is lymphedema adjunct to lipedema.

8. The method of any one of claims 1-7, wherein said lymphedema is caused in part or whole by breast cancer surgery.

9. The method of any one of claims 1-8, wherein said lymphedema is Stage 0 lymphedema.

10. The method of any one of claims 1-8, wherein said lymphedema is Stage 1 lymphedema.

11. The method of any one of claims 1-8, wherein said lymphedema is Stage 2 lymphedema.

12. The method of any one of claims 1-8, wherein said lymphedema is Stage 3 lymphedema.

13. The method of any of claims 1-12, wherein said placental adherent cells are CD34⁻, CD10⁺, CD105⁺ and CD200⁺, as detectable by flow cytometry.

14. The method of claim 13, wherein said placental adherent cells have the capacity to differentiate into cells having one or more characteristics of osteogenic or chondrogenic cells.

15. The method of claim 13, wherein said placental adherent cells are additionally one or more of CD38, CD45, CD80, CD86, CD133, HLA-DR,DP,DQ, SSEA3, SSEA4⁻, CD29⁺, CD44⁺, CD73⁺, CD90⁺, CD105⁺, HLA-A,B,C⁺, PDL1⁺, ABC-p⁺, and/or OCT-4⁺, as detectable by flow cytometry and/or RT-PCR.

16. The method of any of claims 1-12, wherein said placental adherent cells are CD34⁻, CD45⁻, CD10⁺, CD90⁺, CD105⁺ and CD200⁺, as detectable by flow cytometry.

17. The method of any of claims 1-12, wherein said placental adherent cells are CD34⁻, CD45⁻, CD10⁺, CD80⁻, CD86⁻, CD90⁺, CD105⁺ and CD200, as detectable by flow cytometry.

18. The method of any of claims 1-12, wherein said placental adherent cells are CD34⁻, CD45⁻, CD10, CD80⁻, CD86⁻, CD90⁺, CD105⁺ and CD200, and additionally one or more of CD29⁺, CD38⁻, CD44⁺, CD54⁺, SH3⁺ or SH4⁺, as detectable by flow cytometry.

19. The method of any of claims 1-12, wherein said placental adherent cells are CD34, CD38, CD45, CD10⁺, CD29⁺, CD44⁺, CD54⁺, CD73⁺, CD80, CD86, CD90⁺, CD105⁺, and CD200⁺ as detectable by flow cytometry.

20. The method of claim 13, wherein said CD34, CD10⁺, CD105⁺ and CD200⁺ placental adherent cells are additionally one or more of CD117⁻, CD133⁻, KDR⁻ (VEGFR2⁻), HLA-A,B,C⁺, HLA-DP,DQ,DR⁻, or Programmed Death-1 Ligand (PDL1)⁺, or any combination thereof.

21. The method of any of claims 1-12, wherein said placental adherent cells are CD34⁻, CD38⁻, CD45⁻, CD10⁺, CD29⁺, CD44⁺, CD54⁺, CD73⁺, CD80⁻, CD86⁻, CD90⁺, CD105⁺, CD117⁻, CD133⁻, CD200⁺, KDR⁻ (VEGFR2⁻), HLA-A,B,C⁺, HLA-DP,DQ,DR⁻, or Programmed Death-1 Ligand (PDL1)⁺, as detectable by flow cytometry.

22. The method of any of claims 13-21, wherein said placental adherent cells are additionally ABC-p⁺, as detectable by flow cytometry, or OCT-4⁺ (POU5F1⁺) as determinable by RT-PCR.

23. The method of any of claims 13-21, wherein said placental adherent cells are additionally SSEA3⁻ or SSEA4⁻, as determinable by flow cytometry, wherein SSEA3 is Stage Specific Embryonic Antigen 3, and SSEA4 is Stage Specific Embryonic Antigen 4.

24. The method of any of claims 13-21, wherein said placental adherent cells, express one or more genes at a detectably higher level than an equivalent number of bone marrow-derived mesenchymal stem cells, wherein said one or more genes are one or more of ACTG2, ADARB1, AMIGO2, ARTS-1, B4GALT6, BCHE, C11orf9, CD200, COL4A1, COL4A2, CPA4, DMD, DSC3, DSG2, ELOVL2, F2RL1, FLJ10781, GATA6, GPR126, GPRC5B, ICAM1, IER3, IGFBP7, IL1A, IL6, IL18, KRT18, KRT8, LIPG, LRAP, MATN2, MEST, NFE2L3, NUAK1, PCDH7, PDLIM3, PKP2, RTN1, SERPINB9, ST3GAL6, ST6GALNAC5, SLC12A8, TCF21, TGFB2, VTN, and ZC3H12A, and wherein said bone marrow-derived mesenchymal stem cells have undergone a number of passages in culture equivalent to the number of passages said isolated placental adherent cells have undergone.
